SEQUENCE LISTING (28 sequences)

SEQ ID NO: 1 - DNA, 2670 bp, Homo sapiens
atggacgcgcgcgggggcggcgggcggcccggggagagcccgggcgcgagccccacgaccgggccgccgccgccgccgccgcccgcgcccccccaacagcagccgccgccgccgccgccgcccgcgccccccccgggccccgggcccgcgcccccccagcacccgccccgggccgaggcgttgcccccggaggcggcggatgagggcggcccgcggggccggctccgcagccgcgacagctcgtgcggccgccccggcaccccgggcgcggcgagcacggccaagggcagcccgaacggcgagtgcgggcgcggcgagccgcagtgcagccccgcggggcccgagggcccggcgcgggggcccaaggtgtcgttctcgtgccgcggggcggcctcggggcccgcgccggggccggggccggcggaggaggcgggcagcgaggaggcgggcccggcgggggagccgcgcggcagccaggccagcttcatgcagcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttttactgggatttaataatgcttataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtctgtggttgacttcatctcatccatcccagtggattatatctttcttattgtagaaaaaggaatggattctgaagtttacaagacagccagggcacttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggctgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaaggtgcagcatgacctcaactcgggcgtattcaacaaccaggagaacgccatcatccaggagatcgtcaagtacgaccgcgagatggtgcagcaggccgagctgggtcagcgcgtgggcctcttcccgccgccgccgccgccgccgcaggtcacctcggccatcgccacgctgcagcaggcggcggccatgagcttctgcccgcaggtggcgcggccgctcgtggggccgctggcgctcggctcgccgcgcctcgtgcgccgcccgcccccggggcccgcacctgccgccgcctcacccgggcccccgccccccgccagccccccgggcgcgcccgccagcccccgggcaccgcggacctcgccctacggcggcctgcccgccgccccccttgctgggcccgccctgcccgcgcgccgcctgagccgcgcgtcgcgcccactgtccgcctcgcagccctcgctgcctcacggcgcccccggccccgcggcctccacacgcccggccagcagctccacaccgcgcttggggcccacgcccgctgcccgggccgccgcgcccagcccggaccgcagggactcggcctcacccggcgccgccggcggcctggacccccaggactccgcgcgctcgcgcctctcgtccaacttgtga

SEQ ID NO: 2 - protein, 889 aa, Homo sapiens
MDARGGGGRPGESPGASPTTGPPPPPPPAPPQQQPPPPPPPAPPPGPGPAPPQHPPRAEALPPEAADEGGPRGRLRSRDSSCGRPGTPGAASTAKGSPNGECGRGEPQCSPAGPEGPARGPKVSFSCRGAASGPAPGPGPAEEAGSEEAGPAGEPRGSQASFMQRQFGALLQPGVNKFSLRMFGSQKAVEREQERVKSAGAWIIHPYSDFRFYWDLIMLIMMVGNLVIIPVGITFFTEQTTTPWIIFNVASDTVFLLDLIMNFRTGTVNEDSSEIILDPKVIKMNYLKSWSVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLNSGVFNNQENAIIQEIVKYDREMVQQAELGQRVGLFPPPPPPPQVTSAIATLQQAAAMSFCPQVARPLVGPLALGSPRLVRRPPPGPAPAAASPGPPPPASPPGAPASPRAPRTSPYGGLPAAPLAGPALPARRLSRASRPLSASQPSLPHGAPGPAASTRPASSSTPRLGPTPAARAAAPSPDRRDSASPGAAGGLDPQDSARSRLSSNL

SEQ ID NO: 3 - DNA, 2325 bp, Homo sapiens
atggaggcagagcagcggccggcggcgggggccagcgaaggggcgacccctggactggaggcggtgcctcccgttgctcccccgcctgcgaccgcggcctcaggtccgatccccaaatctgggcctgagcctaagaggaggcaccttgggacgctgctccagcctacggtcaacaagttctcccttcgggtgttcggcagccacaaagcagtggaaatcgagcaggagcgggtgaagtcagcgggggcctggatcatccacccctacagcgacttccggttttactgggacctgatcatgctgctgctgatggtggggaacctcatcgtcctgcctgtgggcatcaccttcttcaaggaggagaactccccgccttggatcgtcttcaacgtattgtctgatactttcttcctactggatctggtgctcaacttccgaacgggcatcgtggtggaggagggtgctgagatcctgctggcaccgcgggccatccgcacgcgctacctgcgcacctggttcctggttgacctcatctcttctatccctgtggattacatcttcctagtggtggagctggagccacggttggacgctgaggtctacaaaacggcacgggccctacgcatcgttcgcttcaccaagatcctaagcctgctgaggctgctccgcctctcccgcctcatccgctacatacaccagtgggaggagatctttcacatgacctatgacctggccagtgctgtggttcgcatcttcaacctcattgggatgatgctgctgctatgtcactgggatggctgtctgcagttcctggtgcccatgctgcaggacttccctcccgactgctgggtctccatcaaccacatggtgaaccactcgtggggccgccagtattcccatgccctgttcaaggccatgagccacatgctgtgcattggctatgggcagcaggcacctgtaggcatgcccgacgtctggctcaccatgctcagcatgatcgtaggtgccacatgctacgccatgttcatcggccatgccacggcactcatccagtccctggactcttcccggcgtcagtaccaggagaagtacaagcaggtggagcagtacatgtccttccacaagctgccagcagacacgcggcagcgcatccacgagtactatgagcaccgctaccagggcaagatgttcgatgaggaaagcatcctgggcgagctgagcgagccgcttcgcgaggagatcattaacttcacctgtcggggcctggtggcccacatgccgctgtttgcccatgccgaccccagcttcgtcactgcagttctcaccaagctgcgctttgaggtcttccagccgggggatctcgtggtgcgtgagggctccgtggggaggaagatgtacttcatccagcatgggctgctcagtgtgctggcccgcggcgcccgggacacacgcctcaccgatggatcctactttggggagatctgcctgctaactaggggccggcgcacagccagtgttcgggctgacacctactgccgcctttactcactcagcgtggaccatttcaatgctgtgcttgaggagttccccatgatgcgccgggcctttgagactgtggccatggatcggctgctccgcatcggcaagaagaattccatactgcagcggaagcgctccgagccaagtccaggcagcagtggtggcatcatggagcagcacttggtgcaacatgacagagacatggctcggggtgttcggggtcgggccccgagcacaggagctcagcttagtggaaagccagtactgtgggagccactggtacatgcgccccttcaggcagctgctgtgacctccaatgtggccattgccctgactcatcagcggggccctctgcccctctcccctgactctccagccaccctccttgctcgctctgcttggcgctcagcaggctctccagcttccccgctggtgcccgtccgagctggcccatgggcatccacctcccgcctgcccgccccacctgcccgaaccctgcacgccagcctatcccgggcagggcgctcccaggtctccctgctgggtccccctccaggaggaggtggacggcggctaggacctcggggccgcccactctcagcctcccaaccctctctgcctcagcgggcaacaggcgatggctctcctgggcgtaagggatcaggaagtgagcggctgcctccctcagggctcctggccaaacctccaaggacagcccagccccccaggccaccagtgcctgagccagccacaccccggggtctccagctttctgccaacatgtaa

SEQ ID NO: 4 - protein, 774 aa, Homo sapiens
MEAEQRPAAGASEGATPGLEAVPPVAPPPATAASGPIPKSGPEPKRRHLGTLLQPTVNKFSLRVFGSHKAVEIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPPDCWVSINHMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLLRIGKKNSILQRKRSEPSPGSSGGIMEQHLVQHDRDMARGVRGRAPSTGAQLSGKPVLWEPLVHAPLQAAAVTSNVAIALTHQRGPLPLSPDSPATLLARSAWRSAGSPASPLVPVRAGPWASTSRLPAPPARTLHASLSRAGRSQVSLLGPPPGGGGRRLGPRGRPLSASQPSLPQRATGDGSPGRKGSGSERLPPSGLLAKPPRTAQPPRPPVPEPATPRGLQLSANM

SEQ ID NO: 5 - DNA, 2592 bp, Mus musculus
atggatgcgcgcgggggcggcgggcggccgggcgatagtccgggcacgacccctgcgccggggccgccgccaccgccgccgccgcccgcgccccctcagcctcagccaccacccgcgccacccccgaaccccacgaccccctcgcacccggagtcggcggacgagcccggcccgcgcgcccggctctgcagccgcgacagcgcctgcacccctggcgcggccaagggcggcgcgaatggcgagtgcgggcgcggggagccgcagtgcagccccgagggccccgcgcgcggccccaaggtttcgttctcatgccgcggggcggcctccgggccctcggcggccgaggaggcgggcagcgaggaggcgggcccggcgggtgagccgcgcggcagccaggctagcttcctgcagcgccaattcggggcgcttctgcagcccggcgtcaacaagttctccctgcggatgttcggcagccagaaggccgtggagcgcgagcaggaacgcgtgaagtcggcgggggcctggatcatccacccctacagcgacttcaggttttattggggattaatcatgcttataatgatggttggaaatttggtcatcataccagttggaatcacgttcttcacagagcagacgacaacaccgtggattattttcaacgtggcatccgatactgttttcctgttggacttaatcatgaattttaggactgggactgtcaatgaagacagctcggaaatcatcctggaccctaaagtgatcaagatgaattatttaaaaagctggtttgtggtggacttcatctcatcgatcccggtggattatatctttctcattgtagagaaagggatggactcagaagtttacaagacagccagagcacttcgtatcgtgaggtttacaaaaattctcagtctcttgcggttattacgcctttcaaggttaatcagatacatacaccagtgggaagagatattccacatgacctatgacctcgccagtgctgtggtgaggatcttcaacctcattggcatgatgctgcttctgtgccactgggatggctgtcttcagttcctggttcccctgctgcaggacttcccaccagattgctgggtttctctgaatgaaatggttaatgattcctggggaaaacaatattcctacgcactcttcaaagctatgagtcacatgctgtgcattggttatggcgcccaagcccctgtcagcatgtctgacctctggattaccatgctgagcatgattgtgggcgccacctgctacgcaatgtttgttggccatgccacagctttgatccagtctttggattcgtcacggcgccaataccaggagaagtacaagcaagtagagcaatacatgtccttccacaaactgcccgctgacttccgccagaagatccacgattactatgaacaccggtaccaagggaagatgtttgatgaggacagcatccttggggaactcaacgggccactgcgtgaggagattgtgaacttcaactgccggaagctggtggcttccatgccgctgtttgccaatgcagaccccaacttcgtcacagccatgctgacaaagctcaaatttgaggtcttccagcctggagattacatcatccgagaggggaccatcgggaagaagatgtacttcatccagcatggggtggtgagcgtgctcaccaagggcaacaaggagatgaagctgtcggatggctcctatttcggggagatctgcttgctcacgaggggccggcgtacggccagcgtgcgagctgacacctactgtcgcctctactcactgagtgtggacaatttcaacgaagtactggaggaataccccatgatgcggcgtgcctttgagactgtggctattgaccggctagatcgcataggcaagaagaactccatcttgctgcacaaggttcagcatgatctcagctcaggtgtgttcaacaaccaggagaatgccatcatccaggagattgtcaaatatgaccgtgagatggtgcagcaggcagagcttggacagcgtgtggggctcttcccaccaccgccaccaccgcaggtcacatcggccattgccaccctacagcaggctgtggccatgagcttctgcccgcaggtggcccgcccgctcgtggggcccctggcgctaggctccccacgcctagtgcgccgcgcgcccccagggcctctgcctcctgcagcctcgccagggccacccgcagcaagccccccggctgcaccctcgagccctcgggcaccgcggacctcaccctacggtgtgcctggctctccggcaacgcgtgtggggcccgcattgcccgcacgtcgcctgagccgcgcctcgcgcccactgtccgcctcgcagccctcgctgccccatggcgtgcccgcgcccagccccgcggcctctgcgcgcccggccagcagctccacgccgcgcctgggacccgcacccaccgcccggaccgccgcgcccagtccggaccgcagggactcagcctcgccgggcgctgccagtggcctcgacccactggactctgcgcgctcgcgcctctcttccaacttgtga

SEQ ID NO: 6 - protein, 863 aa, Mus musculus
MDARGGGGRPGDSPGTTPAPGPPPPPPPPAPPQPQPPPAPPPNPTTPSHPESADEPGPRARLCSRDSACTPGAAKGGANGECGRGEPQCSPEGPARGPKVSFSCRGAASGPSAAEEAGSEEAGPAGEPRGSQASFLQRQFGALLQPGVNKFSLRMFGSQKAVEREQERVKSAGAWIIHPYSDFRFYWGLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLSSGVFNNQENAIIQEIVKYDREMVQQAELGQRVGLFPPPPPPQVTSAIATLQQAVAMSFCPQVARPLVGPLALGSPRLVRRAPPGPLPPAASPGPPAASPPAAPSSPRAPRTSPYGVPGSPATRVGPALPARRLSRASRPLSASQPSLPHGVPAPSPAASARPASSSTPRLGPAPTARTAAPSPDRRDSASPGAASGLDPLDSARSRLSSNL

SEQ ID NO: 7 - DNA, 2340 bp, Mus musculus
atggaggaggaggcgcggccggcggcgggggccggcgaagcggcgacccctgcacgcgagacgcctcctgcggctccggcccaggcccgcgcggcctcaggtggggtgccggagtctgcgcccgagccgaagaggcggcagctcgggacgctgctgcagccgacggtcaacaagttctctctccgggtcttcggcagccacaaagcagtagaaatcgagcaggagagggtgaagtccgccggggcctggatcatccacccctacagcgacttccggttttactgggatctcatcatgctgctgctgatggtggggaacctcatagttctgcctgtgggtatcactttcttcaaggaggagaactctccaccctggatcgtcttcaatgtcctctctgacactttcttcctgctggatctggtgctcaacttccgaactggcatcgtggtggaggaaggtgccgagatcctgctggcgccaagggccatccgaacgcgttacctgcgcacctggttcctggttgatctgatctcctccatccctgtggattatatcttcctagtggtggagctggagccacgactagatgctgaggtctacaaaacggcacgggccctgcgcatcgttagattcaccaagatccttagcctgctgcggctgctccgcctctcccgcctcatccgctacatacaccagtgggaggagatctttcacatgacctacgacctggccagtgcagtggttcgcatcttcaacctcattggaatgatgttgctgctgtgtcactgggacggctgtctgcagtttctggtccctatgctgcaggacttcccgtccgactgctgggtctccatgaaccgcatggtgaaccactcgtggggccgccagtattcccacgccctgttcaaggccatgagtcacatgctatgcattggctatgggcagcaggcaccggtaggcatgcctgacgtctggctcaccatgctcagtatgattgtgggcgccacgtgttatgccatgttcatcggtcacgccaccgccctcatccagtccctggactcttcccggcgacagtaccaggagaagtacaagcaggtggagcagtacatgtccttccacaagctgcccgctgacacccggcagcgcatccacgagtactacgagcatcgctaccagggcaagatgtttgatgaagagagcatcctgggggagctgagcgagccacttcgggaggagattattaacttcacctgccggggcctggtggcccacatgccgctgtttgctcatgctgaccccagcttcgtcaccgcagtgctcaccaagctccgttttgaggtcttccaaccaggggacctggtggtgcgtgagggctccgtgggcaggaagatgtacttcatccagcacgggctgctgagtgtgctggcacgtggcgcccgcgacacccgcctcactgatggatcctactttggggagatctgcctgctgactcgaggtcggagaacagccagtgtaagggctgacacctattgtcgcctctactcgctcagcgtggaccacttcaatgcggtgcttgaggagttcccaatgatgcgcagggcttttgagacggtggccatggaccggcttcggcgcatcggcaaaaagaattcgatactgcagcggaaacgctctgagccgagtccaggcagcagcggtggcgtcatggagcagcatttggtacaacacgacagagacatggctcgtggtgttcggggcctggctcctggtacaggagctcgactcagtggaaagccagtgctgtgggaaccactggtgcacgcccctctgcaggcagctgctgtgacctccaacgtggccatagccttgactcaccagcgaggccctctgcccctctcccctgattctccagccaccctcctagctcgatctgctagacgctcagcaggctccccagcctccccactggtgcctgtccgagcaggtcctctgctggcccggggaccctgggcgtccacttctcgcctgcctgctccacctgcccgaaccctccatgccagcctatcccggacagggcgttcccaggtatctctgttgggccctcccccaggaggaggtgctcggaggctaggacctcggggccgcccactttctgcctcgcaaccctctctgcctcagcgagcaacaggggatggctctcctaggcgtaaaggctctggaagtgagcgcctgcccccctctgggctcttggccaaacctccagggacagtccagccacccaggtcatcagtgcctgagccagttacccccagaggtccccaaatttctgccaacatgtga

SEQ ID NO: 8 - protein, 779 aa, Mus musculus
MEEEARPAAGAGEAATPARETPPAAPAQARAASGGVPESAPEPKRRQLGTLLQPTVNKFSLRVFGSHKAVEIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPSDCWVSMNRMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLRRIGKKNSILQRKRSEPSPGSSGGVMEQHLVQHDRDMARGVRGLAPGTGARLSGKPVLWEPLVHAPLQAAAVTSNVAIALTHQRGPLPLSPDSPATLLARSARRSAGSPASPLVPVRAGPLLARGPWASTSRLPAPPARTLHASLSRTGRSQVSLLGPPPGGGARRLGPRGRPLSASQPSLPQRATGDGSPRRKGSGSERLPPSGLLAKPPGTVQPPRSSVPEPVTPRGPQISANM

SEQ ID NO: 9 - protein, 910 aa, Mus musculus
MEGGGKPNSASNSRDDGNSVFPSKAPATGPVAADKRLGTPPRGGAAGKEHGNSVCFKVDGGGGEEPAGSFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILSELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIPPINYPQMTALNCTSSTTTPTSRMRTQSPPVYTATSLSHSNLHSPSPSTQTPQPSAILSPCSYTTAVCSPPIQSPLATRTFHYASPTASQLSLMQQPQQQLPQSQVQQTQTQTQQQQQQQQQQQQQQQQQQQQQQQQQQQQQQQQQQQQQPQTPGSSTPKNEVHKSTQALHNTNLTKEVRPLSASQPSLPHEVSTLISRPHPTVGESLASIPQPVAAVHSTGLQAGSRSTVPQRVTLFRQMSSGAIPPNRGVPPAPPPPAAVQRESPSVLNTDPDAEKPRFASNL

SEQ ID NO: 10 - protein, 910 aa, Rattus norvegicus
MEGGGKPNSASNSRDDGNSVYPSKAPATGPAAADKRLGTPPGGGAAGKEHGNSVCFKVDGGGGEEPAGSFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILSELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIPPINYPQMTALNCTSSTTTPTSRMRTQSPPVYTATSLSHSNLHSPSPSTQTPQPSAILSPCSYTTAVCSPPIQSPLATRTFHYASPTASQLSLMQQPQPQLQQSQVQQTQTQTQQQQQQQQPQPQPQQPQQQQQQQQQQQQQQQQQQQQQPQTPGSSTPKNEVHKSTQALHNTHLTREVRPLSASQPSLPHEVSTMISRPHPTVGESLASIPQPVATVHSTGLQAGSRSTVPQRVTLFRQMSSGAIPPNRGVPPAPPPPAAVQRESPSVLNKDPDAEKPRFASNL

SEQ ID NO: 11 - protein, 890 aa, Homo sapiens
MEGGGKPNSSSNSRDDGNSVFPAKASATGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWSVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLRGEIVNFNCRKLVATMPLFANADPNFVTAMLSKLRFEVFQPGDYIVREGAVGKKMYFIQHGVAGVITKSSKEMKLTDGSYFGEICLLTKGRRTASVRADTYCRLYSLSVDNFNEVPEEYPMMRRAFETVAIDRLDRIGKKNSILLQKFQKDLNTGVFNNQENEILKQIVKHDREMVQAIAPINYPQMTTLNSASSTTTPTSRMRTQSPPVYTATSLSHSNLHSPSPSTQTPQPSAILSPCSYTTAVCSPPVQSPLAARTFHYASPTASQLSLMQQQPQQQVQQSQPPQTQPQQPSPQPQTPGSSTPKNEVHKSTQALHNTNLTREVRPLSASQPSLPHEVPTLISRPHPTVGESLASIPQPVTAVPGTGLQAGGRSTVPQRVTLFRQMSSGAIPPNRGVPPAPPPPAAALPRESSSVLNTDPDAEKPRFASNL

SEQ ID NO: 12 - protein, 822 aa, Oryctolagus cuniculus
MATASSPPRRPRRARGLEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIAPISYPQMTALNSTSSTATPTSRMRTQSPPVYTATSLSHSNLHSPSPSTQTPQPSAILSPCSYTTAVCSPPVQSPLATRTFHYASPTASQLSLMPQQQQQPQAPQTQPQQPPQQPQTPGSATPKNEVHRSTQALPNTSLTREVRPLSASQPSLPHEVSTLISRPHPTVGESLASIPQPVAAVHSAGLQAAGRSTVPQRVTLFRQMSSGAIPPNRGVPPAPPPPAAPLQREASSVLNTDPEAEKPRFASNL

SEQ ID NO: 13 - protein, 202 aa, Cavia porcellus
IMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNKMVNVSWGQQYSYALFKA

SEQ ID NO: 14 - protein, 863 aa, Mus musculus
MDARGGGGRPGDSPGTTPAPGPPPPPPPPAPPQPQPPPAPPPNPTTPSHPESADEPGPRARLCSRDSACTPGAAKGGANGECGRGEPQCSPEGPARGPKVSFSCRGAASGPSAAEEAGSEEAGPAGEPRGSQASFLQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPSDCWVSINNMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLSSGVFNNQENAIIQEIVKYDREMVQQAELGQRVGLFPPPPPPQVTSAIATLQQAVAMSFCPQVARPLVGPLALGSPRLVRRAPPGPLPPAASPGPPAASPPAAPSSPRAPRTSPYGVPGSPATRVGPALPARRLSRASRPLSASQPSLPHGVPAPSPAASARPASSSTPRLGPAPTARTAAPSPDRRDSASPGAASGLDPLDSARSRLSSNL

SEQ ID NO: 15 - protein, 863 aa, Rattus norvegicus
MDARGGGGRPGDSPGATPAPGPPPPPPPPAPPQPQPPPAPPPNPTTPSHPESADEPGPRSRLCSRDSSCTPGAAKGGANGECGRGEPQCSPEGPARGPKVSFSCRGAASGPAAAEEAGSEEAGPAGEPRGSQASFLQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPSDCWVSINNMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLSSGVFNNQENAIIQEIVKYDREMVQQAELGQRVGLFPPPPPPQVTSAIATLQQAVAMSFCPQVARPLVGPLALGSPRLVRRAPPGPLPPAASPGPPAASPPAAPSSPRAPRTSPYGVPGSPATRVGPALPARRLSRASRPLSASQPSLPHGAPAPSPAASARPASSSTPRLGPAPTTRTAAPSPDRRDSASPGAASGLDPLDSARSRLSSNL

SEQ ID NO: 16 - protein, 889 aa, Homo sapiens
MDARGGGGRPGESPGATPAPGPPPPPPPAPPQQQPPPPPPPAPPPGPGPAPPQHPPRAEALPPEAADEGGPRGRLRSRDSSCGRPGTPGAASTAKGSPNGECGRGEPQCSPAGPEGPARGPKVSFSCRGAASGPAPGPGPAEEAGSEEAGPAGEPRGSQASFMQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPRNCWVSINGMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLNSGVFNNQENAIIQEIVKYDREMVQQAELGQRVGLFPPPPPPPQVTSAIATLQQAAAMSFCPQVARPLVGPLALGSPRLVRRPPPGPAPAAASPGPPPPASPPGAPASPRAPRTSPYGGLPAAPLAGPALPARRLSRASRPLSASQPSLPHGAPGPAASTRPASSSTPRLRPTPAARAAAPSPDRRDSASPGAAGGLDPQDSARSRLSSNL

SEQ ID NO: 17 - protein, 97 aa, Canis familiaris
AMSHMLCIGYGRQAPESMTDIWLTMLSMIVGATCYAMFIGHATALIQSLDSSRRQYQEKYKQVEQYMSFHKLPADFRQKIHDYYEHRYQGKMFDEES

SEQ ID NO: 18 - protein, 1186 aa, Mus musculus
MDKLPPSMRKRLYSLPQQVGAKAWIMDEEEDGEEEGAGGRQDPSRRSIRLRPLPSPSPSVAAGCSESRGAALGATESEGPGRSAGKSSTNGDCRRFRGSLASLGSRGGGSGGAGGGSSLGHLHDSAEERRLIAAEGDASPGEDRTPPGLATEPERPATAAQPAASPPPQQPPQPASASCEQPSADTAIKVEGGAAAIDHILPEAEVRLGQSGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFVVDFISSIPVEYIFLIVETRIDSEVYKTARAVRIVRFTKILSLLRLLRLSRLIRYIHQWEEIFHMTYDLASAVVRIVNLIGMMLLLCHWDGCLQFLVPMLQDFPHDCWVSINGMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETRLADGSYFGEICLLTRGRRTASVRADTYCRLYSLSVDNFNEVLEEYPMMRKKNSILLHKVQHDLNSGVFNYQENEIIQQIVRHDREMAHCAHRVQAAASATPTPTPVIWTPLIQAPLQAAAATTSVAIALTHHPRLPAAIFRPPPGPGLGNLGAGQTPRHPRRLQSLIPSALGSASPASSPSQVDTPSSSSFHIQQLAGFSAPPGLSPLLPSSSSSPPPGACGSPPAPTPSTSTAAAASTTGFGHFHKALGGSLSSSDSPLLTPLQPGARSPQAAQPPPPLPGARGGLGLLEHFLPPPPSSRSPSSSPGQLGQPPGELSLGLAAGPSSTPETPPRPERPSFMAGASGGASPVAFTPRGGLSPPGHSPGPPRTFPSAPPRASGSHGSLLLPPASSPPPPQVPQRRGTPPLTPGRLTQDLKLISASQPALPQDGAQTLRRASPHSSGESVAAFSLYPRAGGGSGSSGGLGPPGRPYGAIPGQHVTLPRKTSSGSLPPPLSLFGARAASSGGPPLTTAAPQREPGARSEPVRSKLPSNL

SEQ ID NO: 19 - protein, 1198 aa, Rattus norvegicus
MDKLPPSMRKRLYSLPQQVGAKAWIMDEEEDGEEEGAGGLQDPSRRSIRLRPLPSPSPSVAAGCSESRGAALGAADSEGPGRSAGKSSTNGDCRRFRGSLASLGSRGGGSGGAGGGSSLGHLHDSAEERRLIAAEGDASPGEDRTPPGLATEPERPGAAAQPAASPPPQQPPQPASASCEQPSADTAIKVEGGAAASDQILPEAEVRLGQSGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFVVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPHDCWVSINGMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVRHDREMAHCAHRVQAAASATPTPTPVIWTPLIQAPLQAAAATTSVAIALTHHPRLPAAIFRPPPGPGLGNLGAGQTPRHPRRLQSLIPSALGSASPASSPSQVDTPSSSSFHIQQLAGFSAPPGLSPLLPSSSSSPPPGACSSPPAPTPSTSTAATTTGFGHFHKALGGSLSSSDSPLLTPLQPGARSPQAAQPPPPLPGARGGLGLLEHFLPPPPSSRSPSSSPGQLGQPPGELSPGLAAGPPSTPETPPRPERPSFMAGASGGASPVAFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFSLYPRAGGGSGSSGGLGPPGRPYGAIPGQHVTLPRKTSSGSLPPPLSLFGARAASSGGPPLTAAPQREPGARSEPVRSKLPSNL

SEQ ID NO: 20 - protein, 1203 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 21 - protein, 1175 aa, Oryctolagus cuniculus
MDKLPPSMRKRLYSLPQQVGAKAWIMDEEEDAEEEGAGGRQDPRRRSIRLRPLPSPSPSPSAAAAAAGGAESRGAALGGAADGEGPARGAAKSSTNGDCRRFRGSLASLGSRGGGGGGGSTGGGSHGHLHDSAEERRLIAEGDASPGEDRTPPGLAAEPERPGAPAPPAASPPQVPSSCGEQRPADAAVKVEGGAAAGDQILPEAEARLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTDIILDPRRIKMKYLKSWFVVDFVSSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSLNNMVNNSWGKQYSYALFKAMSHMLCIGYGRQAPMGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLSSGVSNYQENAIVQRIVQHDREMAHCARRAQATTPVAPAIWTPLIQAPLQAAAATTSVAIALTHHPRLPAAIFRPPPGPTTLGSLGAGQTPRHLRRLQSLAPSAPSPASPASSPSQPDTPSSASLHVQPLPGCSTPAGLGSLLPTAGSPPAPTPPTTAGAAGFSHFHRALGGSLSSSDSPLLTPMQSAARSPQQPPPPPGAPAGLGLLEHFLPPPARSPTSSPGQLGQPPGELSPGLGSGPPGTPETPPRQPERLPFAAGASAGASPVAFSPRGGPSPPGHSPGTPRTFPSAPPRASGSHGSLLLPPASSPPPPPPPPAPQRRATPPLAPGRLSQDLKLISASQPALPQDGAQTLRRASPHSSSGESVAALPPFPRAPGRPPGAGPGQHVTLTLPRKASSGSLPPPLSLFGPRAAPAGGPRLTAAPQREPGAKSEPVRSKLPSNL

SEQ ID NO: 22 - protein, 124 aa, Canis familiaris
DEDSILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDN

SEQ ID NO: 23 - DNA, 1528 bp, Homo sapiens
gaggcagttcacctccatgctgcagcccggggtcaacaaattctccctccgcatgtttgggagccagaaggcggtggaaaaggagcaggaaagggttaaaactgcaggcttctggattatccacccttacagtgatttcaggttttactgggatttaataatgcttataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtctgtggttgacttcatctcatccatcccagtggattatatctttcttattgtagaaaaaggaatggattctgaagtttacaagacagccagggcacttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggattcttcgaggcggcagtatcaagagaagtataagcaagtggaacaatacatgtcattccataagttaccagctgatatgcgtcagaagatacatgattactatgaacacagataccaaggcaaaatctttgatgaggaaaatattctcaatgaactcaatgatcctctgagaggggagatagtcaacttcaactgtcggaaactggtggctacaatgcctttatttgctaatgcggatcctaattttgtgactgccatgctgagcaagttgagatttgaggtgtttcaacctggagattatatcgtacgagaaggagccgtgggtaaaaaaatgtatttcattcaacacggtgttgctggtgtcattacaaaatccagtaaagaaatgaagctgacagatggctcttactttggagagatttgcctgctgaccaaaggacgtcgtactgccagtgttcgagctgatacatattgtcgtctttactcactttccgtggacaatttcaacgaggtcccggaggaatatccaatgatgaggagagcctttgagacagttgccattgaccgactagatcgaataggaaagaaaaattcaattcttctgcaaaag

SEQ ID NO: 24 - DNA, 1528 bp, Homo sapiens
gcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttctactgggacttcaccatgctgctgttcatggtgggaaacctcatcatcatcccagtgggcatcaccttcttcaaggatgagaccactgccccgtggatcgtgttcaacgtggtctcggacaccttcttcctcatggacctggtgttgaacttccgcaccggcattgtgatcgaggacaacacggagatcatcctggaccccgagaagatcaagaagaagtatctgcgcacgtggttcgtggtggacttcgtgtcctccatccccgtggactacatcttccttattgtggagaagggcattgactccgaggtctacaagacggcacgcgccctgcgcatcgtgcgcttcaccaagatcctcagcctcctgcggctgctgcgcctctcacgcctgatccgctacatccatcagtgggaggagatcttccacatgacctatgacctggccagcgcggtgatgaggatctgcaatctcatcagcatgatgctgctgctctgccactgggacggctgcctgcagttcctggtgcctatgctgcaggacttcccgcgcaactgctgggtgtccatcaatggcatggtgaaccactcgtggagtgaactgtactccttcgcactcttcaaggccatgagccacatgctgtgcatcgggtacggccggcaggcgcccgagagcatgacggacatctggctgaccatgctcagcatgattgtgggtgccacctgctacgccatgttcatcggccacgccactgccctcatccagtcgctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggccgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaag

SEQ ID NO: 25 - DNA, 1520 bp, Homo sapiens
gcgccagttcggggccatgctccaacccggggtcaacaaattctccctaaggatgttcggcagccagaaagccgtggagcgcgaacaggagagggtcaagtcggccggattttggattatccacccctacagtgacttcagattttactgggacctgaccatgctgctgctgatggtgggaaacctgattatcattcctgtgggcatcaccttcttcaaggatgagaacaccacaccctggattgtcttcaatgtggtgtcagacacattcttcctcatcgacttggtcctcaacttccgcacagggatcgtggtggaggacaacacagagatcatcctggacccgcagcggattaaaatgaagtacctgaaaagctggttcatggtagatttcatttcctccatccccgtggactacatcttcctcattgtggagacacgcatcgactcggaggtctacaagactgcccgggccctgcgcattgtccgcttcacgaagatcctcagcctcttacgcctgttacgcctctcccgcctcattcgatatattcaccagtgggaagagatcttccacatgacctacgacctggccagcgccgtggtgcgcatcgtgaacctcatcggcatgatgctcctgctctgccactgggacggctgcctgcagttcctggtacccatgctacaggacttccctgacgactgctgggtgtccatcaacaacatggtgaacaactcctgggggaagcagtactcctacgcgctcttcaaggccatgagccacatgctgtgcatcggctacgggcggcaggcgcccgtgggcatgtccgacgtctggctcaccatgctcagcatgatcgtgggtgccacctgctacgccatgttcattggccacgccactgccctcatccagtccctggactcctcccggcgccagtaccaggaaaagtacaagcaggtggagcagtacatgtcctttcacaagctcccgcccgacacccggcagcgcatccacgactactacgagcaccgctaccagggcaagatgttcgacgaggagagcatcctgggcgagctaagcgagcccctgcgggaggagatcatcaactttaactgtcggaagctggtggcctccatgccactgtttgccaatgcggaccccaacttcgtgacgtccatgctgaccaagctgcgtttcgaggtcttccagcctggggactacatcatccgggaaggcaccattggcaagaagatgtacttcatccagcatggcgtggtcagcgtgctcaccaagggcaacaaggagaccaagctggccgacggctcctactttggagagatctgcctgctgacccggggccggcgcacagccagcgtgagggccgacacctactgccgcctctactcgctgagcgtggacaacttcaatgaggtgctggaggagtaccccatgatgcgaagggccttcgagaccgtggcgctggaccgcctggaccgcattggcaagaagaactccatcctcc

SEQ ID NO: 26 - DNA, 1527 bp, Mus musculus
gaggcagttcacctccatgctgcagcctggggtcaacaaattctccctccgcatgtttgggagccagaaggcggtggagaaggagcaggaaagggttaaaactgcaggcttctggattatccatccgtacagtgacttcaggttttattgggatttaatcatgcttataatgatggttggaaatttggtcatcataccagttggaatcacgttcttcacagagcagacgacaacaccgtggattattttcaacgtggcatccgatactgttttcctgttggacttaatcatgaattttaggactgggactgtcaatgaagacagctcggaaatcatcctggaccctaaagtgatcaagatgaattatttaaaaagctggtttgtggtggacttcatctcatcgatcccggtggattatatctttctcattgtagagaaagggatggactcagaagtttacaagacagccagagcacttcgtatcgtgaggtttacaaaaattctcagtctcttgcggttattacgcctttcaaggttaatcagatacatacaccagtgggaagagatattccacatgacctatgacctcgccagtgctgtggtgaggatcttcaacctcattggcatgatgctgcttctgtgccactgggatggctgtcttcagttcctggttcccctgctgcaggacttcccaccagattgctgggtttctctgaatgaaatggttaatgattcctggggaaaacaatattcctacgcactcttcaaagctatgagtcacatgctgtgcattggttatggcgcccaagcccctgtcagcatgtctgacctctggattaccatgctgagcatgattgtgggcgccacctgctacgcaatgtttgttggccatgccacagctttgatccagtctttggactcttcaaggaggcagtatcaagagaagtataagcaagtagagcaatacatgtcattccacaagttaccagctgacatgcgccagaagatacatgattactatgagcaccgataccaaggcaagatcttcgatgaagaaaatattctcagtgagcttaatgatcctctgagagaggaaatagtcaacttcaactgccggaaactggtggctactatgcctctttttgctaacgccgatcccaatttcgtgacggccatgctgagcaagctgagatttgaggtgttccagcccggagactatatcattcgagaaggagctgtggggaagaaaatgtatttcatccagcacggtgttgctggcgttatcaccaagtccagtaaagaaatgaagctgacagatggctcttacttcggagagatatgcctgctgaccaagggccggcgcactgccagtgtccgagctgatacctactgtcgtctttactccctttcggtggacaatttcaatgaggtcttggaggaatatccaatgatgagaagagcctttgagacagttgctattgaccgactcgatcggataggcaagaaaaactctattctcctgcagaa

SEQ ID NO: 27 - DNA, 1527 bp, Mus musculus
gcgccaattcggggcgcttctgcagcccggcgtcaacaagttctccctgcggatgttcggcagccagaaggccgtggagcgcgagcaggaacgcgtgaagtcggcgggggcctggatcatccacccctacagcgacttcaggttctactgggacttcaccatgctgttgttcatggtgggaaatctcattatcattcccgtgggcatcactttcttcaaggacgagaccaccgcgccctggatcgtcttcaacgtggtctcggacactttcttcctcatggacttggtgttgaacttccgcaccggcattgttattgaggacaacacggagatcatcctggaccccgagaagataaagaagaagtacttgcgtacgtggttcgtggtggacttcgtgtcatccatcccggtggactacatcttcctcatagtggagaagggaatcgactccgaggtctacaagacagcgcgtgctctgcgcatcgtgcgcttcaccaagatcctcagtctgctgcggctgctgcggctatcacggctcatccgatatatccaccagtgggaagagattttccacatgacctacgacctggcaagtgcagtgatgcgcatctgtaacctgatcagcatgatgctactgctctgccactgggacggttgcctgcagttcctggtgcccatgctgcaagacttccccagcgactgctgggtgtccatcaacaacatggtgaaccactcgtggagcgagctctactcgttcgcgctcttcaaggccatgagccacatgctgtgcatcggctacgggcggcaggcgcccgagagcatgacagacatctggctgaccatgctcagcatgatcgtaggcgccacctgctatgccatgttcattgggcacgccactgcgctcatccagtccctggattcgtcacggcgccaataccaggagaagtacaagcaagtagagcaatacatgtccttccacaaactgcccgctgacttccgccagaagatccacgattactatgaacaccggtaccaagggaagatgtttgatgaggacagcatccttggggaactcaacgggccactgcgtgaggagattgtgaacttcaactgccggaagctggtggcttccatgccgctgtttgccaatgcagaccccaacttcgtcacagccatgctgacaaagctcaaatttgaggtcttccagcctggagattacatcatccgagaggggaccatcgggaagaagatgtacttcatccagcatggggtggtgagcgtgctcaccaagggcaacaaggagatgaagctgtcggatggctcctatttcggggagatctgcttgctcacgaggggccggcgtacggccagcgtgcgagctgacacctactgtcgcctctactcactgagtgtggacaatttcaacgaggtgctggaggaataccccatgatgcggcgtgcctttgagactgtggctattgaccggctagatcgcataggcaagaagaactccatcttgctgcacaa

SEQ ID NO: 28 - DNA, 1547 bp, Mus musculus
gcgcctgggccagagcggcttcatgcagcgccagttcggtgccatgctgcaacctggggtcaacaaattctccctaaggatgttcggcagccagaaagcggtggagcgcgagcaggagagggttaagtcagcagggttttggattatccacccctacagtgacttcagattttactgggacctgacgatgctgttgctgatggtggggaatctgatcatcatacccgtgggcatcaccttcttcaaggatgagaacaccacaccctggatcgtcttcaatgtggtgtcagacacattcttcctcattgacttggtcctcaacttccgcacggggatcgtggtggaggacaacacagaaatcatccttgacccgcagaggatcaagatgaagtacctgaaaagctggtttgtggtagatttcatctcctccatccctgtcgactacatcttccttatagtggagactcgcattgactcggaggtctacaaaaccgctagggctctgcgcattgtccgtttcactaagatcctcagcctcctgcgcctcttgaggctttcccgcctcattcgatacattcatcagtgggaagagatcttccacatgacctatgacctggccagcgccgtggtacgcatcgtgaacctcattggcatgatgcttctgctgtgtcactgggatggctgcctgcagttcctagtgcccatgctgcaggacttcccccatgactgctgggtgtccatcaatggcatggtgaataactcctgggggaagcagtattcctacgccctcttcaaggccatgagccacatgctgtgcattgggtatggacggcaggcacccgtaggcatgtctgacgtctggctcaccatgctcagcatgatcgtgggggccacctgctatgccatgttcatcggccacgccactgccctcatccagtcgctagactcctcccggcgccagtaccaggagaagtataaacaggtggagcagtacatgtccttccacaagctcccgcctgacacccgacagcgcatccatgactactatgaacaccgctaccaaggcaagatgtttgatgaggaaagcatcctgggtgagctgagtgagccacttcgagaggagatcatcaactttaactgccgaaagctggtggcatccatgccactgtttgccaacgcagatcccaactttgtgacatccatgctgaccaagttgcgtttcgaggtcttccagcctggggattacatcatccgcgaaggcaccatcggcaagaagatgtactttatccagcacggcgtggtcagcgtgctcactaagggcaacaaagagaccaagctggctgatggctcctattttggagagatctgcttgctgacccggggtcggcgcacagccagcgtcagagcggatacttattgccgcctctactcactgagcgtggacaacttcaatgaggtgctggaggagtatcccatgatgcggagggccttcgagacggttgcgctggaccgcctggaccgcataggcaagaagaactccatcctcc

(19) United States
(12) Patent Application Publication
(10) Pub. No.: US 2009/0053180 A1
(43) Pub. Date: Feb. 26, 2009

(54) Tandem cardiac pacemaker system

(76) Inventors: Michael R. Rosen, New York, NY (US); Peter R. Brink, Setauket, NY (US); Richard B. Robinson, Cresskill, NJ (US); Ira S. Cohen, Stony Brook, NY (US); Steven Girouard, Chagrin Falls, OH (US); Bruce Kenknight, Maple Grove, MN (US)

Correspondence Address: KENYON & KENYON LLP, ONE BROADWAY, NEW YORK, NY, 10004, US

(21) Appl. No.: 11/490,997

(22) Filed: Jul. 21, 2006

Related U.S. Application Data
(60) Provisional application No. 60/701,312, filed on Jul. 21, 2005.
(60) Provisional application No. 60/715,934, filed on Sep. 9, 2005.
(60) Provisional application No. 60/781,723, filed on Mar. 14, 2006.

(51) Int. Cl. A61K 35/12; A61N 1/362; A61P 9/00

(52) U.S. Cl. 424/93.7; 607/3

(57) ABSTRACT

This invention provides pacemaker systems comprising (1) an electronic pacemaker, and (2) a biological pacemaker, wherein the biological pacemaker comprises a cell that functionally expresses a chimeric hyperpolarization-activated, cyclic nucleotide-gated (HCN) ion channel at a level effective to induce pacemaker current in the cell. The invention also provides related biological pacemakers, atrioventricular bridges, methods of making same, and methods of treating a subject afflicted with a cardiac rhythm disorder.

This application claims the benefit of U.S. Provisional Application Nos. 60/701,312, filed Jul. 21, 2005; 60/781,723, filed Mar. 14, 2006; and 60/715,934, filed Sep. 9, 2005, the contents of which are incorporated herein by reference in their entirety.

The invention disclosed herein was made with United States Government support under NIH Grant Nos. HL-28958, HL-20558, and HL-67101 from the National Institutes of Health. Accordingly, the United States Government has certain rights in this invention.

Throughout this application, various publications are referenced in parentheses by author name and date, patent number, or patent publication number. Full citations for these publications may be found at the end of the specification immediately preceding the claims. The disclosures of these publications in their entireties are hereby incorporated by reference into this application. However, the citation of a reference herein should not be construed as an acknowledgement that such reference is prior art to the present invention.

### FIELD OF THE INVENTION

The present invention relates to the generation and use of tandem cardiac pacemaker system comprising biological pacemakers based on expression of HCN channels and mutants and chimeras thereof, and their use in tandem with electronic pacemakers.

### BACKGROUND OF THE INVENTION

The mammalian heart generates a rhythm that is myogenic in origin. All the channels and transporters that are necessary to generate the rhythm of the heart reside in the myocytes. Regional variations in the abundance or characteristics of these elements are such that the rhythm originates in a specific anatomic location, the sinoatrial node. The sinoatrial node consists of only a few thousand electrically active pacemaker cells that generate spontaneous rhythmic action potentials that subsequently propagate to induce coordinated muscle contractions of the atria and ventricles. The rhythm is modulated, but not initiated, by the autonomic nervous system.

Malfunction or loss of pacemaker cells can occur due to disease or aging. For example, acute myocardial infarction kills millions of people each year and generally induces in survivors marked reductions in myocyte number and cardiac pump function. Adult cardiac myocytes divide only rarely, and the usual responses to myocyte cell loss include compensatory hypertrophy and/or congestive heart failure, a disease with a significant annual mortality.

Electronic pacemakers are lifesaving devices that provide a regular heartbeat in settings where the sinoatrial node, atrioventricular conduction, or both, have failed. They also have been adapted to the therapy of congestive heart failure. One of the major indications for electronic pacemaker therapy is high degree heart block, such that a normally functioning sinus node impulse cannot propagate to the ventricle. The result is ventricular arrest and/or fibrillation, and death. Another major indication for electronic pacemaker therapy is sinoatrial node dysfunction, in which the sinus node fails to initiate a normal heartbeat, thereby compromising cardiac output.

Despite their utility in treating heart block and/or sinoatrial node dysfunction, electronic pacemakers have certain disadvantages (Rosen et al., 2004; Rosen, 2005; Cohen et al., 2005). For example, they require regular monitoring and maintenance, including periodic pulse generator changes and the replacement of batteries and leads; they do not readily respond to the demands of exercise or emotion (although software has been developed to facilitate variations in heart rate while exercising); recent evidence suggests that long-term pacing could increase the risk of heart failure (Freudenberger et al., 2005); power pack and lead selection need to be adapted to the demands of growth and development in pediatric patients; there are limitations in sites where leads can be stably implanted which may compromise cardiac output to variable extents; problems can occur with infection which, while infrequent, can be catastrophic; they are expensive; and there is the potential for interference from other devices. So, although electronic pacemakers represent superb medical palliation, they are not a cure (Rosen et al., 2004). There is therefore a need for the development of alternatives that more completely reproduce normal function, e.g., by exhibiting autonomic responsiveness, and ultimately provide a cure (Rosen et al., 2004).

### SUMMARY OF THE INVENTION

The present invention provides a tandem pacemaker system comprising (1) an electronic pacemaker, and (2) a biological pacemaker, wherein the biological pacemaker comprises an implantable cell that functionally expresses a hyperpolarization-activated, cyclic nucleotide-gated (HCN) ion channel, and wherein the expressed HCN channel generates an effective pacemaker current when the cell is implanted into a subjects heart. The cell is preferably capable of gap junction mediated communication with cardiomyocytes, and is selected from the group consisting of a stem cell, a cardiomyocyte, a fibroblast or skeletal muscle cell engineered to express cardiac connexins, and an endothelial cell. In preferred embodiments, the stem cell is an embryonic or adult stem cell and wherein said stem cell is substantially incapable of differentiation.

In certain embodiments, the biological pacemaker comprises at least about 200,000 human adult mesenchymal stem cells, and preferably comprises at least about 700,000 human adult mesenchymal stem cells.

The HCN channel is HCN1, HCN2, HCN3 or HCN4, preferably human, or is an HCN channel has at least about 75% sequence identity with mHCN1, mHCN2, mHCN3, or mHCN4. The implantable cell may further functionally expresses a MiRP1 beta subunit.

The HCN may be a mutant HCN. Preferably the mutant HCN provides an improved characteristic, as compared to a wild-type HCN channel, selected from the group consisting of faster kinetics, more positive activation, increased expression, increased stability, enhanced cAMP responsiveness, and enhanced neurohumoral response.

The present invention also provides a tandem pacemaker system comprising (1) an electronic pacemaker, and (2) a bypass bridge comprising a strip of gap junction-coupled cells having a first end and a second end, both ends capable of being attached to two selected sites in a heart, so as to allow the transmission of an electrical signal across the tract between the two sites in the heart. Preferably the bypass bridge is an atrioventricular bypass bridge. In preferred embodiments, the cells are as described with respect to implantable cells of biological pacemakers of the present invention.

In certain embodiments, the cells of the bypass bridge functionally express at least one protein selected from the group consisting of: a cardiac connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel.

The present invention further provides a tandem pacemaker system comprising (1) an electronic pacemaker, and (2) a vector comprising a nucleic acid encoding an HCN channel or a mutant HCN channel, wherein said vector is administered to a cell in the heart of a subject and wherein said HCN channel or mutant HCN channel is expressed in the cells in the heart to generate an effective pacemaker current.

The present invention also provides methods of treating a subject afflicted with a cardiac rhythm disorder, which method comprises administering tandem pacemaker systems of the present invention.

The present invention also provides a tandem pacemaker system for treating a subject afflicted with ventricular dyssynchrony comprising (1) a biological pacemaker of the present invention to be administered to a site in one ventricle of the subject's heart, and (2) an electronic pacemaker to be administered to a site in the other ventricle of the subject's heart, wherein the electronic pacemaker is programmable to detect a signal from the biological pacemaker and to produce a electronic pacemaker signal at a reference time interval after the biological pacemaker signal is detected, so as to thereby provide biventricular pacemaker function, and wherein the electronic pacemaker is provided either prior or simultaneously with the biological pacemaker

### BRIEF DESCRIPTION OF THE FIGURES

FIG. 1. Initiation of spontaneous rhythms by wild-type or genetically engineered pacemaker cells as well as by genetically engineered stem cell pacemakers. Top, In a native pacemaker cell or in a myocyte engineered to incorporate pacemaker current via gene transfer, action potentials (inset) are initiated via inward current flowing through transmembrane HCN channels. These open when the membrane repolarizes to its maximum diastolic potential and close when the membrane has depolarized during the action potential. Current flowing via gap junctions to adjacent myocytes results in their excitation and the propagation of impulses through the conducting system. Bottom, A stem cell has been engineered to incorporate HCN channels in its membrane. These channels can only open, and current can only flow through them (inset) when the membrane is hyperpolarized; such hyperpolarization can only be delivered if an adjacent myocyte is tightly coupled to the stem cell via gap junctions. In the presence of such coupling and the opening of the HCN channels to induce local current flow, the adjacent myocyte will be excited and initiate an action potential that then propagates through the conducting system. The depolarization of the action potential will result in the closing of the HCN channels until the next repolarization restores a high negative membrane potential. Thus, wild-type and genetically engineered pacemaker cells incorporate in each cell all the machinery needed to initiate and propagate action potentials. In contrast, in the stem cell-myocyte pairing, two cells together work as a single functional unit whose operation is critically dependent on the gap junctions that form between the two disparate cell types.

FIG. 2. The role of I_{f} in generation of pacemaker potentials in the sinoatrial node (SAN). A, Pacemaker potentials in the SAN under control conditions, and after β-adrenergic stimulation with norepinephrine (NE). The four major currents that control the generation of the pacemaker potential are indicated: I_{f} current (produced by hyperpolarization-activated cyclic nucleotide-gated [HCN] channels), T-type (I_{CaT}) and L-type (I_{CaL}) calcium currents, and repolarizing K currents (I_{K}) B, Scheme of an SAN cell showing the regulation of the HCN channel by up- or downregulation of cellular cyclic adenosine monophosphate (cAMP). M2, type-2 muscarinic receptor; ACh, acetylcholine; AC, adenylyl cyclase; Gαi, G-protein α subunit (inhibits AC); Gβγ, G-protein βγ subunit; β1-AR, type-1 β-adrenergic receptor; Gas, G-protein α subunit (stimulates AC); ΔV, shift of the voltage dependence of HCN channel activation induced by increase or decrease of cAMP. (See Biel et al., 2002)

FIG. 3. Schematic representation of possible chimeric HCN channels. Illustrated are examples of channels constructed from elements of HCN2 (shown in light lines) and HCN1 (shown in dark lines), and designed to combine the rapid activation kinetics of HCN1 with the strong cAMP response of HCN2. The approach derives from the fact that the C-terminal cytoplasmic domain of the HCN channel contains the cyclic nucleotide binding domain and contributes significantly to cAMP responsiveness, whereas the transmembrane domain contributes significantly to the gating characteristics such as activation kinetics. Shown from top to bottom are: HCN2, HCN212 (in which the middle, transmembrane portion of HCN2 is replaced by the corresponding portion of HCN1), HCN112 (in which the C-terminal cytoplasmic portion of HCN1 is replaced by the corresponding portion of HCN2), and HCN1.

FIG. 4. Functional expression of mHCN2 and mE324A in newborn ventricular myocytes. Representative whole-cell current traces of ventricular myocytes infected with AdmHCN2 (A) or AdmE324A (B). Currents were evoked by stepping from a holding potential of −10 mV to different hyperpolarizing voltage steps ranging from −25 to −125 mV with increments of −10 mV. Insets at right shown the current traces recorded at −35, −45 and −55 mV at an expanded scale for both mHCN2 and mE324A. C, For illustrative purposes the mean activation data of mHCN2 (squares) and mE324A (circles) currents were fitted to the Boltzmann equation (lines). D, Voltage-dependence of activation (filled symbols) and deactivation (unfilled symbols) time constants of mHCN2 (squares) and mE324A (circles). Mean activation values were obtained from 14 cells for both mHCN2 and mE324A; mean deactivation time constants values were obtained from 8 and 7 cells for mHCN2 and mE324A respectively.

FIG. 5. Modulation of mHCN2 and mE324A by cAMP. Mean fractional activation curves of mHCN2 (squares) and mE324A (circles) obtained in the absence (unfilled symbols) and in the presence (filled symbols) of 10 μM cAMP in the pipette solution. The average data were fit to the Boltzmann equation for experiments in the absence (solid lines) and in the presence (dashed lines) of cAMP. Calculated values for mHCN2 were V_{1/2}=−69.6 mV and −59.9 (9.7 mV shift) and s=10.8 and 11.0 mV in the absence and in the presence of cAMP respectively. Calculated values for mE324A were V_{1/2}=−46.3 mV and −40.7 mV (5.6 mV) and s=9.1 mV and 8.7 mV in the absence and in the presence of cAMP respectively.

FIG. 6. Activation of expressed wild-type mHCN2 or mutant mE324A in oocytes. A and B, Activation of the expressed mHCN2 (A) or mE324A (B). Upper panels: Typical recordings of the activation of expressed mHCN2 and mE324A. The inset shows the pulse protocol used. For mHCN2, currents were elicited by 2-s long hyperpolarizing pulses between −30 mV and −160 mV with 10 mV increments, followed by a 1-s depolarizing pulse to +15 mV. The holding potential was −30 mV. For mE324A, currents were elicited by 3-s long hyperpolarizing pulses between +20 mV and −130 mV with 10 mV increments, followed by a 1-s depolarizing pulse to +50 mV. The holding potential was +20 mV. Middle panels: The corresponding tail currents used for the construction of steady state activation curves. Lower panels: The activation curves for mHCN2 or mE324A. The data were fit to the Boltzmann equation (1/[1+exp((V_{1/2}−Vₜₑₛₜ)/s)])). The half maximal activation (Vₕ) for mHCN2 was −92.7 mV±1.1 mV (n=9 cells), and currents saturated around −130 mV. A more positive activation threshold was noticed for mE324A (around −30 mV) and the Vₕ was −57.3 mV±1.6 mV (n=9 cells). C and D, Activation time constants of mHCN2 and mE324A. Note both a positive shift in voltage dependence and faster activation kinetics for mE324A.

FIG. 7. cAMP modulation of I_{HCN2} in oocytes injected with mHCN2 or mE324A. The Boltzmann fit of normalized ionic conductance showed that extracellular application of 8-Br-cAMP (cAMP, 1 mM) positively shifted the potential of half-maximal activation (Vₕ) of I_{HCN2} for both mHCN2 (left panel) and mE324A (right panel) by 7-8 mV.

FIG. 8. The pharmacological evaluation and the reversal potential of I_{HCN2} or mHCN2 and mE324A. A and B, The current/voltage relationships of I_{HCN2} for mHCN2 (A) and mE324A (B). Upper panels: The voltage protocols for the recording of the current/voltage relationship of I_{f}. For mHCN2, the cell was held at −30 mV, current was elicited by a 2-s hyperpolarizing voltage step to −140 mV to saturate activation, and followed by 2-s depolarizing voltage steps between −80 mV and +50 mV in 10 mV increments. For mE324A, the cell was held at +20 mV, current was elicited by a 1.5-s hyperpolarizing voltage step to −110 mV to saturate activation, and then followed by 1.5-s depolarizing voltage steps between −80 mV and +50 mV in 10 mV increments for the recording of tail currents. Lower panels: The representative traces used to construct the fully activated current/voltage relationship of I_{HCN2} in the presence of control, Cs⁺ (5 mM) and washout conditions, respectively. Note a large inhibition of the I_{f} by Cs⁺ for both mHCN2 and mE324A. C and D, The fully activated current/voltage curves of for mHCN2 (C) and mE324A (D) in the presence of control, Cs⁺ and washout conditions. The fully activated current/voltage relations were constructed by dividing the tail current magnitudes by the change in gating variable which occurred between the two test voltages (obtained from FIGS. 14A and B). The calculated reversal potential of I_{HCN2} is −41 mV for mHCN2 and −40 mV for mE324A.

FIG. 9. Comparison of current magnitude of I_{HCN2} in oocytes injected with mHCN2 or mE324A. The I_{HCN2} was measured at −120 mV for mHCN2 (n=10 cells) and mE324A (n=10 cells). Note the smaller current magnitude for the expressed mE324A (t-test, P<0.01). Voltage protocols are shown in the insets. For mHCN2, the current was evoked by applying a 3-s hyperpolarizing voltage pulse to −120 mV from a holding potential of −30 mV. For mE324A, the current was evoked by applying a 3-s hyperpolarizing voltage pulse to −120 mV from a holding potential of +20 mV.

FIG. 10. Identification of connexins in gap junctions of human mesenchymal stem cells (hMSCs). Immunostaining of Cx43 (A), Cx40 (B) and Cx45 (C). D, Immunoblot analysis of Cx43 in canine ventricle myocytes and hMSCs. Whole cell lysates (120 μg) from ventricle cells or hMSCs were resolved by SDS, transferred to membranes, and blotted with Cx43 antibodies. Molecular weight markers are indicated.

FIG. 11. Macroscopic and single channel properties of gap junctions between hMSC pairs. Gap junction currents (Iⱼ) elicited from hMSCs using a symmetrical bipolar pulse protocol (10 s, from ±10 mV to ±110 mV, Vₕ=0 mV) showed two types of voltage-dependent current deactivation: symmetrical (A) and asymmetrical (B). C, summary plots of normalized instantaneous (∘) and steady-state (•) gⱼ versus Vⱼ. Left panel, quasi-symetrical relationship from 5 pairs; continuous line, Boltzmann fit: V_{j,0}=−70/65 mV, g_{j,min}=0.29/0.34, g_{j,max}=0.99/1.00, z=2.2/2.3 for negative/positive Vⱼ. Right panel, asymmetrical relationship from 6 pairs; Boltzmann fit for negative Vⱼ: V_{j,0}=−72 mV, g_{j,min}=0.25, g_{j,max}=0.99, z=1.5. D and E, single channel recordings from pairs of hMSCs. Pulse protocol (V₁ and V₂) and associated multichannel currents (I₂) recorded from a cell pair during maintained Vⱼ of ±80 mV. The discrete current steps indicate the opening and closing of single channels. Dashed line: zero current level. The all points current histograms on the right-hand side reveal a conductance of ˜50 pS.

FIG. 12. Macroscopic properties of junctions in cell pairs between a hMSC and HeLa cell expressing only Cx40, Cx43 or Cx45. In all cases hMSC to Hela cell coupling was tested 6 to 12 after hours initiating co-culture. A, Ij elicited in response to a series of 5-s voltage steps (Vⱼ) in hMSC-HeLaCx43 pairs. Top, symmetrical current deactivation; bottom, asymmetrical current voltage dependence. B, Macroscopic Ij recordings from hMSC-HelaCx40 pairs exhibit symmetrical (top panel) and asymmetrical (bottom panel) voltage dependent deactivation. C, Asymmetric Ij from hMSC-HeLaCx43 pair exhibits voltage dependent gating when Cx45 side is relatively negative. Ij recorded from hMSC. D, g_{j,ss} plots versus Vⱼ from pairs between hMSC and transfected HeLa cells. Left panel, hMSC-HeLaCx43 pairs, quasi-symmetrical relationship (•) and asymmetrical relationship (∘); continuous and dashed lines are Boltzmann fits (see text for details). Middle panel, symmetrical (•) and asymmetrical (∘) relationships from hMSC-HeLaCx40 pairs; the continuous and dashed lines correspond to Boltzmann fits (see text for details). Right panel, asymmetrical relationship from hMSC-HeLaCx45 cell pairs; continuous line, Boltzmann fit for positive Vⱼ (see text for details). E, Cell-to-cell Lucifer Yellow (LY) spread in cell pairs: from an hMSC to an hMSC (upper panel), from a HeLaCx43 to an hMSC (middle panel), and from an hMSC to a HeLaCx43 (bottom panel). In all cases a pipette containing 2 mM LY was attached to the left-hand cell in the whole-cell configuration. Epifluorescent micrographs taken at 12 min after dye injection show LY spread to the adjacent (right-hand) cell. The simultaneously measured junctional conductance revealed gⱼ of ˜13 nS, ˜16 nS, and ˜18 nS of the pairs, respectively. Cell Tracker green was used to distinguish hMSCs from HeLa cells or vice versa in all experiments.

FIG. 13. Macroscopic and single channel properties of gap junctions between hMSC-canine ventricle cell pairs. Myocytes were plated between 12 and 72 h and co-cultured with hMSCs for 6 to 12 h before measuring coupling. A, Localization of Cx43 for hMSC-canine ventricle cell pairs. Most of Cx43 was localized to the ventricular cell ends and a small amount of Cx43 was present along the lateral borders. The intensive Cx43 staining was detected between the end of the rod-shaped ventricular cell (middle cell) and the hMSC (right cell). There is no detectable Cx43 staining between the ventricular cell and the hMSC on the left side. B, Top, phase-contrast micrograph of a hMSC-canine ventricular myocyte pair. Bottom, monopolar pulse protocol (V₁ and V₂) and associated macroscopic junctional currents (I₂) exhibiting asymmetrical voltage dependence. C, Top, multichannel current elicited by symmetrical biphasic 60 mV pulse. Dashed line, zero current level; dotted lines, represent discrete current steps indicative of opening and closing of channels. The current histograms yielded a conductance of ˜40-50 pS. Bottom, multichannel recording during maintained Vⱼ of 60 mV. The current histograms revealed several conductances of 48-64 pS with several events with conductance of 84 pS to 99 pS (arrows) which resemble operation of Cx43, heterotypic Cx40-Cx43 and/or homotypic Cx40 channels.

FIG. 14. Comparison of gating kinetics of mHCN2 and chimeric mHCN212 channels when expressed in neonatal rat ventricular myocytes. Results using mHCN2 (solid squares) and a chimeric mHCN212 channel (solid circles) are shown. Left, Activation kinetics, determined by fitting the early portion of the current traces (after omitting the initial delay) to a single exponential, for hyperpolarizing test potentials to the voltages indicated on the X-axis. Right, Deactivation kinetics, determined by fitting the current trace from depolarizing test potentials to the indicated voltages following a pre-pulse to a negative potential to fully activate the channels. The time constant of the single exponential fit is plotted on the y-axis in each case, illustrating faster kinetics at all voltages for mHCN212 compared to mHCN2.

FIG. 15. Comparison of expression efficiency of mHCN2 and chimeric mHCN212 channels in neonatal rat ventricular myocytes. Left, Mean current density of expressed current for a step to a negative voltage that maximally activates the channels. Right, Plot of voltage dependence of activation.

FIG. 16. Comparison of mHCN212 characteristics expressed in myocytes and stem cells. The current generated from expression of murine HCN212 in neonatal rat ventricular myocytes and human adult mesenchymal stem cells was measured. Left, voltage dependence of activation; Right, kinetics of activation.

FIG. 17. Properties of wildtype mHCN2 and mHCN112 expressed in oocytes. The steady state activation curve (A), activation kinetics (B) and cAMP modulation (C) are depicted.

FIG. 18. Comparison of gating characteristics of HCN2 and chimeric HCN212 channels when expressed in adult human mesenchymal stem cells. Left, Voltage dependence of activation is shifted significantly positive for mHCN212 (solid circles) compared to HCN2 (solid squares). Right, Kinetics of activation at any measured voltage are significantly faster for mHCN212 compared to HCN2.

FIG. 19. Comparison of performance of biological-electronic tandem pacemaker versus electronic-only pacemaker. A, Percent of electronically paced beats occurring in hearts injected with saline and implanted with an electronic pacemaker or injected with mHCN2 in tandem with an electronic pacemaker. In both groups the electronic pacemaker was set at VVI 45 bpm. Throughout the 14 day period the number of beats initiated electronically was higher in the saline-injected group than in the HCN2-injected group (P<0.05) for comparisons at each time point). B, Mean basal heart rate over days 1-7 and 8-14 of groups injected with saline, mHCN2 or mE324A. Rate in the latter two groups was significantly faster than in the saline group (P<0.05).

FIG. 20. Representative trace of interaction between biological and electronic pacemaker components of tandem unit. This animal had been administered mHCN2. There is a smooth transition from biological to electronic pacemaker activity and from electronic back to biological.

FIG. 21. Effects of epinephrine infusion on biological-electronic tandem pacemaker versus electronic-only pacemaker. IV infusions of 1.0, 1.5 and 2.0 ug/kg/min were given on day 14 until there was either a 50% increase in non-electrically driven pacemaker rate, an arrhythmia occurred, or a maximal dose of 2 μg/kg/min was administered for 10 min. A, Effects of epinephrine, 1 μg/kg/min, on ECGs in three representative animals. Note the greatest rate increase in the mE324A-administered animal. B, A 50% increase in heart rate resulting from idioventricular pacemaker function is indicated in grey. In the saline group, the protocol terminated with all animals having either <50% increase at the highest dose (75% of animals) or an arrhythmia (25% of animals). In the mHCN2 group, 50% of animals had less than a 50% increase in rate: in one animal infusion was terminated because the highest dose was achieved whereas two animals developed ventricular arrhythmias. Of the other 50%, one achieved the 50% rate increase at the lowest epinephrine dose and the other two required 1.5 or 2 μg/kg/min. In contrast, in the mE324A group, 100% achieved a 50% increase in rate at the lowest epinephrine dose and no arrhythmias were seen.

FIG. 22. Comparison of mHCN2 and chimeric mHCN212 provided to rat myocytes in an adenoviral vector. mHCN212 demonstrated a higher basal signal frequency than HCN2, and a less negative maximum diastolic potential.

FIG. 23. Autonomic responsiveness of mHCN2 and HCN212 in newborn rat myocytes. mHCN212 exhibits autonomic responsiveness, demonstrated by an increased signal frequency after exposure to isoproterenol (a beta adrenergic receptor agonist).

FIG. 24. Expression of mHCN212 in human mesencymal stem cells. Panel A shows that hMSCs are expressing GFP, which was co-expressed with mHCN212. GFP is seen in the slides. An electrical potential was applied to the cells following the voltage protocol shown in Panel B. Panel C shows that the current response was blocked, as expected, by cesium.

FIG. 25. Activation of expressed mHCN212 in human mesenchymal stem cells (MSCs). Panel A shows that the amount of current varies with the amount of electrical potential applied. Panel B shows the relationship between the voltage applied and the current generated.

FIG. 26. cAMP modulation of expressed mHCN212 in human mesenchymal stem cells. For a given electrical potential, cAMP will increase the current response. A positive shift for voltage dependence is seen in the presence of cAMP, which indicates a good autonomic responsiveness.

FIG. 27. Expression of mHCN212 in human mesenchymal stem cells provides a higher current density than mHCN2. “n” equals the number of cells tested.

FIG. 28. Characteristics of a biological pacemaker. mHCN2 and mHCN212 express current density (Panel A and B, respectively). Panel C shows that mHCN212 has a more positive current response to an applied electrical potential than mHCN2. Panels D and E show kinetics and demonstrate that HCN212 has faster kinetics than HCN2.

FIG. 29. hMSCs expressing HCN2 provide pacemaker current to generate a stable heart beating rate by day 12-14 after implant. As the number of hMSCs loaded with HCN2 increases, so does the rate. A steady state is reached above roughly 500,000 hMSCs

FIG. 30. Percent of beats triggered by a electronic pacemaker decreased as a function of biological pacemaking by hMSCs on days 12-42 after implant. Dogs were implanted with hMSCs expressing mHCN2. The electronic pacemaker was set to fire when the heart rate fell below 35 beats per minute. As demonstrated in the figure, the number of beats triggered by the electronic pacemaker decreased with implantation of a biological pacemaker comprising about 700,000 hMSCs engineered to express mHCN2.

### DETAILED DESCRIPTION OF THE INVENTION

The present invention relates to the generation of biological pacemakers with desirable clinical characteristics based on expression of wild-type, mutant and chimeric HCN genes (with or without MiRP1 genes or mutants thereof), and the generation of a bypass tract of cells and the use of these biological pacemakers and/or bypass tracts in tandem with electronic pacemakers to create a more effective treatment for cardiac conditions as compared to treatment with biological or electronic pacemakers used alone.

### Biological Pacemakers

A “biological pacemaker” shall mean a biological material such as cell that expresses or is capable of causing the expression of a gene such as an HCN ion channel gene, wherein introduction of this biological material into a heart generated an effective biological pacemaker activity in the heart. “Biological pacemaker activity” shall mean the rhythmic generation of an action potential originating from the introduction of biological material in a cell or a syncytial structure comprising the cell. A “syncytium” or “syncytial structure” shall mean a tissue in which there is gap junction-mediated continuity between the constituent cells. “Inducing or generating a current in a cell” shall mean causing a cell to produce an electric current. An “ion channel” shall mean a channel in a cell membrane created by polypeptide or a combination of polypeptides that localizes to a cell membrane and facilitates the movement of ions across the membrane, thereby generating a transmembrane electric current. An “ion channel gene” shall mean a polynucleotide that encodes a subunit of an ion channel, or more than one subunits thereof or an entire ion channel. A “pacemaker current” shall mean a rhythmic electric current generated by a biological material or electronic device.

As a therapeutic solution, a biological pacemaker can be used to generate a spontaneous beating rate within a physiologically acceptable range that originates from its site of implantation in the heart. “Beating rate” shall mean (1) the contraction rate of heart/myocardium, a portion thereof, or an individual myocyte contraction or contractions over a given time period by a cell (e.g., number of contractions or beats per minute), or (2) the rate of production of an electrical pulse or electrical pulses over a given time period by a cell. This can be achieved by either increasing the rate of a normally spontaneous, but too slowly firing, locus of cardiac cells or by initiating spontaneous activity in a normally quiescent region. Since impulse initiation by a native biological pacemaker relies on the balance between a number of ion channels and transporters, many of which are hormonally modulated, there are several possible approaches to creating a biological pacemaker.

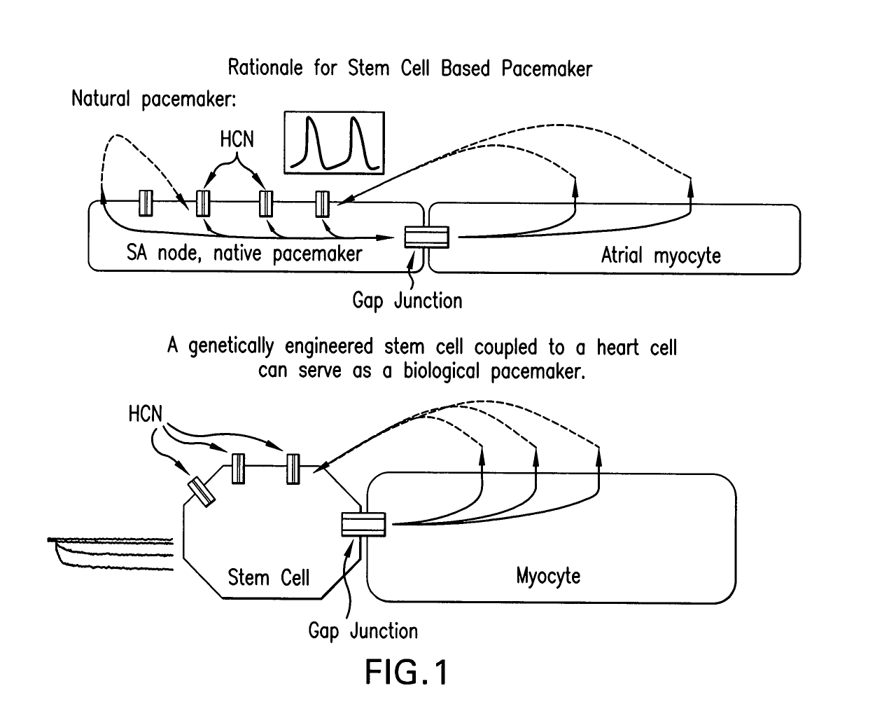

These approaches include, but are not limited to, over-expression of beta-2 adrenergic receptors to increase endogenous atrial rates (Edelberg et al., 1998; 2001), expression of dominant negative Kir2.1 AAA constructs together with the wild-type Kir2.1 gene to suppress the inward rectifier current, I_{K1} (Miake et al., 2002; 2003), over expression of HCN2 channels to increase hyperpolarization-activated, inward pacemaker current (I_{f}) and hence the rate of impulse initiation (Qu et al., 2003; Plotnikov et al., 2004; Potapova et al., 2004), and creating new pacemaker cells from embryonic or mesenchymal stem cells (Kehat et al., 2004; Xue et al., 2005). These approaches seek to manipulate the basic determinants of native pacemaker function in normal hearts; that is, any intervention that increases sympathetic input, decreases repolarizing current, and/or increases depolarizing current during diastole should increase the rate of impulse initiation (Biel et al., 2002). Methods used to achieve these ends have involved gene transfer via viral infection or naked plasmid transfection (Edelberg et al., 1998; 2001), use of embryonic stem cells incorporating a complement of native genes (Kehat et al., 2004), or adult mesenchymal stem cells (MSCs) engineered as platforms to carry pacemaker genes (Potapova et al., 2004). The philosophy behind the latter approach is illustrated in FIG. 1 (a human adult mesenchymal stem cell was engineered to express HCN channels in its cell membrane and was thus able to initiate and propagate an action potential to coupled myocytes through gap junctions). The reproduction of pacemaker action potentials in non-cardiac cells, and/or inducing fusion of non-cardiac and cardiac cells, have also been recently attempted (Cho et al., 2005).

When choosing a strategy for biological pacemakers, the potential for arrhythmogenesis must be considered. The ideal approach would create or enhance spontaneous activity without undesired side effects. In this regard, enhancing autonomic responsiveness by the upregulation of β-adrenergic receptors poses the problem of specificity, since an increase in sympathetic tone is not specific to a single ion current. The targeting of specific ion currents, either by reducing the hyperpolarizing inward rectifier current I_{K1} or enhancing the inward pacemaker current I_{f} both result in increased net inward current in the pacemaker range of potentials. However, I_{K1} also contributes to terminal repolarization, and its down-regulation results in a prolonged action potential (Miake et al., 2002), which has attendant arrhythmic possibilities. By contrast, I_{f} flows only at diastolic potentials and should not affect action-potential duration. Consequently, I_{f} is an attractive molecular target and is preferred for developing biological pacemakers.

Previous studies have focused on the hyperpolarization-activated, cyclic nucleotide-gated (HCN) isoforms responsible for the pacemaker current (“I_{f}”)(Biel et al., 2002) for two reasons: first, the HCN ion current channels initiate pacemaker activity in the mammalian heart; and second, activation of these channels is increased by catecholamines and slowed by acetylcholine, making them autonomically responsive. Autonomic responsiveness should clearly be a cornerstone of pacemaker activity in the heart; yet, lack of this is a key shortcoming of electronic pacemakers.

Hyperpolarization-activated cation currents, termed I_{f}, Iₕ, or I_{q}, were initially discovered in heart and nerve cells over 20 years ago (for review, see DiFrancesco, 1993; Pape, 1996). These currents, carried by Na⁺ and K⁺ ions, contribute to a wide range of physiological functions, including cardiac and neuronal pacemaker activity, the setting of resting potentials, input conductance and length constants, and dendritic integration (see Robinson and Siegelbaum, 2003; Biel et al., 2002). The HCN gene family encodes the channels that underlie the current, and the molecular components of the channels present a natural target for modulating heart rate. The HCN family of ion channel subunits has been identified by molecular cloning (for review, see Clapham, 1998; Santoro and Tibbs, 1999; Biel et al., 2002), and when heterologously expressed, each of the four different HCN isoforms (HCN1-4) generates channels with the principal properties of native I_{f}, confirming that HCN channels are the molecular correlate of this current.

The different HCN isoforms show distinct biophysical properties. For example, in cell-free patches from Xenopus oocytes, the steady-state activation curve of HCN2 channels is 20 mV more hyperpolarized that that of HCN1. Also, whereas the binding of cAMP to a carboxy-terminal cyclic nucleotide binding domain (CNBD) markedly shifts the activation curve of HCN2 by 17 mV to more positive potentials, the response of HCN1 is much less pronounced (4 mV shift).

As such, the present invention provides a tandem pacemaker system comprising (1) an electronic pacemaker, and (2) a biological pacemaker, wherein the biological pacemaker comprises an implantable cell that functionally expresses a hyperpolarization-activated, cyclic nucleotide-gated (HCN) ion channel at a level effective to induce pacemaker current in the cell, when the cell is implanted into a subject's heart. To “functionally express” a nucleic acid shall mean to introduce the nucleic acid into a cell in such a manner as to permit the production of a functional polypeptide encoded by the nucleic acid, so as to thereby produce the functional polypeptide. The encoded polypeptide itself may also be said to be functionally expressed.

A “HCN channel” shall mean a hyperpolarization-activated, cyclic nucleotide-gated ion channel responsible for the hyperpolarization-activated cation currents that are directly regulated by cAMP and contribute to pacemaker activity in heart and brain. There are four HCN isoforms: HCN1, HCN2, HCN3 and HCN4. All four isoforms are expressed in brain; HCN1, HCN2 and HCN4 are also prominently expressed in heart, with HCN4 and HCN1 predominating in sinoatrial node and HCN2 in the ventricular specialized conducting system. “mHCN” designates murine or mouse HCN; “hHCN” designates human HCN. The HCN channel may be any HCN channel that is capable of inducing biological pacemaker activity. “Inducing biological pacemaker activity” in a heart or selected site therein shall mean causing the heart or site therein to rhythmically generate an action potential.

The HCN channel may include, but is not limited to, a naturally occurring HCN channel (from humans and other species), a chimeric HCN channel, a mutant HCN channel, and a chimeric-mutant HCN channel, which are described below.

Biological Pacemakers with HCN Channels

U.S. Pat. No. 6,849,611 teaches an HCN ion channel-containing composition administered to a subject that functions as a site of impulse initiation where sinus node activity is abnormal, thus acting as a biological pacemaker to account for the deficit in the sinus node. U.S. Pat. No. 6,783,979 teaches vectors comprising nucleic acids encoding HCN ion channels that can be applied to a heart tissue so as to provide an ion current in the heart biological tissue. Appropriate administration of such vector can provide expression of the HCN channels to thus in turn generate currents to act as biological pacemakers. The entire contents of the above patents are incorporated herein by reference in their entireties. Also described in U.S. Pat. No. 6,783,979 are biological pacemakers based on expression of HCN genes in combination with MiRP 1. Experiments to generate biological pacemaker activity have been focused on HCN2 because its kinetics are more favorable than those of HCN4, and its cAMP responsiveness is greater than that of HCN1.

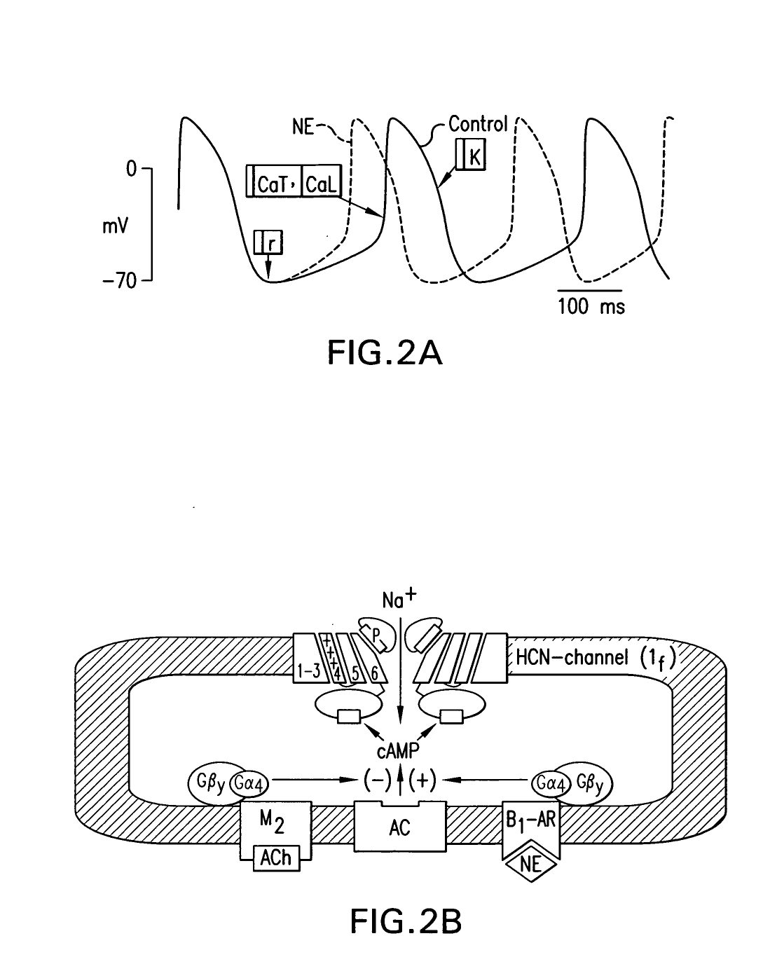

FIG. 2 provides a starting point for understanding the role of HCN channels and the I_{f} current they carry in initiating the pacemaker potential. In brief, phase 4 depolarization is initiated by inward sodium current activated on hyperpolarization of the cell membrane and is continued and sustained by other four major currents (Biel et al., 2002). The major currents provide a balance between inward currents carried by the calcium channel and the sodium/calcium exchanger and outward currents carried by potassium. Activation of the pacemaker potential is increased by β-adrenergic catecholamines and reduced by acetylcholine through their respective G protein-coupled receptors and the adenylyl cyclase-cAMP second messenger system.

Full-length cDNAs encoding HCN1-4 isoforms have been cloned from different species and have been functionally characterized following expression in mammalian cell lines. See, for example, Santoro et al. (1998) and Ludwig et al. (1998) reporting the cloning and functional characterization of HCN1-3 from mouse brain; Ludwig et al. (1999) reporting the cloning and functional characterization of HCN2 and HCN4 from human heart; Ishii et al. (1999) reporting the cloning and functional characterization of HCN4 from rabbit heart; Monteggia et al. (2000) reporting the cloning of HCN1-4 in rat brain; and Steiber et al. (2005) reporting the cloning and functional characterization of HCN3 from human brain.

The amino acid identity between different HCN isoforms in a species varies from about 45-60%, with differences primarily due to low sequence identity in the N- and C-terminal regions. For example, the primary sequences of mHCN1-3 have an overall amino acid identity of about 60% (Ludwig et al., 1999), and hHCN3 has 46-56% homology with the other hHCNs (Stieber et al., 2005). By comparison, significantly higher degrees of homology have been observed between cognate isoforms in different species. For example, Ludwig et al. (1999) report that the hHCN2 cDNA clone has 94% overall sequence identity with a mHCN2 clone; Stieber et al. (2005) report that hHCN3 has 94.5% amino acid homology with mHCN3; and in a review on HCN channels, Biel et al. (2002) disclose that the primary sequences of individual HCN channel types exhibit over 90% sequence identity in mammals.

Table 1, adapted from Stieber et al. (2005), Supplement Table S2, shows the amino acid homology of hHCN3 with the other hHCNs and with mHCN3. Particularly striking is the near-100% homology of the hHCN3 and mHCN3 sequences in the core transmembrane domains and the cyclic nucleotide binding domain. The N-terminal and C-terminal regions of hHCN3 and mHCN3 are 81 and 91% homologous, respectively, which are lower than the degree of homology in the transmembrane and CNDB regions, but still considerable higher than the 22-35% homology between the N-terminus of hHCN3 and the N-terminal regions of other hHCN isoforms, 17-27% homology in the C-terminal regions, and 46-56% overall homology between hHCN3 and other hHCN isoforms.

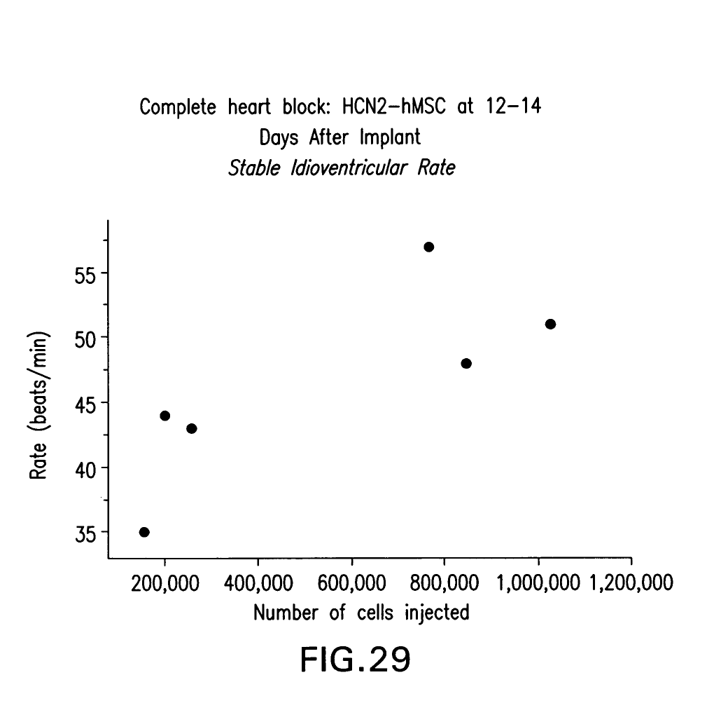

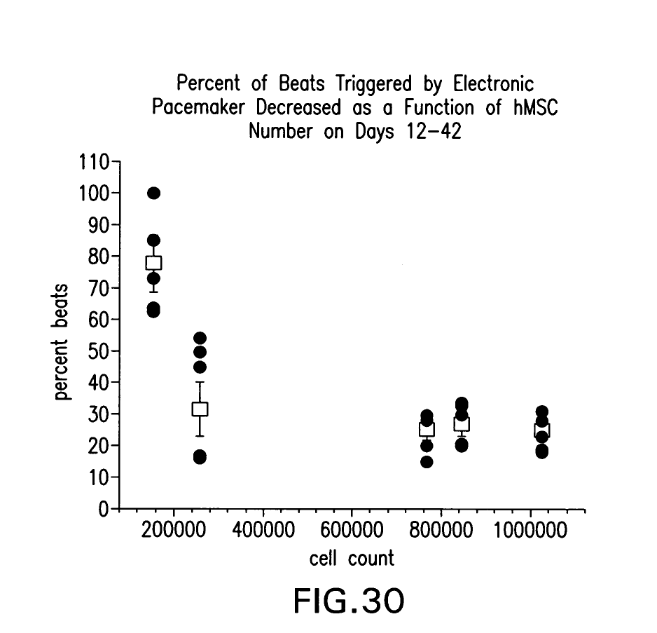

These homology data suggest that cognate HCN isoforms from different species can be effectively substituted in the present invention; for example, hHCN2 or portions thereof can be substituted for mHCN2 or corresponding portions thereof. Accordingly, in the present invention, a biological pacemaker or method comprising the use of HCN2 or portions thereof from one species, for example mouse, encompasses the use of HCN2 or corresponding portions thereof from other species, preferably mammalian species, including, but not limited to, a human, rat, dog, rabbit, or guinea pig. See FIGS. 29 and 30 and Examples 3 and 5 where mHCN2 was used in a canine to generate a pacemaker signal, thus showing the interchangeability of the isoforms between species. Similarly, a biological pacemaker or method comprising the use of mouse HCN1, HCN3 or HCN4 or portions thereof encompasses the use of HCN1, HCN3, or HCN4, or corresponding portions thereof, respectively, from other species, preferably other mammalian species.

More generally, a biological pacemaker or method comprising the use of a particular HCN isoform encompasses the use of an HCN channel exhibiting at least 75%, preferably at least 85%, more preferably at least 90%, and most preferably at least 95% overall homology with that isoform. In embodiments of the invention comprising portions of an HCN isoform, the use of a N-terminal portion of a particular HCN isoform encompasses the use of a N-terminal portion of a HCN channel exhibiting at least 60%, preferably at least 70%, more preferably at least 80% homology with the N-terminus of that isoform. In addition, the use of a C-terminal portion of a particular HCN isoform encompasses the use of a C-terminal portion of a HCN channel exhibiting at least 60%, preferably at least 70%, more preferably at least 80%, and most preferably at least 90% homology with the C-terminus of that isoform.

TABLE 1
Amino Acid Homology between hHCN3 and hHCN1,
2 and 4 and mHCN3

| Amino acid homology¹ | hHCN1 | hHCN2 | hHCN4 | mHCN3 |
|---|---|---|---|---|
| compared to hHCN3 | (%) | (%) | (%) | (%) |
| Overall | 53.0 | 55.8 | 45.7 | 94.5 |
| N-terminus | 34.6 | 28.4 | 22.2 | 80.7 |
| S1 | 78.3 | 78.3 | 87.0 | 100 |
| S1-S2 linker | 64.3 | 71.4 | 78.6 | 100 |
| S2 | 77.3 | 90.9 | 90.9 | 100 |
| S2-S3 linker | 41.7 | 54.2 | 50.0 | 100 |
| S3 | 84.2 | 79.0 | 84.2 | 100 |
| S3-S4 linker | 36.4 | 36.4 | 45.5 | 100 |
| S4 | 100 | 100 | 100 | 100 |
| S4-S5 linker | 100 | 94.4 | 100 | 100 |
| S5 | 96.0 | 92.0 | 96.0 | 100 |
| S5 linker-Pore-S6 linker | 82.0 | 77.6 | 85.7 | 93.9 |
| S6 | 89.7 | 96.6 | 100 | 100 |
| S6-CNBD linker | 82.9 | 85.4 | 91.5 | 100 |
| CNBD² | 78.3 | 80.0 | 80.8 | 99.2 |
| C-terminus | 17.4 | 26.5 | 19.1 | 90.7 |

¹For this comparison, identical and similar amino acids are considered homologous.
²Cyclic nucleotide binding domain

Percentage “homology” between peptide sequences shall mean the degree, expressed as a percentage, to which the amino acid residues at equivalent positions in the peptides, when aligned for maximum correspondence, are identical or functionally similar. Examples of functionally similar amino acids include glutamine and asparagine; serine and threonine; and valine, leucine and isoleucine. Percentage “amino acid identity” or percentage “sequence identity” between peptide sequences shall mean the degree, expressed as a percentage, to which the amino acid residues at equivalent positions in the peptides, when aligned for maximum correspondence, are identical. For peptides, the percentage homology is usually greater than the percentage sequence identity. For nucleic acids, percentage “homology” shall mean the same as percentage “sequence identity”, which is the degree, expressed as a percentage, to which the nucleotides at equivalent positions in the nucleic acids, when aligned for maximum correspondence, are identical.

For the purpose of the invention, two sequences that share homology, i.e., a desired polynucleotide and a target sequence, may hybridize when they form a double-stranded complex in a hybridization solution of 6×SSC, 0.5% SDS, 5× Denhardt's solution and 100 g of non-specific carrier DNA. See Ausubel et al., section 2.9, supplement 27 (1994). Such sequence may hybridize at “moderate stringency,” which is defined as a temperature of 60° C. in a hybridization solution of 6×SSC, 0.5% SDS, 5× Denhardt's solution and 100 μg of non-specific carrier DNA. For “high stringency” hybridization, the temperature is increased to 68° C. Following the moderate stringency hybridization reaction, the nucleotides are washed in a solution of 2×SSC plus 0.05% SDS for five times at room temperature, with subsequent washes with 0.1×SSC plus 0.1% SDS at 60° C. for 1 h. For high stringency, the wash temperature is increased to typically a temperature that is about 68° C. Hybridized nucleotides may be those that are detected using 1 ng of a radiolabeled probe having a specific radioactivity of 10,000 cpm/ng, where the hybridized nucleotides are clearly visible following exposure to X-ray film at −70° C. for no more than 72 hours.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences that may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

Biological Pacemakers with Chimeric HCNs

The present invention also provides biological pacemakers comprising an implantable cell that functionally expresses a chimeric HCN at level effective to generate an effective pacemaker current in the cell, when the cell is implanted into a subject's heart, and the use thereof in a tandem pacemaker system.

A “HCN chimera” shall mean an HCN ion channel comprising portions of more than one type of HCN channel. For example, a chimera may comprise portions of HCN1 and HCN2 or HCN3 or HCN4, and so forth. In addition, an ion channel chimera shall mean an ion channel comprising portions of an HCN channel derived from different species. For example, one portion of the channel may be derived from a human and another portion may be derived from a non-human.

The term “HCNXYZ” (wherein X, Y and Z are any one of the integers 1, 2, 3 or 4, with the proviso that at least one of X, Y and Z is a different number from at least one of the other numbers) shall mean a chimeric HCN channel polypeptide comprising three contiguous portions in the order XYZ, wherein X is an N-terminal portion, Y is an intramembrane portion, and Z is a C-terminal portion, and wherein the number X, Y or Z designates the HCN channel from which that portion is derived. For example, HCN112 is an HCN chimera with a N-terminal portion and intramembrane portion from HCN1 and a C-terminal portion from HCN2.

Wang et al. (2001b) used chimeras between HCN1 and HCN2 to investigate the molecular bases for the modulatory action of cAMP and for the differences in the functional properties of the two channels. The present invention discloses manipulation of the properties of HCN channels by in vitro recombination of nucleotide sequences encoding portions of all four HCN isoforms to produce HCN chimeras. As detailed in the example 4, certain of these chimeras, such as HCN212, exhibit characteristics which are advantageous for generating pacemaker currents in treating heart disorders.

In general terms, HCN polypeptides can be divided into three major domains: (1) a cytoplasmic amino terminal domain; (2) the membrane spanning domains and their linking regions; and (3) a cytoplasmic carboxy-terminal domain. To date, there is no evidence that the N-terminal domain plays a major role in channel activation (Biel et al., 2002). As described herein, the membrane spanning domains with their linking regions play an important role in determining the kinetics of gating, whereas the CNBD in the C-terminal domain is in large part responsible for the ability of the channel to respond to the sympathetic and parasympathetic nervous systems that respectively raise and lower cellular cAMP levels. One skilled in the art would be able to determine which amino acids of an HCN polypeptide comprise the amino terminal domain, the membrane spanning domains, their linking regions and the cytoplasmic carboxy-terminal domain.

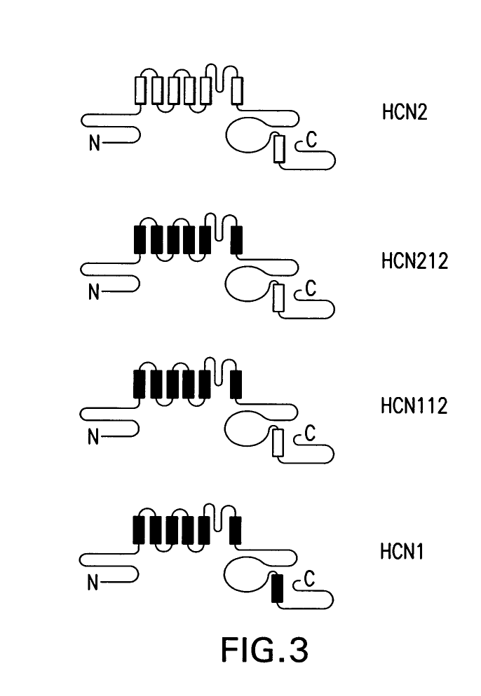

Preferred embodiments of the present invention provide pacemaker systems comprising cells expressing chimeric HCN channels that provide fast kinetics and good responsiveness to cAMP. HCN1 has the fastest kinetics but poor cAMP responsiveness. HCN2 has slower kinetics and good cAMP responsiveness. Accordingly, chimeras of HCN1 and HCN2 were studied experimentally and the invention provides pacemaker systems comprising cells expressing these and other chimeras. A schematic representation of HCN1/HCN2 chimeras is shown in FIG. 3.

In some embodiments of the biological pacemakers of the present invention, the HCN chimera comprises an amino terminal portion contiguous with an intramembrane portion contiguous with a carboxy terminal portion, wherein each portion is a portion of an HCN channel or a portion of a mutant thereof, and wherein one portion derives from an HCN channel or a mutant thereof which is different from the HCN channel or mutant thereof from which at least one of the other two portions derive. In a further embodiment, at least one portion of the HCN chimera is derived from an animal species which is different from the animal species from which at least one of the other two portions derive. For example, one portion of the channel may be derived from a human and another portion from a non-human. In one embodiment, the intramembrane portion is D129-L389 of mHCN1. In other embodiments, the chimeric polypeptide comprises mHCN112, mHCN212, mHCN312, mHCN412, mHCN114, mHCN214, mHCN314, mHCN414, hHCN112, hHCN212, hHCN312, hHCN412, hHCN114, hHCN214, hHCN314, or hHCN414.

In different embodiments, the HCN chimera is mHCN112, mHCN212, mHCN312, mHCN412, mHCN114, mHCN214, mHCN314, mHCN414, hHCN112, hHCN212, hHCN312, hHCN412, hHCN114, hHCN214, hHCN314, or hHCN414. In a preferred embodiment, the HCN chimera is hHCN112 or hHCN212.

The HCN112 chimera (containing the N-terminal domain of HCN1, membrane spanning domains of HCN1, and C-terminal domain of HCN2) is a preferred candidate channel for biological pacemaking because it contains the relevant membrane spanning domains of HCN1 (exhibiting fast kinetics) and the C-terminal domain of HCN2 (exhibiting good cAMP responsiveness). See FIG. 3. HCN212 is also a preferred candidate. See FIG. 3. Other preferred chimeras are HCN312 and HCN412. HCN4 also exhibits slow kinetics and good cAMP responsiveness; thus, HCN114, HCN214, HCN314 and HCN414 are desirable chimeras.

Whereas the HCN channels are defined above in terms of three broad functional domains, there are multiple locations at which the borders between these domains in a chimeric channel could be set. The present invention also encompasses variants of HCN chimeras created using domains with differently defined boundaries which also serve to recombine the desirable biochemical and biophysical characteristics of individual HCN channels.

In preferred embodiments the chimeric HCN channel provides an improved characteristic, as compared to a wild-type HCN channel, including, but not limited to, faster kinetics, more positive activation, increased expression levels, increased stability, enhanced cAMP responsiveness, and enhanced neurohumoral response.

Biological Pacemakers with Mutant HCNs

The present invention also provides biological pacemakers comprising an implantable cell that functionally expresses a mutant HCN when implanted into a subject a level effective to induce an effective pacemaker current in the cell, and the use of thereof in a tandem pacemaker system.

Most of what is known about voltage activation of ion channels comes from studies of voltage-gated K⁺ (Kv) channels. Although HCN channels open in response to membrane hyperpolarization instead of depolarization as in Kv channels, HCN channels have a transmembrane topology that is highly similar to Kv channels. All of these ion channels have four subunits, each of which has six transmembrane segments, S1-S6: the positively charged S4 domain forms the major voltage sensor, whereas S5 and S6, together with the S5-S6 linker connecting the two, form the pore domain containing the ion permeation pathway and the gates that control the flow of ions (Larsson, 2002). The activation gate is formed by the crossing of the C-terminal end of the S6 helices (Decher et al., 2004). Much progress has been made, based on biophysical experiments and the recently described structures of bacterial K⁺ channels, in understanding the physical basis for the activation and inactivation of gates, selective ion permeability, and voltage sensing mechanisms of ion channels. However, the molecular mechanisms whereby changes in voltage open and close these channels, and the coupling mechanisms between the voltage sensors and the gates, are still largely not understood. In particular, it remains unclear how the coupling mechanism results in opposite voltage dependence of activation for Kv and HCN channels.

Coupling of the movement of the voltage sensor to the opening and closing of the HCN channel pore could involve global rearrangements of the S4, S5 and S6 transmembrane domains without the need for specific amino acid interactions. However, recent studies suggest that physical coupling may include specific interactions between amino acids in the S4S5 linker and the S6 domain (Chen et al., 2001a; Decher et al., 2004). These studies suggest that the S4-S5 linker is an important component of the coupling mechanism that mediates the hyperpolarization-activated opening of HCN channels.

Voltage sensing and activation of HCN channels can be altered by mutation. For example, alanine-scanning mutagenesis of the S4-S5 linker in HCN2 revealed that three amino acids were especially critical for normal gating (Chen et al., 2001a). Mutation of Y331 or R339, and to a lesser extent, E324, disrupted channel closure. Mutation of a basic residue in the S4 domain (R318Q) prevented channel opening. Conversely, channels with R318Q and Y331S double mutations were constitutively open. Using alanine-scanning mutagenesis of the C-terminal end of S6 and the C-linker that connects S6 to the CNBD, Decher et al. (2004) identified five residues that were important for normal gating as mutations disrupted channel closure. Further mutation analyses suggested that a specific electrostatic interaction between R339 of the S4-S5 linker and D443 of the C-linker stabilizes the closed state and thus participates in the coupling of voltage sensing and activation gating in HCN channels. Interactions between residues in the S4-S5 linker and the C-terminal end of the S6 domain have also been shown to be critical for stabilizing hERG and ether-á-go-go channels in a closed state (Ferrer et al., 2006). These mutation studies indicate that mutations in the S4 voltage sensor, the S4-S5 linker implicated in the coupling of voltage sensing to pore opening and closing, the S5, S6 and S5-S6 linker which form the pore, the C-linker, and the CNBD, may be particularly important in affecting HCN channel activity.

Accordingly, the present invention provides a biological pacemaker, wherein the biological pacemaker comprises an implantable cell that functionally expresses a mutant HCN ion channel when implanted in a subject at a level effective to induce effective pacemaker current in the cell. In preferred embodiments the mutant HCN channel provides an improved characteristic, as compared to a wild-type HCN channel, including, but not limited to, faster kinetics, more positive activation, increased expression levels, increased stability, enhanced cAMP responsiveness, and enhanced neurohumoral response. In certain embodiments of the present invention, the mutant HCN channel carries at least one mutation in S4 voltage sensor, the S4-S5 linker, S5, S6, the S5-S6 linker, and/or the C-linker, and the CNBD, which mutations result in one or more of the above discussed characteristics. In other embodiments, the HCN mutant is E324A-HCN2, Y331A-HCN2, R339A-HCN2, or Y331A,E324A-HCN2. In a preferred embodiment, the mutant HCN channel is E324A-HCN2.

In addition to the mutations noted above, many mutations in different HCN isoforms have been reported. These include R318Q, W323A, E324A, E324D, E324K, E324Q, F327A, T330A and Y331A, Y331D, Y331F, Y331K, D332A, M338A, R339A, R339c, R339D, R339E and R339Q in HCN2 made by Chen et al. (2001a) to investigate in greater detail the role of the E324, Y331 and R339 residues in voltage sensing and activation. Chen et al. (2001b) have also reported the R538E and R591E mutations in mHCN1; Tsang et al. (2004) have reported G231A and M232A in mHCN1; Vemana et al (2004) have reported R247c, T249C, K250C, 1251C, L252C, S253C, L254C, L258C, R259c, L260C, S261C, C318S, S338C in mHCN2; Macri and Accili (2004) have reported S306Q, Y331D AND G404S in mHCN2; and Decher et al. (2004) have reported Y331A, Y331D, Y331S, R331FD, R339E, R339Q, 1439A, S441A, S441T, D443A, D443C, D443E, D443K, D443N, D443R, R447A, R447D, R447E, R447Y, Y449A, Y449D, Y449F, Y449G, Y449W, Y453A, Y453D, Y453F, Y453L, Y453W, P466Q, P466V, Y476A, Y477A and Y481A in mHCN2. The entire contents of all of the above publications are incorporated herein by reference. Certain of the reported mutations listed above may confer, singly or in combination, beneficial characteristics on the HCN channel with regard to creating a biological pacemaker. The invention disclosed herein encompasses all mutations in HCN channels, singly or in combinations, which improve pacemaker activity of the channel such as by providing faster kinetics, more positive activation, increased expression and/or stability, enhanced cAMP responsiveness, and/or enhanced neurohumoral response.

Mutations are identified herein by a designation with provides the single letter abbreviation of the amino acid residue that underwent mutation, the position of that residue within a polypeptide, and the single letter abbreviation of the amino acid residue to which the residue was mutated. Thus, for example, E324A identifies a mutant polypeptide in which the glutamate residue (E) at position 324 was mutated to alanine (A). Y331A, E324A-HCN2 indicates a mouse HCN2 having a double mutation, one in which tyrosine (Y) at position 331 was mutated to alanine (A), and the other in which the glutamate residue at position 324 was mutated to alanine.

Experiments disclosed herein have explored the E324A mutation in mHCN2 that has been reported to exhibit both faster kinetics and a more positive activation relation (Chen et al., 2001a). Both these characteristics should enhance pacemaking. Details of the pacemaker activity of E324A compared to HCN2 when expressed in myocytes, Xenopus oocytes, and in situ in dog hearts are provided in the Examples.

Biological Pacemaker with HCN Channels (Including Mutants or Chimeras) With MiRP1

Another approach to enhancing biological pacemaker activity of a HCN channel by increasing the magnitude of the current expressed and/or speeding its kinetics of activation is to co-express HCN2 with its beta subunit, MiRP1. Qu et al. (2004) infected myocyte cultures with a HCN2 adenovirus and a second adenovirus that was a vehicle for either GFP or an HA-tagged form of MiRP1. The result was a significant increase in current magnitude and acceleration of activation and deactivation kinetics. See also U.S. Pat. No. 6,783,979, the entire contents of which are incorporated herein by reference.

Many MiRP1 mutations have been reported (see, e.g., Mitcheson et al., 2000; Lu et al., 2003; Piper et al., 2005), and certain of these mutations, or combinations thereof, may be beneficial in increasing the magnitude and kinetics of activation of the current expressed by a HCN channel used to create a biological pacemaker. The invention disclosed herein encompasses all such mutations, or combinations thereof, in MiRP1.

Cells of the Biological Pacemaker

“Implantable cell” means a cell that can be implanted or administered into a subject. A “cell” shall include a biological cell, e.g., a HeLa cell, a stem cell, or a myocyte, and a non-biological cell, e.g., a phospholipid vesicle (liposome) or virion. Preferably biological pacemakers of the present invention comprise an implantable biological cell capable of gap junction-mediated communication with cardiomyocytes. Exemplary cells include, but are not limited to, a stem cell, a cardiomyocyte, a fibroblast or skeletal cell engineered to express connexins, or an endothelial cell. The stem cell may be an embryonic or adult stem cell substantially incapable of differentiation. In a preferred embodiment the cell is an adult mesenchymal stem cell, and more preferred embodiments, the cell is an adult human mesenchymal stem cell. Experiments described below indicate that hMSCs provide an attractive platform for delivery HCN ion channels into the heart.

In a preferred embodiment, the adult human mesenchymal stem cell has been passaged at least nine times, or in a more preferred embodiment nine to 12 times, expresses CD29, CD44, CD54 and HLA class I surface markers and fails to express CD14, CD45, CD34 and HLA class II surface markers. Such adult human mesenchymal stem cell seem to be substantially incapable of differentiation but yet maintain the markers identifying them as stem cells. See co-pending provisonal application 60/______ (awaited) entitled “Use of late passage mesenchymal (MSCs) for treatment of cardiac disorders” filed on Jul. 21, 2006, concurrently herewith, which is herein incorporated by reference in its entirety.

There have been recent reports of the delivery of bone marrow-derived and/or circulating hMSCs to the hearts of post-myocardial infarct patients resulting in some improvement of mechanical performance (Strauer et al., 2002; Perin et al., 2003) in the absence of overt toxicity. The presumption in these and other animal studies (Orlic et al., 2001) is that the hMSCs integrate into the cardiac syncytium and then differentiate into new heart cells restoring mechanical function. However, no differentiation of hMSCs was seen over a 42-day period following injection of mHCN2-transfected hMSCs into LV subepicardium of 6 non-immunosuppressed adult dogs (Plotnikov et al., 2005b). Moreover, it has been shown that passaging hMSCs 9 times or more, and preferably 9-12 times, prevents differentiation (unpublished data). See co-pending provisional 60/______ (awaited) entitled “Use of late passage mesenchymal (MSCs) for treatment of cardiac disorders” filed concurrently herewith on Jul. 21, 2006.

In preferred biological pacemakers of the present invention and preferred tandem systems comprising the biological pacemaker, the amount of cells to be implanted is an amount that is required to generate an effective pacemaker current. An “effective pacemaker current” means that cells expressing an HCN channel, a chimeric HCN channel or a mutant HCN channel as described above, generate a pacemaker current that is effective to cause the subject's heart to beat. The strength of the pacemaker current or the heart beating rate generated by the pacemaker current need not be at the level of a normal healthy heart, however, in preferred embodiments, the biological pacemaker functions as well as a normal healthy naturally occurring pacemaker.

In certain embodiments, the biological pacemaker comprises between 5,000 to 1.5 million human adult mesenchymal stem cells. In other embodiments the biological pacemaker comprises between about 700,000 to 1.0 million human adult mesenchymal stem cells. In one embodiment, the biological pacemaker comprises at least about 5,000 cells. In a preferred embodiment, the biological pacemaker comprises at least about 200,000 human adult mesenchymal stem cells. In another preferred embodiment the biological pacemaker comprises at least about 500,000 cells. In a more preferred embodiment, the biological pacemaker comprises at least about 700,000 human adult mesenchymal stem cells. See FIGS. 29 and 30.

Delivery of HCN Channels to an Implantable Cell to Create a Biological Pacemaker

To create certain biological pacemakers of the present invention, a nucleic acid encoding an HCN channel as described above, must be delivered to an implantable cell (as described above). Electroporation is a preferred in vitro method for genetically engineering cells such as hMSCs to overexpress I_{f} (HCN channels) for in vivo delivery to a subject's heart. Electroporation is a technique in which exposure of cells to a brief pulse of high voltage transiently opens pores in the cell membranes that allow macromolecules, such as DNA and proteins, to enter the cell. It has been demonstrated that electroporation can also be applied in vivo to deliver nucleic acids and proteins into muscle cells of live animals including rats, mice and rabbits (see U.S. Pat. No. 6,110,161), and the method has been used to deliver DNA directly into embryonic chick heart (Harrison et al., 1998) and into mammalian myocardium prior to transplantation (Wang et al., 2001c).

Other methods of introducing genes into an implantable cell for implantation into the heart include viral transfection using, for example, adenovirus, adeno-associated virus (AAV), and lentivirus, liposome-mediated transfection (lipofection), transfection using a chemical transfection reagent, heat shock transfection, or microinjection. AAV, a small parvovirus associated with adenovirus, cannot replicate on its own and requires co-infection with adenovirus or herpesvirus in order to replicate. In the absence of helper virus, AAV enters a latent phase during which it stably integrates into the host cell genome. This latent phase makes AAV attractive for certain gene therapy applications involving transfer of genes of up to about 4.4 kb, as the gene inserted into AAV can persist in the host cell genome for a long period (Pfeifer and Verma, 2001). Lentivirus, a member of the retroviral family, provides a potentially interesting alternative (Amado and Chen, 1999; Trono, 2002). Unlike adenoviruses, electroporation and the use of lentiviral vectors allow persistent transgene expression without eliciting host immune responses.

Safety is a factor to be demonstrated especially with viral vectors. The absence of arrhythmias and neoplasia generated by viral vectors or cells should be demonstrated along with an absence of infection or engraftment at distant locations. Once safety and efficacy have been demonstrated, cost-effectiveness should also be considered. Even if the problems of expression and delivery are surmounted, long-term persistence of a cell-based pacemaker requires the absence of rejection if nonautologous cells are employed. In this regard, hMSCs could be obtained from an autologous source. However, evidence suggesting that these cells are immunoprivileged (Liechty et al., 2000) may reduce the need for autologous sources. The long-term extent of this privilege has not been tested, but no cellular or humoral rejection was evident six weeks following injection of hMSCs into canine hearts (Plotnikov et al., 2005b). Rejection remains a consideration for embryonic stem cells. Allogeneic solutions based on the immunoprivileged status of hMSCs would provide a more favorable model since off-the-shelf cells could be ready for implantation.

Delivery of Implantable Cells into a Subject's Heart

A cell-based biological pacemaker of the present invention is preferably administered to a selected site in the heart of a subject. Several methods to achieve focal delivery are feasible; for example, the use of catheters and needles, and/or growth on a matrix and a “glue.” Whatever approach is selected, the delivered cells should not disperse to far from the target site. Such dispersion could introduce unwanted electrical effects within the heart or in other organs. It is noteworthy that in a preliminary study involving injection of up to ˜10⁶ HCN2-transfected hMSCs into the LV subepicardium of six adult dogs, nests of hMSCs were consistently found adjacent to the injection site but not at a distance (Plotnikov et al., 2005b).

In various embodiments of the instant pacemaker systems and methods, implantable cells are administered onto or into the heart by injection, catheterization, surgical insertion, or surgical attachment. The delivery site is determined at the time of administration, based on the patient's pathology, to give the optimal activation and hemodynamic response. Thus, the chosen site could be the sinoatrial (SA) node, Bachmann's bundle, the atrioventricular junctional region, His branch, left or right bundle branch, Purkinje fibers, right or left atrial muscle or ventricular muscle, the appropriate site being well known to one of ordinary skill in the art. The isoform or type of HCN ion channel expressed in the heart may also be changed depending on the delivery site. In addition, different levels of expression of the ion channel gene may be desirable in different delivery sites. Such different levels of expression may be obtained by using different promoters to drive expression at a desired level.

In another embodiment, implantable cells are locally administered by injection or catheterization directly onto or into the heart. In further embodiments, the cell is systemically administered by injection or catheterization into a coronary blood vessel or a blood vessel proximate to the heart. In still further embodiments, the cell is injected onto or into an area of an atrium or ventricle of the heart. In other embodiments, the cell is injected onto or into the left atrium, a wall of a ventricle, a bundle branch of a ventricle, or the proximal LV conducting system of the heart.

Biological Pacemaker Formed by Administering Expression Vector with HCN Channel into a Subject's Heart

In certain embodiments of the present invention, the biological pacemaker is formed directly in a subject's heart. In such embodiments, a vector(s) comprising a nucleic acid encoding an HCN channel (including chimeras and mutants) and/or MiRP1 as described above is administered to a cell in the heart of a subject. The vector functionally expresses the HCN channel to generate an effective pacemaker current in the heart as described above.

Vectors comprising the desired nucleic acids (i.e. HCN channel, and MiRP1, etc.) may be any suitable expression vector, including necessary regulatory elements such as promoters, which would provide expression of the nucleic acid. One skilled in the art would appreciate and choose a suitable vector and any other regulatory elements necessary to provide expression of the nucleic acid when administered to a cell in the heart. For example, the vector may be as described above. One skilled in the art would understand and appreciate different methods of administering vectors to cells in a subject. For example, a vector may be administered onto or into the heart by injection or catheterization. In certain embodiments, a vector is administered onto or into the area/region of the heart best situated to treat a subject. One skilled in the art would understand an appropriate administration cite. For example, if the subject to be treated had a normal functioning heart, but a defective sinoatrial node, one might contemplate administering a vector as described above to the subject's sinoatrial node. Exemplary cites for administration include, but are not limited to, the Bachmann's bundle, sinoatrial node, atrioventricular junctional region, His branch, left or right bundle branch, Purkinje fibers, right or left atrial muscle, a wall of a ventricle, or the proximal left ventricular (LV) or right ventricular (RV) conducting system of the heart.

Tandem System with Bypass Bridge

The present invention also provides a tandem pacemaker system comprising (1) an electronic pacemaker, and (2) a bypass bridge comprising a strip of gap junction-coupled cells having a first end and a second end, both ends capable of being attached to two selected sites in a heart, so as to allow transmission of a pacemaker and/or electrical signal/current across the bridge between the two sites in the heart. In certain embodiments, the bypass bridge is an atrioventricular bridge, where the first end of the bypass bridge is capable of being attached to the atrium and the second end is capable of being attached to the ventricle, so as to allow transmission of an electrical signal from the atrium to travel across the tract to excite the ventricle.

Bypass bridges and atrioventricular bridges have been described PCT International Publication No. WO 2005/062857, U.S. Provisional Application No. 60/704,210 (filed Jul. 29, 2005), and U.S. application Ser. No. 10/745,943 (filed Dec. 24, 2003), and U.S. application Ser. No. 11/______ (awaited) entitled “A Biological Bypass Bridge with Sodium Channels, Calcium Channels and/or Potassium Channels to Compensate for Conduction Block in the Heart” filed concurrently herewith on Jul. 21, 2006, the entire contents of which are all incorporated herein by reference.

The tract of gap junction-coupled cells, may be any implantable cell as described above with respect to implantable cells of the biological pacemakers (i.e. stem cells, cardiomyocytes, fibroblast or skeletal muscle cells engineered to express cardiac connexins or endothelial cells). In certain embodiments, the cells functionally express a protein which is a cardiac connexin, an alpha subunit and accessory subunits of a L-type calcium channel, an alpha subunit with or without the accessory subunits of a sodium channel, or a L-type calcium and/or sodium channel in combination with the alpha subunit of a potassium channel, with or without the accessory subunits of the potassium channel. In a further embodiment, the connexin is Cx43, Cx40, or Cx45.

In certain embodiments, the cell is an adult human mesenchymal stem cell (MSC). MSCs may be prepared in several ways including, but not limited to, the following:

1: In culture without incorporation of additional molecular determinants of conduction. Here the cells' own characteristic to generate gap junctions that communicate electrical signals are used as a means to propagate an electronic wave from cell to cell.

2: In culture following electroporation to introduce the gene for connexins 43, 40 and/or 45, to enhance formation of gap junctions and thereby facilitate cell-to-cell propagation of electric signals.

3: In culture following electroporation to introduce genes encoding different types of ion channels, including the alpha and the accessory subunits of a L-type calcium channel, the alpha subunit with or without the accessory subunits of a sodium channel, or the L-type calcium and/or sodium channel in combination with the alpha subunit of a potassium channel, with or without the accessory subunits of the potassium channel. The expression of these ion channels increases the likelihood of not just electrotonic propagation of a wavefront, but its active propagation by an action potential.

4: A combination of methods 2 and 3. These hMSCs thus produced are grown in culture on a non-bioreactive material. The hMSCs will couple together via gap junctions, as described herein.

In certain embodiments, when the bypass bridge is an atrioventricular bridge, once growth is complete, one end of the bridge is sutured to the atrium, and the other to the ventricle. Electrical signals generated by the sinus node to activate the atria will propagate across the artificially constructed tract to excite the ventricle as well. In this way the normal sequence of atrioventricular activation will be maintained.

The preparation of an atrioventricular bypass in this fashion not only facilitates propagation from atrium to ventricle, but also provides sufficient delay from atrial to ventricular contraction to maximize ventricular filling and emptying and mimic the normal activation and contractile sequence of the heart.

In certain embodiments, the present invention provides a tandem pacemaker system comprising an electronic pacemaker and a bypass bridge and further comprises a biological pacemaker, preferably the biological pacemakers of the present invention. In preferred embodiments, the bypass bridge is an atrioventricular bridge.

As described above, the tandem system comprises an electronic pacemaker. Electronic pacemakers are known in the art. Exemplary electronic pacemakers are described in U.S. Pat. Nos. 5,983,138, 5,318,597 and 5,376,106; Hayes (2000); and Moses et al. (2000), the entire contents of all of which are incorporated herein by reference. The subject may have already been fitted with an electronic pacemaker or may be fitted with one simultaneously or after placement of the biological pacemaker. The appropriate site for the electronic pacemaker would be well known to a skilled practitioner, depending on the subject's condition and the placement of the biological pacemaker of the present invention. For example, if the subject had a functional sinoatrial node, but had a block between the sinoatrial node and the atrioventricular node, the biological pacemaker might preferably be administered to the atrioventricular node. Preferred insertion cites include, but are not limited to, the Bachmann's bundle, sinoatrial node, atrioventricular junctional region, His branch, left or right bundle branch, Purkinje fibers, left or right atrial muscle or ventricular muscle of the subject's heart.

In preferred embodiments of the present invention, the electronic pacemaker is programmed to produce its pacemaker signal on an “as-needed” basis, i.e., to sense the biologically generated beats and to discharge electrically when there has been failure of the biological pacemaker to fire and/or bypass bridge to conduct current for more than a preset time interval. At this point the electronic pacemaker will take over the pacemaker function until the biological pacemaker resumes activity. Accordingly, a determination should be made as to when the electronic pacemaker will produce its pacemaker signal. State of the art pacemakers have the ability to detect when the heart rate falls below a threshold level in response to which an electronic pacemaker signal should be produced. The threshold level may be a fixed number, but preferably it varies depending on patient activity such as physical activity or emotional status. When the patient is at rest or pursuing light activity the patient's baseline heart rate may be at 60-80 beats per minute (bpm) (individualized for each patient), for example. This baseline heart rate varies depending on the age and physical condition of the patient, with athletic patients typically having lower baseline heart rates. The electronic pacemaker can be programmed to produce a pacemaker signal when the patient's actual heart rate (including that induced by any biological pacemaker) falls below a certain threshold baseline heart rate, a certain differential, or other ways known to those skilled in the art. When the patient is at rest the baseline heart rate will be the resting heart rate. The baseline heart rate will likely change depending on the physical activity level or emotional state of the patient. For example, if the baseline heart rate is 80 bpm, the electronic pacemaker may be set to produce a pacemaker signal when the actual heart rate is detected to be about 64 bpm (i.e., 80% of 80 bpm).

The electronic component can also be programmed to intervene at times of exercise if the biological component fails, by intervening at a higher heart rate and then gradually slowing to a baseline rate. For example, if the heart rate increases to 120 bpm due to physical activity or emotional state, the threshold may increase to 96 bpm (80% of 120 bpm). The biological portion of this therapy brings into play the autonomic responsiveness and range of heart rates that characterize biological pacemakers and the baseline rates that function as a safety-net, characterizing the electronic pacemaker. The electronic pacemaker may be arranged to output pacemaker signals whenever there is a pause of an interval of X % (e.g., 20%) greater than the previous interval, as long as the previous interval was not due to an electronic pacemaker signal and was of a rate greater than some minimum rate (e.g., 50 bpm).

Accordingly, in an embodiment of the present pacemaker systems, the electronic pacemaker senses the heart beating rate and produces a pacemaker signal when the heart beating rate falls below a specified level. In a further embodiment, the specified level is a specified proportion of the beating rate experienced by the heart in a reference time interval. In a still further embodiment, the reference time interval is an immediately preceding time period of specified duration.

As described herein, implanted biological pacemakers were tested in tandem with electronic pacemakers in canine studies. The electronic-demand pacemaker was set at a predesignated escape rate and the frequency of electronically versus biologically initiated heartbeats was monitored. In this way, the electronic component measures the efficacy of the biological component of a tandem pacemaker unit. It is expected that such tandem biological-electronic pacemakers will not only meet the patient protection standards required in Phase 1 and 2 clinical trials but will also offer therapeutic advantages over purely electronic pacemakers. That is, the biological component of the tandem system will function to vary heart rate over the range demanded by a patient's changing exercise and emotional status, while the electronic component will provide a safety net if the biological component were to fail either partially or totally. In addition, by reducing the frequency of electronic beats that would normally be delivered over time by an electronic-only pacemaker, the tandem unit will extend the battery life of the electronic component. This could profoundly increase the interval between which power packs require replacement. Hence, the components of the tandem pacemaker system operate synergistically in maximizing the opportunity for safe and physiologic cardiac rhythm control.

Methods of Treatment

The tandem pacemaker concept raises several issues with respect to clinical applications. First, the system is redundant by design and would have two completely unrelated failure modes. Two independent implant sites and independent energy sources would provide a safety mechanism in the event of a loss of capture (e.g., due to myocardial infarction). Second, the electronic pacemaker would provide not only a baseline safety net, but an ongoing log of all heartbeats for review by clinicians, thus providing insight into a patient's evolving physiology and the performance of their tandem pacemaker system. Third, since the biologic pacemaker will be designed to perform the majority of cardiac pacing, the longevity of the electronic pacemaker could be dramatically improved. Alternatively longevity could be maintained while the electronic pacemaker could be further reduced in size. Finally, the biological component of a tandem system would provide true autonomic responsiveness, a goal that has eluded more than 50 years of electronic pacemaker research and development.

The present invention also provides methods of treating various cardiac disorders by providing/administering a tandem system of the present invention to a subject. “Administering” shall mean delivering in a manner which is effected or performed using any of the various methods and delivery systems known to those skilled in the art. Administering can be performed, for example, pericardially, intracardially, subepicardially, transendocardially, via implant, via catheter, intracoronarily, endocardially, intravenously, intramuscularly, via thoracoscopy, subcutaneously, parenterally, topically, orally, intraperitoneally, intralymphatically, intralesionally, epidurally, or by in vivo electroporation. Administering can also be performed, for example, once, a plurality of times, and/or over one or more extended periods.

“Treating” a subject afflicted with a disorder shall mean causing the subject to experience a reduction, remission or regression of the disorder and/or its symptoms. In one embodiment, recurrence of the disorder and/or its symptoms is prevented. In a preferred embodiment, the subject is cured of the disorder and/or its symptoms.

“Inhibit” shall mean either lessening the likelihood of, or delaying, the disorder's onset, or preventing the onset of the disorder entirely. In a preferred embodiment, inhibiting the onset of a disorder means preventing its onset entirely.

A “subject” shall mean any animal or artificially modified animal. Animals include, but are not limited to, humans, non-human primates, dogs, cats, cows, horses, sheep, pigs, rabbits, ferrets, rodents such as mice, rats and guinea pigs, and birds such as chickens and turkeys. Artificially modified animals include, but are not limited to, SCID mice with human immune systems. In the preferred embodiment, the subject is a human.

The present invention also provides a method of treating a subject afflicted with a cardiac rhythm disorder, which method comprises administering to a subject a tandem pacemaker system of the present invention. A biological pacemaker is provided to the subject's heart to generate an effective biological pacemaker current. An electronic pacemaker is also provided to the subject's heart to work in tandem with the biological pacemaker to treat the cardiac rhythm disorder. The electronic pacemaker may be provided before, simultaneously with, or after the biological pacemaker. The electronic and the biological pacemaker are provided to the area of the heart best situated to compensate/treat the cardiac rhythm disorder. For example the biological pacemaker may be administered to, but not limited to, the Bachmann's bundle, sinoatrial node, atrioventricular junctional region, His branch, left or right atrial or ventricular muscle, left or right bundle branch, or Purkinje fibers of the subject's heart. The biological pacemaker is as described above and preferably enhances beta-adrenergic responsiveness of the heart, decreases outward potassium current I_{K1}, and/or increases inward current I_{f}.

The electronic pacemaker works in tandem with the biological pacemaker as described above. For example, the electronic pacemaker is programmed to sense the subject's heart beating rate and to produce a pacemaker signal when the heart beating rate falls below a selected heart beating rate. In other embodiments, the selected beating rate is a selected proportion of the beating rate experienced by the heart in a reference time interval. In other embodiments, the reference time interval is an immediately preceding time period of selected duration. As such, the battery life of the electronic pacemaker is preserved or lasts longer as it does not need to “fire” or send pacemaking signals as often since in the tandem system the biological pacemaker preferably generates an effective pacemaking signal. See FIGS. 29 and 30.

A cardiac rhythm disorder is any disorder that affects the heart beat rate and causes the heart rate to vary from a normal healthy heart rate. For example, the disorder may be, but is not limited to, a sinus node dysfunction, sinus bradycardia, marginal pacemaker activity, sick sinus syndrome, cardiac failure, tachyarrhythmia, sinus node reentry tachycardia, atrial tachycardia from an ectopic focus, atrial flutter, atrial fibrillation, or a bradyarrhythmia. In such situations, the biological pacemaker is preferably administered to the left or right atrial muscle, sinoatrial node or atrioventricular junctional region of the subject's heart.

This invention further provides a method of treating a subject afflicted with a cardiac rhythm disorder, wherein the disorder is a conduction block, complete atrioventricular block, incomplete atrioventricular block, bundle branch block, cardiac failure, or a bradyarrhythmia, comprising administering to the subject's heart any of the pacemaker systems described herein as comprising an atrioventricular bridge, such that the atrioventricular bridge spans the region exhibiting defective conductance, wherein propagation by the atrioventricular bridge of pacemaker activity induced by the electronic pacemaker is effective to treat the subject.

In certain embodiment of the present methods for treating cardiac rhythm disorders, a pre-existing source of pacemaker activity in the heart is ablated, so as not to conflict with the biological pacemaker and/or the electronic pacemaker.

In addition, the invention disclosed herein provides a method of treating a subject afflicted with a cardiac rhythm disorder comprising (a) providing a bypass bridge or in certain embodiments, an atrioventricular bridge in the heart, and (b) implanting an electronic pacemaker in the heart, so as to thereby treat the subject.

This invention further provides a method of inhibiting the onset of a cardiac rhythm disorder in a subject prone to such disorder comprising (a) inducing biological pacemaker activity in the subject's heart by functionally expressing in the heart at least one of (1) a nucleic acid encoding a HCN ion channel or a mutant or chimera thereof, (2) a nucleic acid encoding a MiRP1 beta subunit or a mutant thereof, and (3) a nucleic acid encoding both (i) a HCN ion channel or a mutant or chimera thereof and (ii) a MiRP1 beta subunit or a mutant thereof, at a level effective to induce a pacemaker activity in the heart; and (b) implanting an electronic pacemaker in the heart, so as to thereby inhibit the onset of the disorder in the subject. In certain embodiments, a biological pacemaker of the present invention is provided to a subject.

The present invention also provides a method of inducing in a cell a current capable of inducing biological pacemaker activity comprising administering to the heart any of the biological pacemakers described herein and thereby and functionally expressing in the heart a HCN ion channel or a mutant or chimera thereof, and/or a MiRP1 beta subunit or a mutant thereof, at a level effective to induce in the cell a current capable of inducing biological pacemaker activity, so as to thereby induce such current in the cell.

The invention disclosed herein also provides a method of increasing heart rate in a subject which comprises administering to the heart any of the biological pacemakers described herein and thereby expressing in the subject's heart a HCN ion channel or a mutant or chimera thereof, and/or a MiRP1 beta subunit or a mutant thereof, at a level effective to decrease the time constant of activation of the cell, so as to thereby increase heart rate in the subject.

The above-identified steps in the preceding method may also be used in methods of causing a contraction of a cell, shortening the time required to activate a cell, and changing the membrane potential of a cell.

Other Methods

The steps of the preceding method may also be used to preserve battery life of an electronic pacemaker implanted in a subject's heart, and to enhance the cardiac pacing function of an electronic pacemaker implanted in a subject's heart.

This invention further provides a method of monitoring cardiac signals with an electronic pacemaker having sensing capabilities implanted in a subject's heart comprising (a) selecting a site in or on the heart, (b) inducing biological pacemaker activity at the selected site by any of the methods described herein so as to enhance the natural pacemaker activity in the heart, (c) monitoring heart signals with the electronic pacemaker, and (d) storing the heart signals.

This invention also provides a method of enhancing the cardiac pacing function of an electronic pacemaker having sensing and demand pacing capabilities implanted in a subject's heart comprising (a) selecting a site in or on the heart, (b) inducing biological pacemaker activity at the selected site by any of the methods described herein so as to enhance the natural pacemaker activity in the heart, (c) monitoring heart signals with the electronic pacemaker, (d) determining when the heart should be paced based on the heart signals, and (e) selectively stimulating the heart with the electronic pacemaker when the natural pacemaker activity in tandem with the biological pacemaker activity fails to capture the heart.

Biventricular Pacing

A biological pacemaker, implanted at a site in a ventricle to optimize contraction, may also be used in a biventricular pacing mode in tandem with an electronic pacemaker. See Example 6. Thus, the invention provides a pacemaker system for treating a subject afflicted with ventricular dyssynchrony comprising (1) a biological pacemaker of the present invention to be administered to a site in one ventricle of the subject's heart, and (2) an electronic pacemaker to be administered to a site in the other ventricle of the subject's heart, wherein the electronic pacemaker is programmable to detect a signal from the biological pacemaker and to produce a pacemaker signal at a reference time interval after the biological pacemaker signal is detected, so as to thereby provide biventricular function. In one embodiment, the electronic pacemaker is also programmable to produce a pacemaker signal when it fails to detect a signal from the biological pacemaker after a time period of specified duration.

This invention also provides a pacemaker system for treating a subject afflicted with ventricular dyssynchrony comprising (1) a biological pacemaker of the present invention to be administered to a first ventricle of the subject's heart, (2) a first electronic pacemaker to be administered to a second ventricle of the subject's heart, and (3) a second electronic pacemaker to be administered to a coronary vein, wherein the second electronic pacemaker is programmable to detect a signal from the biological pacemaker and to produce a pacemaker signal in tandem with the first electronic pacemaker if said second electronic pacemaker fails to detect a signal from the biological pacemaker after a time period of specified duration, the first and second electronic pacemakers thereby providing biventricular function.

The present invention also provides a method of treating a subject afflicted with ventricular dyssynchrony comprising (a) selecting a site in a first ventricle of the subject's heart, (b) administering a biological pacemaker of the present invention at a selected site so as to induce pacemaker activity and stimulate contraction of the first ventricle, and (c) pacing a second ventricle of the heart with a first electronic pacemaker which is programmed to detect a signal from the biological pacemaker and to produce a pacemaker signal at a reference time interval after the biological pacemaker signal is detected, thereby providing biventricular function. Indifferent embodiments of this method, the biological pacemaker is introduced to the selected site via an endocardial approach, via the cardiac veins, or via thoracoscopy. In a preferred embodiment, biological pacemaker activity is induced on the lateral free wall with a bias towards the apex of the ventricle rather than the base. In another embodiment, the electronic pacemaker is also programmed to fire in an escape mode in the event the biological unit fires late, that is, the electronic pacemaker is programmed to produce a pacemaker signal when it fails to detect a signal from the biological pacemaker activity after a time period of specified duration.

In yet another embodiment, as an adjunct to the tandem system, a second electronic unit is placed in a coronary vein to function as a backup biventricular unit. In this arrangement, the second electronic pacemaker is programmed to detect a signal from the biological pacemaker and to produce a pacemaker signal in tandem with the first electronic pacemaker if it fails to detect a signal from the biological pacemaker after a time period of specified duration, the first and second electronic pacemakers thereby providing biventricular function.

Pharmaceutical Compositions Comprising Biological Pacemakers

This invention also provides a pharmaceutical composition comprising any one of the biological pacemakers, nucleic acids, recombinant vectors, cells, stem cells, HCN chimeric polypeptides or cardiomyocytes disclosed herein and a pharmaceutically acceptable carrier. Pharmaceutically acceptable carriers are well known to those skilled in the art and include, but are not limited to, 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Preservatives and other additives, such as, for example, antimicrobials, antioxidants and chelating agents may also be included with all the above carriers.

Polynucleotides, Polypeptides, Vectors and Cells

The present invention also provides a nucleic acid encoding a HCN ion channel or a mutant or chimera thereof, (2) a nucleic acid encoding a MiRP1 beta subunit or a mutant thereof, or (3) a nucleic acid encoding both (i) a HCN ion channel or a mutant or chimera thereof and (ii) a MiRP1 beta subunit or a mutant thereof, as described above, as well as the polypeptides per se. The invention also provides a nucleic acid encoding a HCN channel that has at least about 75% sequence identity with mHCN1 (SEQ ID NO: ______), mHCN2 (SEQ ID NO:______), mHCN3 (SEQ ID NO:______), or mHCN4 (SEQ ID NO:______), as well as the polypeptides per se and which are capable of inducing a biological pacemaker current and preferably have improved characteristics as compared to wild type HCN channels, such as faster kinetics, more positive activation, increased expression levels, increased stability, enhanced cAMP responsiveness, and enhanced neurohumoral response.

The invention also provides a recombinant vector comprising an expression vector and inserted therein any of the nucleic acids disclosed in this application (i.e. HCN channels, mutant HCN channels, chimeric HCN channels and MiRP1. A “vector” shall mean any nucleic acid vector known in the art. Such vectors include, but are not limited to, plasmid vectors, cosmid vectors and viral vectors. Several eukaryotic expression plasmids, including pCI, pCMS-EGFP, pHygEGFP, pEGFP-C1, and shuttle plasmids for Cre-lox Ad vector construction, pDC515 and pDC516, are used in constructs described herein. However, the invention is not limited to these plasmid vectors or their derivatives, and may include other vectors known to those skilled in the art. Thus, the invention provides a recombinant vector comprising an expression vector and inserted therein (1) a nucleic acid encoding a HCN ion channel or a mutant or chimera thereof, (2) a nucleic acid encoding a MiRP1 beta subunit or a mutant thereof, or (3) a nucleic acid encoding both (i) a HCN ion channel or a mutant or chimera thereof and (ii) a MiRP1 beta subunit or a mutant thereof. In various embodiments, the expression vector is a viral vector, a plasmid vector, or a cosmid vector. In further embodiments, the viral vector is an adenoviral, AAV, or retroviral vector.

This invention also provides a cell comprising any of the recombinant vectors described herein, wherein the cell expresses the nucleic acid inserted in the expression vector. This cell is as described above with respect to cells useful in biological pacemakers.

The following Examples are presented to aid in understanding the invention, and are not intended, and should not be construed, to limit in any way the invention set forth in the claims which follow thereafter. These Examples do not include detailed descriptions of experimental methods that are well known to those of ordinary skill in the art, such as methods used in the construction of recombinant nucleic acid vectors, transfection of host cells with such recombinant vectors, and the functional expression of genes in transfected cells. Detailed descriptions of such conventional methods are provided in numerous publications, including Sambrook et al. (1989), the contents of which are hereby incorporated herein in their entirety.

### EXAMPLE 1

### Expression and Electrophysiological Characterization of HCN Channels in Cultured Cells

Isolation and Culture of Cardiomyocytes and Xenopus laevis Oocytes

Adult rats were anesthetized with ketamine-xylazine before cardiectomy, and neonatal rats decapitated. Newborn rat ventricular myocyte cultures were prepared as previously described (Protas and Robinson, 1999). Briefly, 1-2-day-old Wistar rats were euthanized, hearts were quickly removed and ventricles were dissociated using a standard trypsinization procedure. Myocytes were harvested, preplated to reduce fibroblast proliferation, cultured initially in serum-containing medium (except when being transfected with plasmids as described below), and then incubated in serum free medium (SFM) at 37° C., 5% CO₂ after 24 h. Action potential studies were conducted on 4-day-old monolayer cultures plated directly onto fibronectin-coated 9×22 mm glass coverslips. For voltage clamp experiments, 4-6 day old monolayer cultures were resuspended by brief (2-3 min) exposure to 0.25% trypsin, then replated onto fibronectin-coated coverslips and studied within 2-8 h.

Freshly isolated adult ventricular myocytes were prepared using the procedure described by Kuznetsov et al. (1995). This entailed a Langendorff perfusion of collagenase, followed by trimming away of the atria. The remaining tissue was minced and dissociated in additional collagenase solution. The isolated myocytes were suspended in a SFM then plated on 9×22 mm glass coverslips at 0.5-1×10³ cells/mm². Two to three hours later, after the myocytes had adhered to the coverslips, the adenoviral infection procedure was begun (see below).

For preparation of canine myocytes, adult dogs of either sex were killed using an approved protocol by an injection of sodium pentobarbital (80 mg kg⁻¹ body weight). Cardiomyocytes were isolated from the canine ventricle as previously described (Yu et al., 2000). A method of primary culture of canine cardiomyocytes was adapted from the procedure described for mouse cardiomyocytes (Zhou et al., 2000). The cardiomyocytes were plated at 0.5-1 (10⁴ cells cm⁻² in minimal essential medium (MEM) containing 2.5% fetal bovine serum (FBS) and 1% penicillin/streptomycin (PS) onto mouse laminin (10 μg ml⁻¹) precoated coverslips. After 1 h of culture in a 5% CO₂ incubator at 37° C., the medium was changed to FBS-free MEM. Stem cells were added after 24 h and coculture was maintained in Dulbecco's modified Eagle's medium (DMEM) with 5% FBS. Cell Tracker Green (Molecular Probes, Eugene, Oreg.) was used to distinguish hMSCs from HeLa cells in coculture in all experiments (Valiunas et al., 2000).

Oocytes were prepared from mature female Xenopus laevis in accordance with an approved protocol as previously described (Yu et al., 2004).

Expression of Wild-Type and Mutant HCN Channels in Cardiomyocytes and Oocytes

cDNAs encoding mouse HCN2 (mHCN2, GenBank AJ225122) or HCN4 (mHCN4, GenBank deposit in progress) were subcloned into the pCI mammalian expression vector (Promega, Madison, Wis.). The resulting plasmids (pCI-mHCN2 or pCI-mHCN4) were used for neonatal rat ventricular myocyte transfection, as indicated. A separate plasmid (pEGFP-CI; Clontech, Palo Alto, Calif.) expressing the gene of enhanced green fluorescent protein (EGFP) as a visual marker for successful DNA transfer was included in all transfection experiments. For transfection, 2 μg of pCI-mHCN and 1 pg of pEGFP-CI were first incubated in 200 μl of SFM containing 10 μl of lipofectin (Gibco Life Technologies, Rockville, Md.) at room temperature for 45 min. The mixture was then added to a 35-mm petri dish containing 106 cells suspended in 0.8 ml of SFM. After overnight incubation at 37° C. in a CO₂ incubator, the medium containing the plasmids and lipofectin was discarded and the dish was refilled with 2 ml of fresh SFM. Patch clamp experiments were carried out on resuspended cells exhibiting detectable levels of GFP by fluorescence microscopy 3-5 days after transfection.

For increased expression efficiency, an adenoviral construct for mHCN2 was prepared. Gene delivery and transfer procedures followed previously published methods (Ng et al., 2000; He et al., 1998). A DNA fragment (between EcoRI and XbaI restriction sites) that included mHCN2 DNA downstream of the CMV promoter was obtained from plasmid pTR-mHCN2 (Santoro and Tibbs, 1999) and subcloned into the shuttle vector pDC516 (AdMax™; Microbix Biosystems, Toronto, Canada). The resulting pDC516-mHCN2 shuttle plasmid was co-transfected with a 35.5 kb El-deleted Ad genomic plasmid pBHGΔE1,3FLP (AdMax™) into El-complementing HEK293 cells. Successful recombination of the two vectors resulted in production of the adenovirus mHCN2 (AdmHCN2), which was subsequently plaque-purified, amplified in HEK293 cells, and harvested after CsCl-banding to achieve a titer of at least 10¹¹ ffu/ml.

An adenoviral construct of mouse mHCN2 (AdmHCN2) was also prepared as previously described (Qu et al., 2001). The mE324A point mutation was introduced into the mHCN2 sequence with the QuikChange® XL Site-Directed Mutagenesis Kit (Stratagene, La Jolla, Calif.) and packaged in the pDC515 shuttle vector (AdMax™, Microbix Biosystems) to create pDC515mE324A. PDC515mE324A then was co-transfected with pBHGfrtΔE1,3FLP into El-complimenting HEK293 cells. The adenoviral construct AdmE324A was subsequently harvested and CsCl purified. For consistency with earlier studies (Qu et al., 2003), when preparing samples for in vivo injection, 3×10¹⁰ ffu of each adenovirus was mixed with an equal amount of a GFP-expressing adenovirus (AdGFP) in a total volume of 700 μl.

AdHCN2 infection of rat ventricular myocytes was carried out 2-3 h after the isolated cells were plated onto coverslips. The culture medium was removed from the dishes (35-mm) and the inoculum of 0.2-0.3 ml/dish was added containing AdHCN2. The value of m.o.i. (multiplicity of infection—the ratio of viral units to cells) was 15-100. The inoculum was dispersed over the cells every 20 min by gently tilting the dishes so that the cells were evenly exposed to the viral particles. The dishes were kept at 37° C. in a CO₂ incubator during the adsorption period of 2 h, then the inoculum was discarded and the dishes were washed and refilled with the appropriate culture medium. The dishes remained in the incubator for 24-48 h before electrophysiological experiments were conducted.

Adenoviral infection of the newborn ventricular myocytes was performed on cell monolayer cultures 4 days after initial plating. Cells were exposed to a virus-containing mix (m.o.i. 20, in 250 μl of culture medium) for 2 h, rinsed twice and incubated in SFM at 37° C., 5% CO₂ for 24-48 hours prior to the cells being resuspended as described above for electrophysiological study. In early experiments, AdGFP was employed but since >90% of cells exposed to AdmHCN2 in vitro were found to express the current (Qu et al., 2001), in later experiments cells were not co-infected with AdGFP to aid in the selection of infected cells.

For expression of HCN in Xenopus oocytes, oocytes were injected with 5 ng of cRNA made from mouse wild-type mHCN2 and mutant mHCN2 (E324A) plasmids. Injected oocytes were incubated at 18° C. for 24-48 h prior to electrophysiological analysis.

Electrophysiological Measurements in Cultured Cardiomyocytes and Oocytes

Voltage and current signals were recorded using patch clamp amplifiers (Axopatch 200). The current signals were digitized with a 16 bit A/D-converter (Digidata 1322A, Axon Instruments, Union City, Calif.) and stored with a personal computer. Data acquisition and analysis were performed with pCLAMP 8 software (Axon Instruments). Curve fitting and statistical analyses were performed using SigmaPlot and SigmaStat, respectively (SPSS, Chicago, Ill.).

The whole-cell patch clamp technique was employed to record mHCN2 current from cultured myocytes. Experiments were carried out on cells superfused at 35° C. The external solution contained (mM): NaCl, 140; NaOH, 2.3; MgCl₂, 1; KCl, 10; CaCl₂, 1; HEPES, 5; glucose, 10; pH 7.4. MnCl₂ (2 mM) and BaCl₂ (4 mM) were added to block other currents. The pipette solution contained (mM): aspartic acid, 130; KOH, 146; NaCl, 10; CaCl₂, 2; EGTA-KOH, 5; Mg-ATP, 2; HEPES-KOH, 10; pH 7.2.

To measure the HCN activation curve, a standard two-step protocol was employed. Hyperpolarizing steps from −25 to −135 mV for mHCN2 and from −5 or −15 to −135 mV for mE324A were applied from a holding potential of −10 mV, followed by a tail current step (to −125 or −135 mV). The duration of test steps was longer at less hyperpolarized potential for mHCN2 channels, to more closely approach steady-state activation at all voltages. The normalized plot of tail current versus test voltage was fit with a Boltzmann function and then the voltage of half maximum activation (V_{1/2}) and slope factor(s) were defined from the fitting. Activation kinetics were determined from the same traces, while deactivation kinetics were determined from traces recorded at each test potential after achieving full activation by a prepulse to −135 mV. Time constants were then obtained by fitting the early time course of activation or deactivation current traces with a monoexponential function; the initial delay and any late slow activation or deactivation phase were ignored (Qu et al., 2001; Altomare et al., 2001). Current densities are expressed as the value of the time-dependent component of current amplitude, measured at the end of the test potential and normalized to cell membrane capacitance. Records were not corrected for liquid junction potential, which was previously determined to be 9.8 mV under these conditions (Qu et al., 2001).

For measurements in Xenopus oocytes, oocytes were voltage-clamped using a two-microelectrode voltage clamp technique. The extracellular recording solution (OR2) contained (in mM): NaCl, 80; KCl, 2; MgCl₂, 1; and Na-HEPES, 5 (pH 7.6). For the recording of steady state activation of expressed Wt mHCN2, currents were elicited by 2-s long hyperpolarizing pulses between −30 mV and −160 mV with 10 mV increments, followed by a 1-s depolarizing pulse to +15 mV. The holding potential was −30 mV. As to the mHCN2 (E324A), currents were elicited by 3-s-long hyperpolarizing pulses between +20 mV and −130 mV with 10 mV increments, followed by a 1 second depolarizing pulse to +50 mV. The holding potential was +20 mV. To construct the current/voltage relationship for wildtype (Wt) mHCN2, the cell was held at −30 mV, the current was elicited by a 2-s hyperpolarizing voltage step to −140 mV to saturate activation, and followed by 2-s depolarizing voltage steps between −80 mV and +50 mV in 10 mV increments. For mHCN2 (E324A), the cell was held at +20 mV, current was elicited by a 1.5-s hyperpolarizing voltage step to −110 mV to saturate activation, and then followed by 1.5-s depolarizing voltage steps between −80 mV and +50 mV in 10 mV increments for the recording of tail currents. To record the current amplitudes for Wt mHCN2, the current was evoked by applying a 3-s hyperpolarizing voltage pulse to −120 mV from a holding potential of −30 mV. For mHCN2 (E324A), the current was evoked by applying a 3-s hyperpolarizing voltage pulse to −120 mV from a holding potential of +20 mV.

Data are presented as means±SEM. Experimental data were compared using a Student's t-test or Chi-square test with Yates' correction, as appropriate. When making comparisons, matched cells from the same cultures were used, and data from at least 3 separate cultures were pooled for each comparison.

Pacemaker Currents Induced by mHCN2 and mE324a in Cardiomyocytes

Previous experiments have shown that overexpression of HCN2 in neonatal rat myocytes in culture induced a pacemaker current which increased beating rate, and that mutations in the HCN2 pacemaker gene and/or the addition of appropriate accessory channel subunits altered the characteristics of the expressed current in ways that would be expected to further enhance the beating rate (U.S. Pat. No. 6,849,611; Qu et al., 2001; Qu et al., 2004). Infection with an Ad expressing HCN2 also significantly increased the spontaneous beating rate of monolayer cultures of synchronously beating (U.S. Pat. No. 6,849,611; Qu et al. 2001). Myocyte cultures were also infected with the HCN2 adenovirus and a second virus carrying either GFP or an HA-tagged form of MiRP1 which is the beta subunit for HCN2. The result was a significant increase in current magnitude and acceleration of activation and deactivation kinetics (Qu et al., 2004).

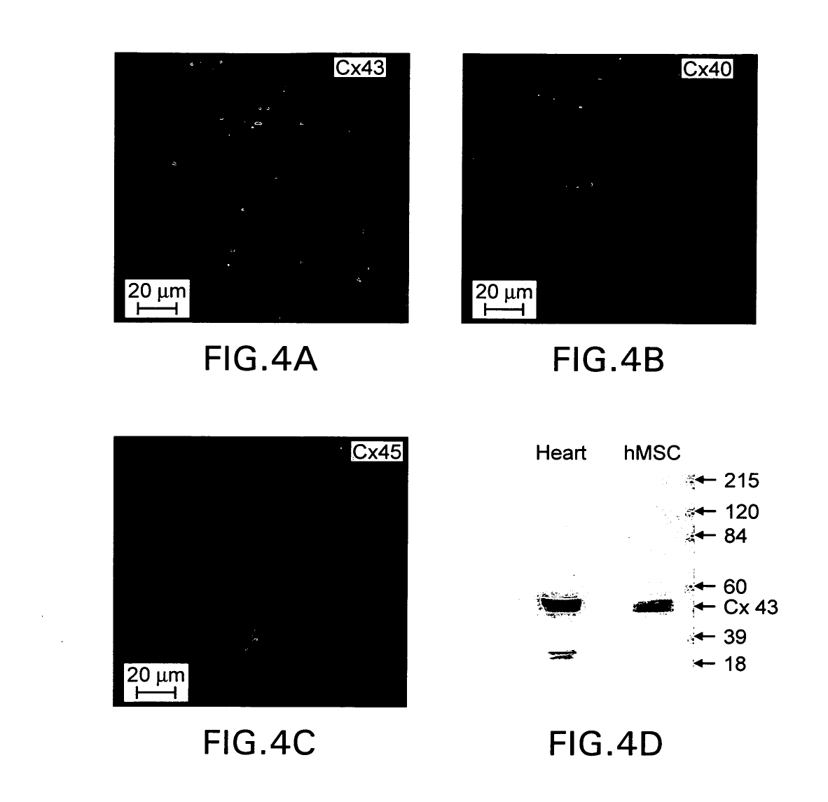

In the whole-cell voltage clamp experiments described herein, mHCN2- and mE324A-expressing myocytes both gave rise to an inward current in response to hyperpolarizing voltages. Representative normalized current traces obtained at test potentials ranging from −25 to −125 mV, from a holding potential of −10 mV, are shown in FIGS. 4A and B. It is apparent from the expanded currents in the insets that the activation threshold of mE324A channels is less negative than that of mHCN2 channels.

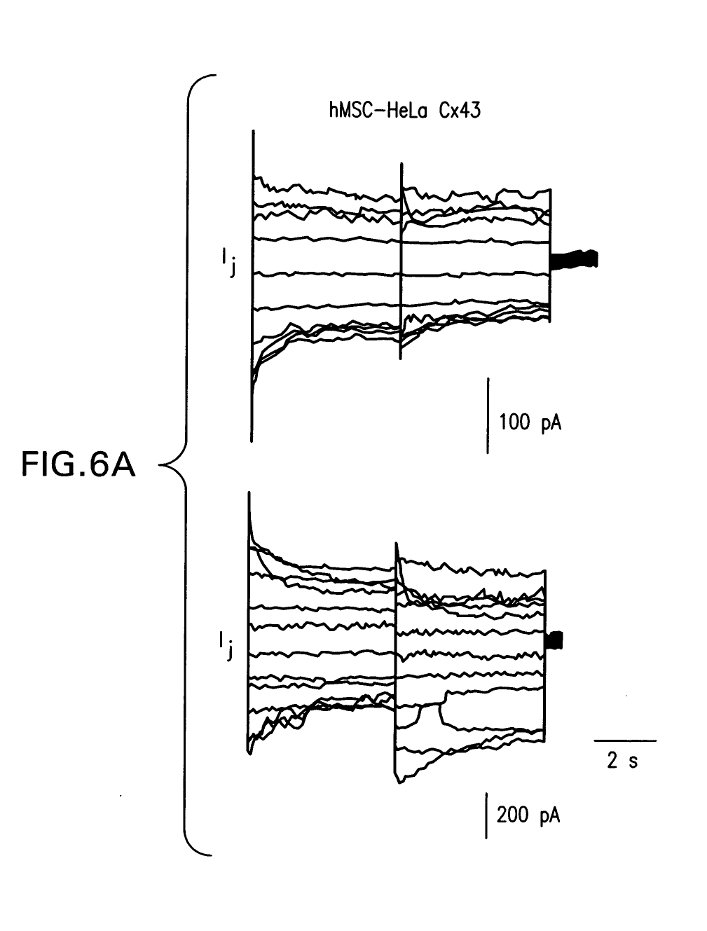

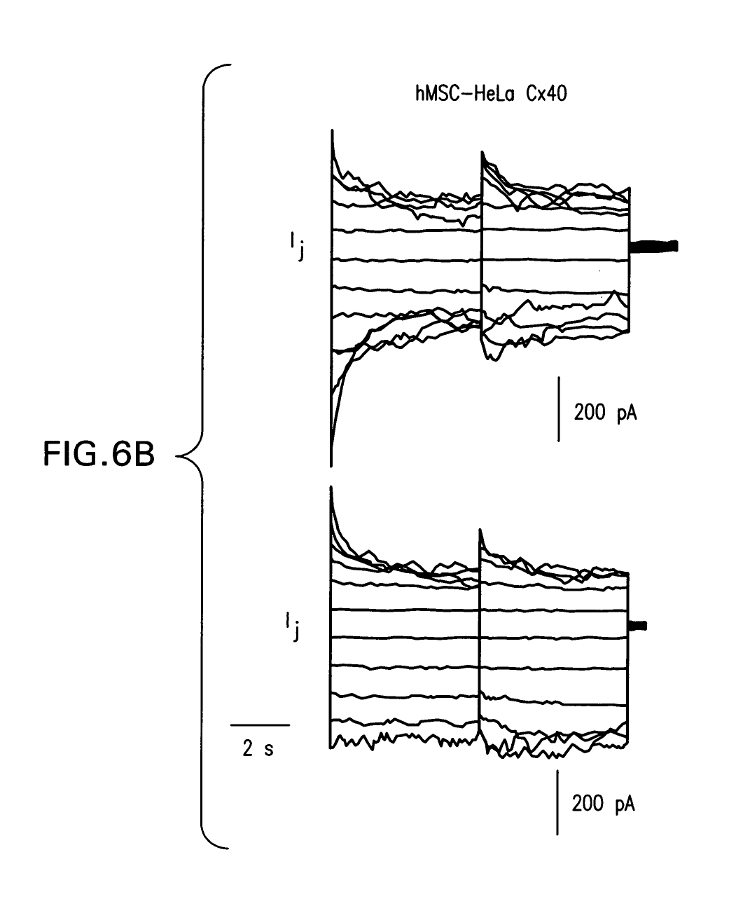

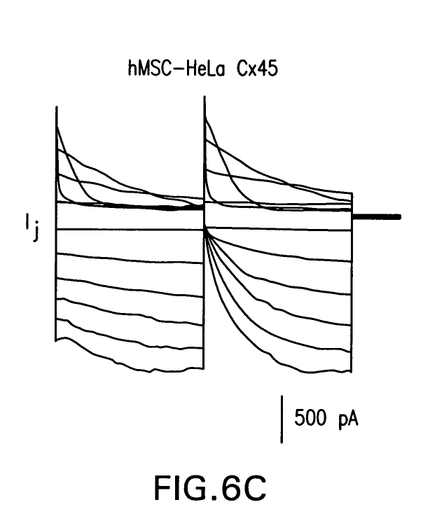

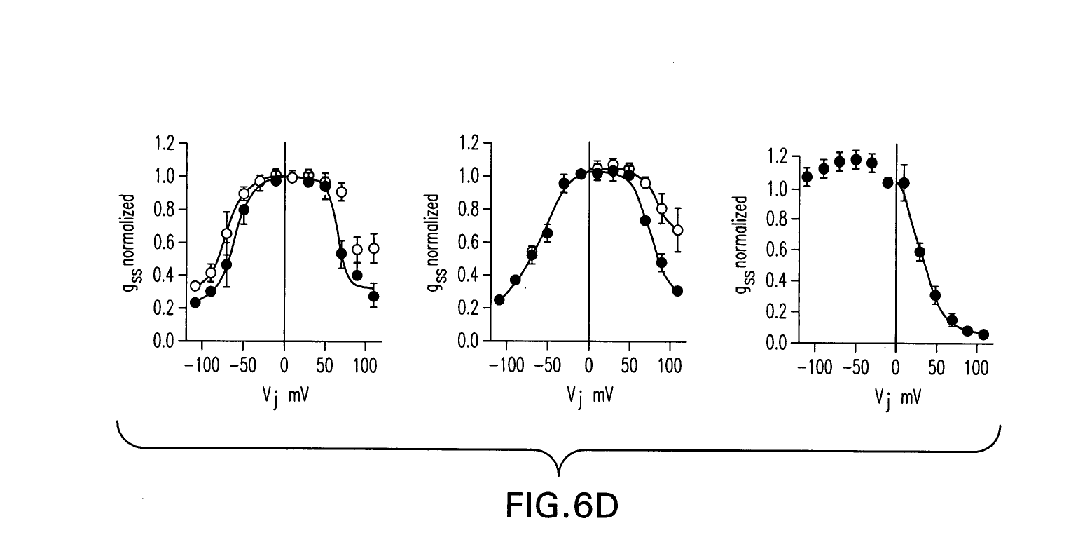

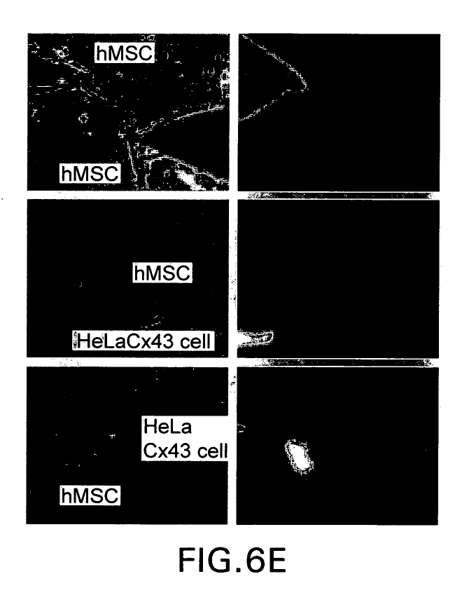

The difference in voltage dependence of activation between mHCN2 and mE324A is more evident from the mean current-voltage relationships shown in FIG. 4C. The curves were obtained from tail currents, as described above. The individual activation curves were each fit to the Boltzmann equation and the calculated midpoint (V_{1/2}) and slope factor (s) from all cells averaged and statistically compared. Mean parameters for mHCN2 (n=14) and mE324A (n=16) expressing cells, respectively, were: V_{1/2}=−66.1±1.5 mV and −46.9±1.2 mV (P<0.05) and s=10.7±0.5 mV and 9.6±0.4 mV (p>0.05). Thus, in agreement with data previously obtained in oocytes (Chen et al., 2001) and confirmed herein (see FIGS. 6-9), the mE324A mutation resulted in a positive shift of the activation curve relative to that of mHCN2 when both constructs were expressed in newborn myocytes.

The activation kinetics of mE324A channels appeared faster than those of mHCN2 (FIGS. 4A, B insets). To demonstrate this difference, time constants of activation and deactivation were measured at different voltages as described above, and averaged (FIG. 4D). These data show that the faster activation kinetics observed for mE324A channels were due to a positive shift of the voltage-dependence of gating kinetics. Both activation and deactivation voltage dependence shifted positively, so that at the positive voltages at which deactivation was measured, the deactivation was slower for mE324A than for mHCN2. Moreover, this shift is comparable to that in the current-voltage relationship. Indeed the relative peaks of the kinetic-voltage relations were consistent with the previously determined V_{1/2} values.

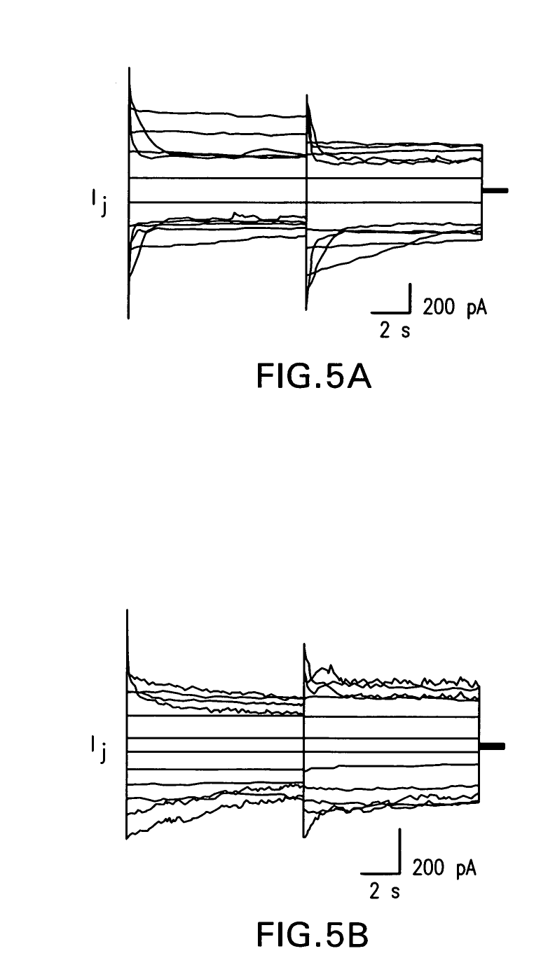

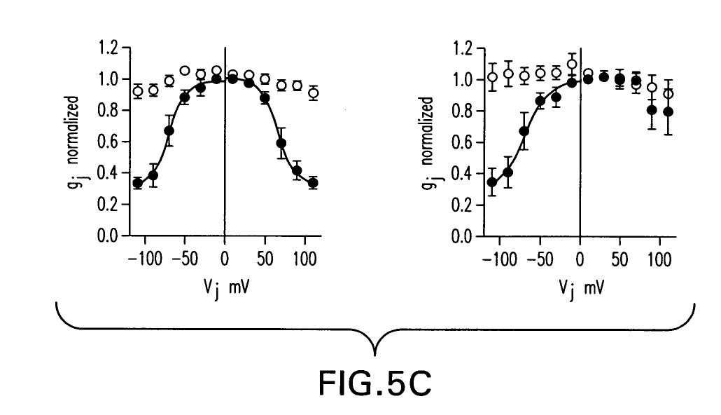

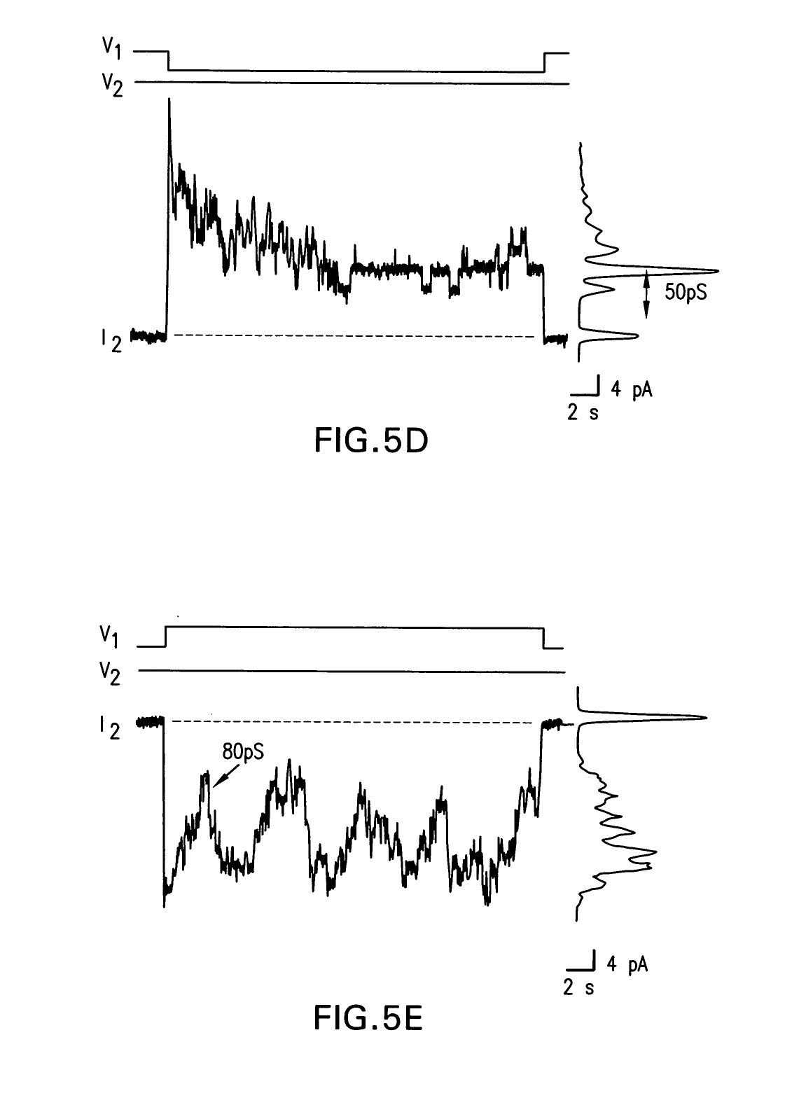

The positive shift in the activation relation and kinetics would be expected to result in more current being passed earlier in the cardiac cycle with mE324A in comparison to mHCN2. However, to be beneficial as a biological pacemaker, it also is necessary to preserve autonomic responsiveness. To assess this, mHCN2 and mE324A activation curves were compared in the absence and in the presence of cAMP in the pipette solution (FIG. 5). Both channels responded to the presence of saturating intracellular cAMP as detailed in the brief description of FIG. 5.

Whether the mutant channel expressed current as well as the wild-type was also investigated. The percentage of myocytes expressing mE324A current was significantly smaller than the percentage expressing mMCN2 (36.6% of 93 cells vs. 74.5% of 47 cells respectively, P<0.05) in 6 matched cell cultures. Moreover, in the cells that did express current the mE324A current density (measured at −135 mV) was about 2.5 times smaller than that of mHCN2 (21.0±3.5 pA/pF, n=12, vs. 53.5±8.7 pA/pF, n=10, respectively, P<0.05).

Currents Induced by mHCN2 and mE324A in Xenopus Oocytes

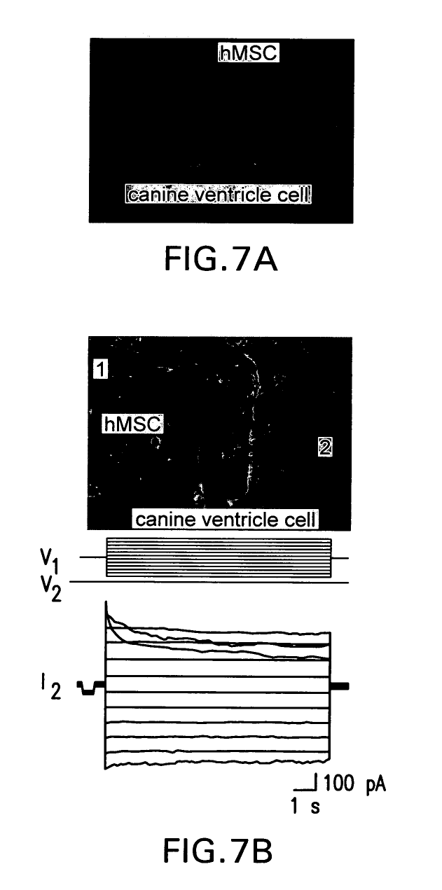

FIG. 6 shows activation properties and kinetics of the heterologously expressed current. In oocytes, the mHCN2 activates 35 mV more negatively than mE324A. This more positive activation is accompanied by both a shift in the voltage dependence of the kinetics of activation as well as more rapid kinetics at the midpoint of activation for mE324A. Both mHCN2 and mE324A responded to application of 8-Br-cAMP (1 mM) with a positive shift in activation (FIG. 7). For mHCN2, cAMP shifted the Vₕ by approximately 8 mV (Vₕ values were −92.7 mV±1.1 mV for control and −84.9 mV±0.7 mV for cAMP (n=6, P<0.01), and the corresponding slope (s) values were 13.9 mV±1.0 mV and 9.5 mV±0.6 mV (n=6, p>0.05). For mE324A, cAMP positively shifted the Vₕ by approximately 7 mV (Vₕ values were −57.3 mV±1.6 mV for control and −48.9 mV±1.8 mV for cAMP (n=9, P<0.01), and the corresponding slope (s) values were 15.2 mV±1.3 mV and 19.7 mV±0.1 mV (n=9, p>0.05).

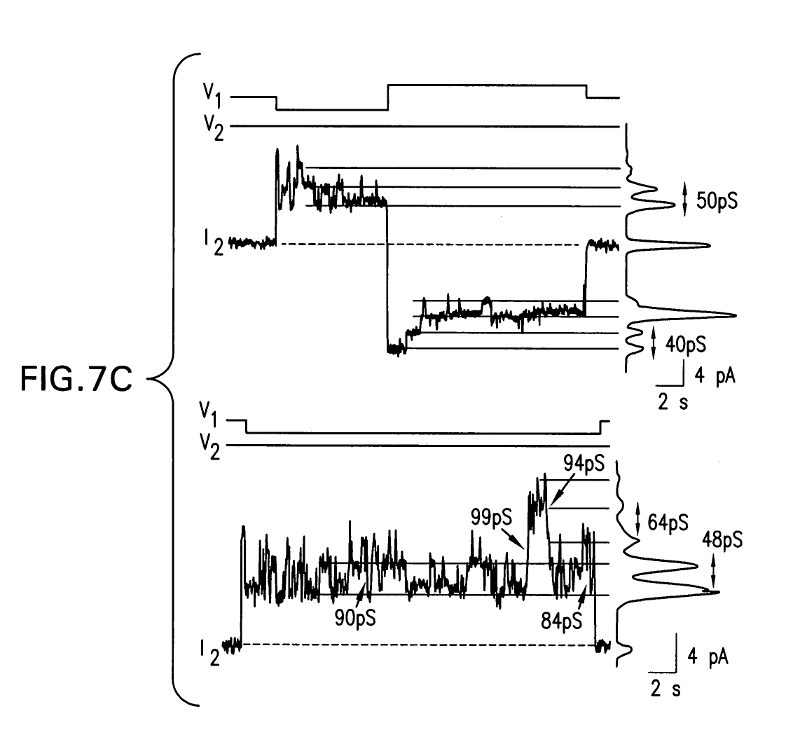

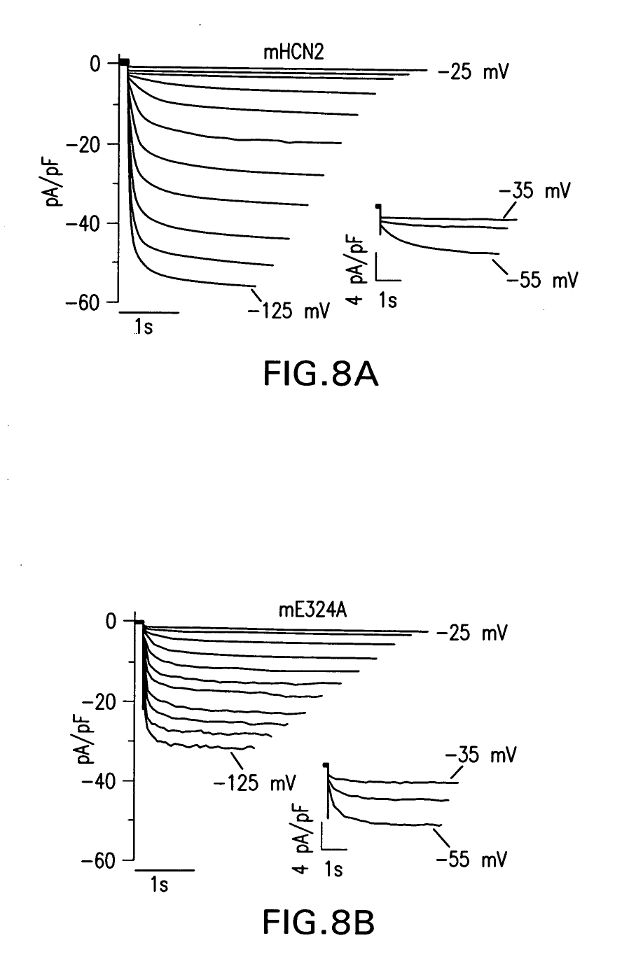

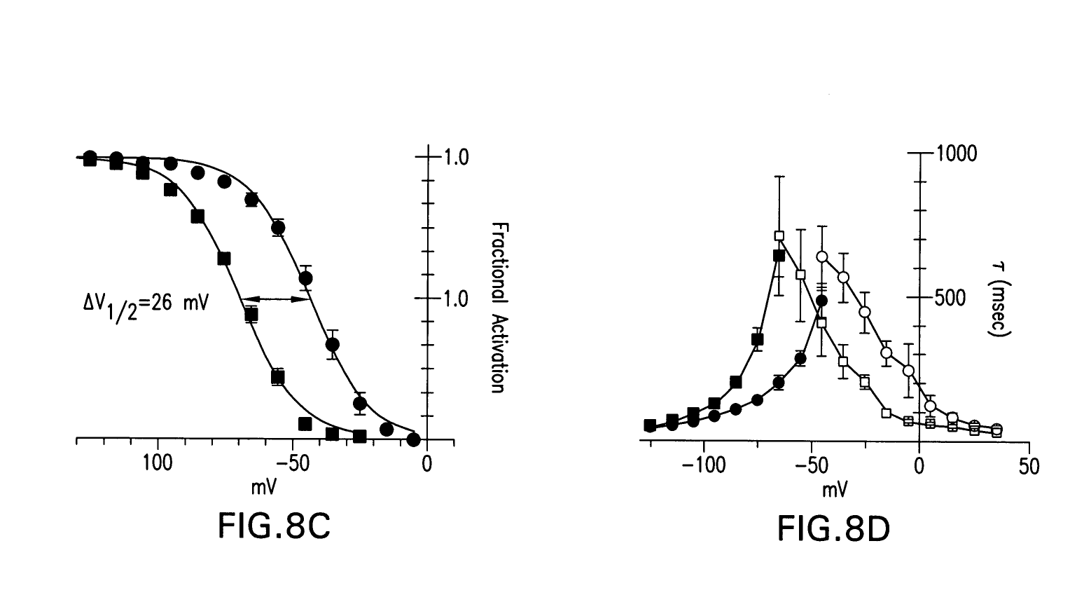

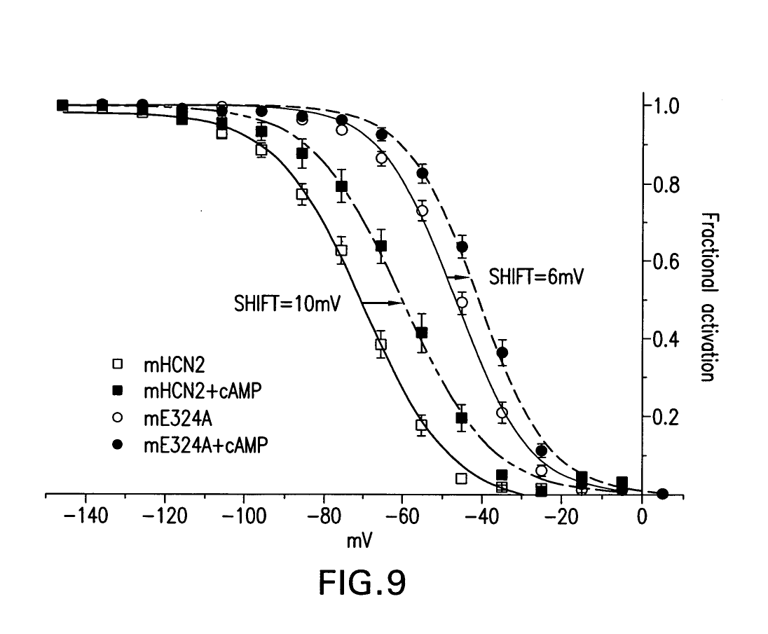

Both mHCN2 and mE324A generated inward currents blocked by 5 mM Cs⁺ with reversal potentials near −40 mV (FIG. 8). Finally, a single voltage pulse was applied near saturation (−120 mV) to compare the levels of expression of mHCN2 and mE324A. The HCN2 induced current was 912.7±63.7 nA, n=9, while the E324A induced current was 579.8±18.2 nA, n=9 (P<0.01). Thus, there was significantly reduced expression for those oocytes expressing mE324A (see FIG. 9).

### EXAMPLE 2

### Induction of Pacemaker Activity by Overexpression of HCN Channels in Heart in Situ

HCN2 Induces Pacemaker Current in Heart In Situ

It was hypothesized that overexpression of I_{f} in either secondary pacemaker tissues of the cardiac specialized conducting system or in non-pacemaker cells of the myocardium could provide a nidus of pacemaker activity to drive the heart in a “demand” mode in the absence of dominant pacemaker function of the sinus node or failure of impulse propagation via the atrioventricular node. Attention was focused on HCN2 because its kinetics are more favorable than those of HCN4 and its cAMP responsiveness is greater than that of HCN1. Initial experiments were performed in neonatal rat myocytes in culture. These experiments indicated that not only could an overexpressed pacemaker current increase beating rate, but that mutations in the HCN2 pacemaker gene and/or the addition of appropriate accessory channel subunits could modify the characteristics of the expressed current in a manner that might be expected to further enhance the beating rate (U.S. Pat. No. 6,849,611; Qu et al., 2001; Qu et al., 2004; Chen et al., 2001b; Plotnikov et al., 2005a). These neonatal ventricular myocytes manifest a small endogenous pacemaker current and, when infected with an adenovirus carrying HCN2, express a markedly larger pacemaker current. When the spontaneous beating rate of monolayer cultures infected with an Ad expressing HCN2 and the green fluorescent protein (GFP) was compared with a virus incorporating GFP as a control and marker, the HCN2/GFP-expressing cultures beat significantly faster (Qu et al. 2001).

Based on the encouraging results and implications of the cell culture work, proof of concept was tested by injecting a small quantity of HCN2 and GFP genes in an adenoviral vector into canine left atrium (Qu et al., 2003). After recovery of the animals, the right vagus nerve was stimulated to induce sinoatrial slowing and/or block. In this setting, pacemaker activity originated in the left atrium and was pace-mapped to the site of adenoviral injection. Increasing the intensity of the vagal stimulation and adding left vagal stimulation as well caused cessation of biological pacemaker activity, implying parasympathetic responsiveness. The atrial myocytes were disaggregated from the site of injection, and overexpressed pacemaker current was demonstrated. In sum, the results indicate that such overexpressed pacemaker current could provide escape beats under circumstances of sinus slowing (Qu et al. 2003).

The next steps involved catheter injection of the same adenoviral HCN2/GFP construct into the canine proximal LV conducting system, under fluoroscopic control (Plotnikov et al., 2004). Animals so injected demonstrated idioventricular rhythms having rates of 50-60 bpm when sinus rhythm was suppressed by vagal stimulation. For the HCN2 group, the rhythms mapped to the site of injection. When bundle branch tissues were removed from the heart and studied with microelectrodes, automaticity in those injected with HCN2 was found to exceed that in control preparations, i.e., there was a significantly greater spontaneous rate generated by the HCN2 injected bundle branches than by those injected with either saline or virus carrying GFP alone (Plotnikov et al., 2004).

Biophysical Properties of Ion Currents as Predictors of Biological Pacemaker Function

The studies in neonatal rat myocytes (FIGS. 4 and 5) and in Xenopus oocytes (FIGS. 6-9) gave concordant results with regard to the function of mHCN2 and mE324A. That the mE324A mutation induced faster, more positive pacemaker current activation in these in vitro settings than did mHCN2 might be interpreted as suggesting the mutant channel would result in a faster pacemaker rate and/or a shorter escape interval after overdrive pacing than occurred in saline-injected or mHCN2 injected hearts. However, in situ both the saline- and mHCN2-injected hearts showed escape times equivalent to the mE324A-injected hearts. As for automatic rates per se, these were equivalent for mHCN2— and mE324A-injected hearts, and both were significantly faster than those injected with saline. In other words, for two important descriptors, rate attained and overdrive suppression, there was no clear discrimination between the effects of mHCN2 and mE324A in situ.

One explanation for this may be that the percent of myocytes expressing mE324A current was significantly less than that expressing mHCN2. Moreover there was a lesser current density in the E324A group. Thus, while a greater fraction of channels activate faster at a given voltage with mE324A compared to mHCN2, the total number of channels available or net current may be approximately equivalent at physiologically relevant voltages such as −55 mV (see insets in FIG. 4).

The extent to which biophysical results were predictive of those in situ is seen in the following: the biophysical data indicating that mE324A density is less than that of mHCN2, and that mE324A activation is positive to and faster than that of mHCN2, would suggest that for pacemaker rate there may be no advantage to either construct. The finding that the mE324A cAMP response is positive to that of mHCN2 would suggest that the magnitude or sensitivity of the mE324A response to epinephrine in situ might be greater than that for mHCN2. In fact, the studies in situ showed no rate advantage to either construct with a greater response to epinephrine of the mE324A mutant. Not only does this show concordance between biophysical finding and clinical implication, but it leads to the following hypotheses: first, as long as there is sufficient current density, a positive position of the activation curve and/or faster kinetics are more important than absolute current density in biological pacemaker functionality; and second, adrenergic responsiveness depends on the final position of the activation curve in the presence of cAMP more than the magnitude of the voltage shift.

### EXAMPLE 3

### Cell Therapy with Human Mesenchymal Stem Cells

Cell Cultures

Human mesenchymal stem cells (hMSCs; mesenchymal stem cells, human bone marrow; Poietics™) were purchased from Clonetics/BioWhittaker (Walkersville, Md., USA), cultured in mesenchymal stem cell (MCS) growth medium and used from passages 2-4. Isolated and purified hMSCs can be cultured for many passages (12) without losing their unique properties, i.e., normal karyotype and telomerase activity (van den Bos et al., 1997; Pittenger et al., 1999).

HeLa cells transfected with rat Cx40, rat Cx43 or mouse Cx45 were cocultured with hMSCs. Production, characterization and culture conditions of transfected HeLa cells have been previously described (Valiunas et al., 2000; 2002).

Anti-Connexin Antibodies, Immunofluorescent Labeling, and Immunoblot Analysis

Commercially available mouse anticonnexin monoclonal and polyclonal antibodies (Chemicon International, Temecula, Calif.) of Cx40, Cx43 and Cx45 were used for immunostaining and immunoblots as described earlier (Laing and Beyer, 1995). Fluorescein-conjugated goat antimouse or antirabbit IgG (ICN Biomedicals, Inc., Costa Mesa, Calif.) was used as secondary antibody.

Electrophysiological Measurements Across Gap Junctions

Glass coverslips with adherent cells were transferred to an experimental chamber perfused at room temperature (˜22° C.) with bath solution containing (mM): NaCl, 150; KCl, 10; CaCl₂, 2; Hepes, 5 (pH 7.4); glucose, 5. The patch pipettes were filled with solution containing (mM): potassium aspartate, 120; NaCl, 10; MgATP, 3; Hepes, 5 (pH 7.2); EGTA, 10 (pCa ˜8); filtered through 0.22 μm pores. When filled, the resistance of the pipettes measured 1-2 MΩ. Experiments were carried out on cell pairs using a double voltage-clamp. This method permitted control of the membrane potential (Vₘ) and measurement of the associated junctional currents (Iⱼ).

Dye Flux Studies

Dye transfer through gap junction channels was investigated using cell pairs. Lucifer Yellow (LY; Molecular Probes) was dissolved in the pipette solution to reach a concentration of 2 mM. Fluorescent dye cell-to-cell spread was imaged using a 16 bit 64000 pixel grey scale digital CCD-camera (LYNXX 2000T, SpectraSource Instruments, Westlake Village, Calif.) (Valiunas et al., 2002). In experiments with heterologous pairs, LY was always injected into the cells which were tagged with Cell Tracker Green. The injected cell fluorescence intensity derived from LY is 10-15 times higher than the initial fluorescence from Cell Tracker Green.

Human MSCs Express Connexins

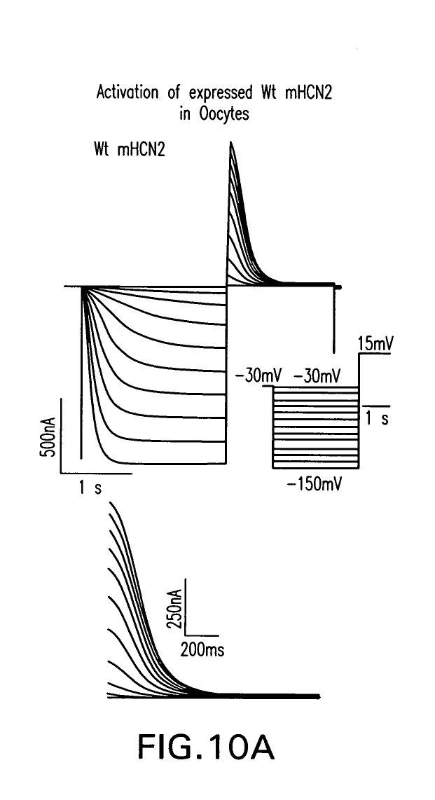

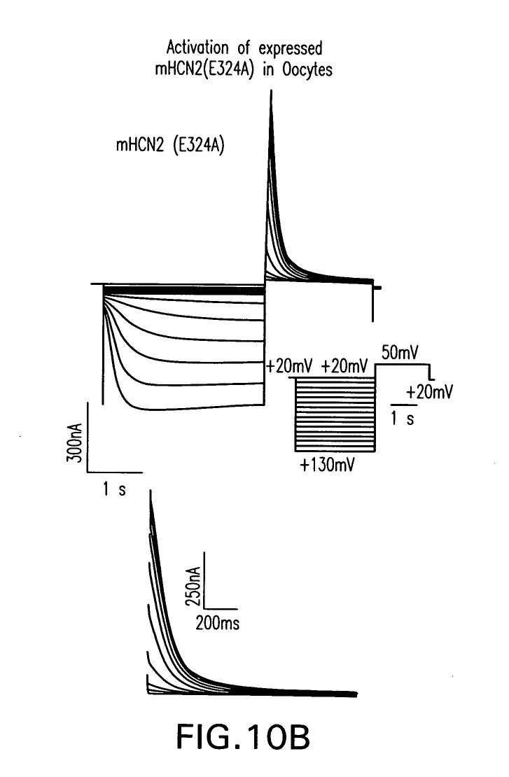

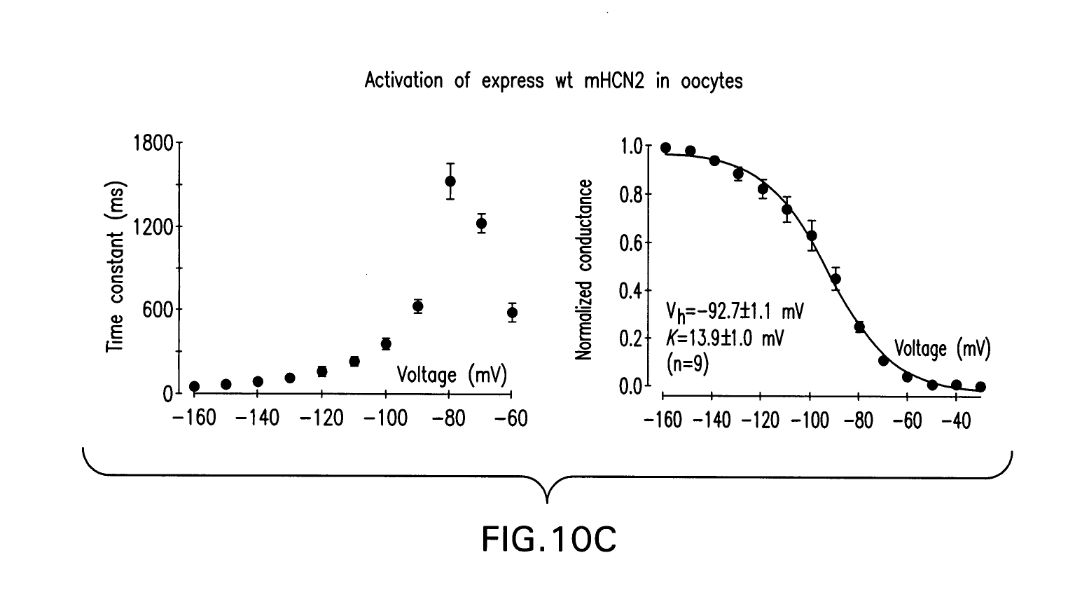

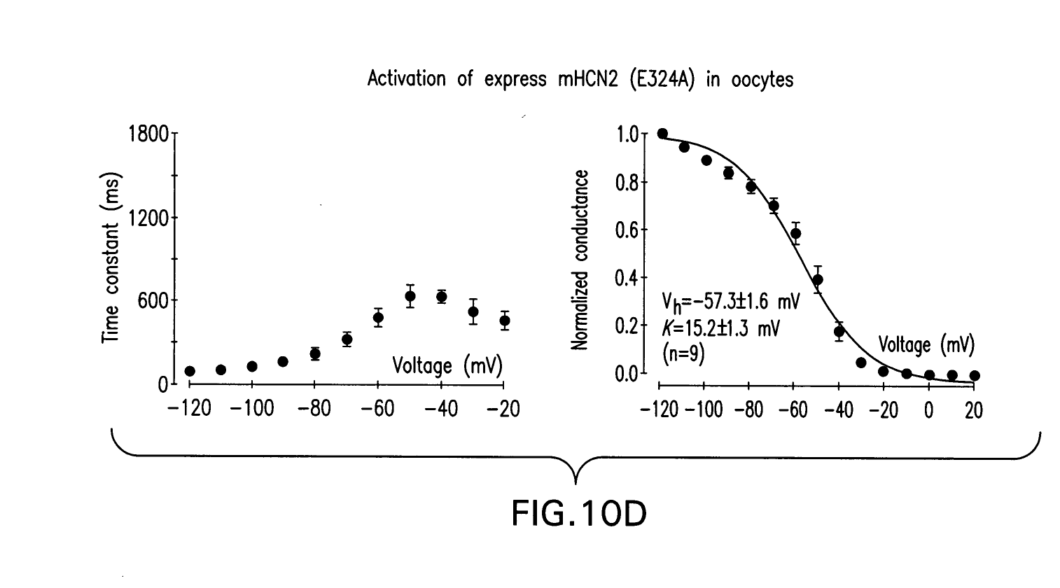

The connexins, Cx43 and Cx40, were immunolocalized, as evidenced by typical punctate staining, along regions of intimate cell-to-cell contact and within regions of the cytoplasm of the hMSCs grown in culture as monolayers (FIGS. 10A, B). Cx45 staining was also detected, but unlike that of Cx43 or Cx40, was not typical of connexin distribution in cells. Rather, it was characterized by fine granular cytoplasmic and reticular-like staining with no readily observed membrane-associated plaques (FIG. 10C). This does not exclude the possibility that Cx45 channels exist but does imply that their number relative to Cx43 and Cx40 homotypic, heterotypic and heteromeric channels is low. FIG. 10D illustrates Western blot analysis for canine ventricle myocytes and hMSCs with a Cx43 polyclonal antibody which adds further proof of Cx43 presence in hMSCs.

Gap Junctional Coupling Between hMSCs and Various Cell Lines

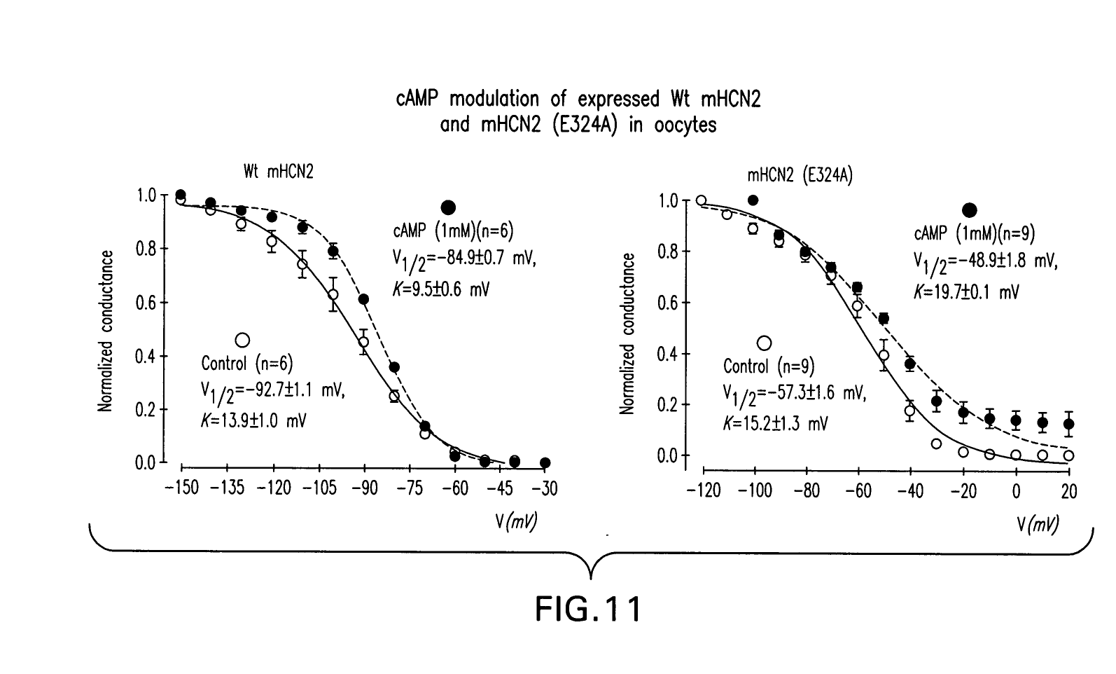

Gap junctional coupling among hMSCs is demonstrated in FIG. 11. Junctional currents recorded between hMSC pairs show quasi-symmetrical (FIG. 11A) and asymmetrical (FIG. 11B) voltage dependency arising in response to symmetrical 10-s transjunctional voltage steps (Vⱼ) of equal amplitude but opposite sign starting from ±10 mV to ±110 mV using increments of 20 mV. These behaviors are typically observed in cells which co-express Cx43 and Cx40 (Valiunas et al., 2001).

FIG. 11C summarizes the data obtained from hMSC pairs. The values of normalized instantaneous (g_{j,inst}, ∘) and steady state conductances (g_{j,ss}, •) (determined at the beginning and at the end of each Vⱼ step, respectively) were plotted versus Vⱼ. The left panel shows a quasi-symmetrical relationship from five hMSC pairs. The continuous curves represent the best fit of data to the Boltzmann equation with the following parameters: half-deactivation voltage, V_{j,0}=−70/65 mV; minimum gⱼ, g_{j,min}=0.29/0.34; maximum gⱼ, g_{j,max}=0.99/1.00; gating charge, z=2.2/2.3 for negative/positive Vⱼ, respectively. Summarized plots from six asymmetrical cases are shown in the right panel. The g_{j,ss} declined in sigmoidal fashion at negative Vⱼ and showed a reduced voltage sensitivity to positive Vⱼ. Boltzman fitting for negative Vⱼ revealed the following values: V_{j,0}=−72 mV, g_{j,min}=0.25, g_{j,max}=0.99, z=1.5.

FIGS. 11D and E illustrate typical multichannel recordings from a hMSC pair. Using 120 mM K aspartate as a pipette solution, channels were observed with unitary conductances of 28-80 pS range. Operation of channels with ˜50 pS conductance (see FIG. 11D) is consistent with previously published values (Valiunas et al., 1997; 2002) for Cx43 homotypic channels. This does not preclude the presence of other channel types, it merely suggests that Cx43 forms functional channels in hMSCs.

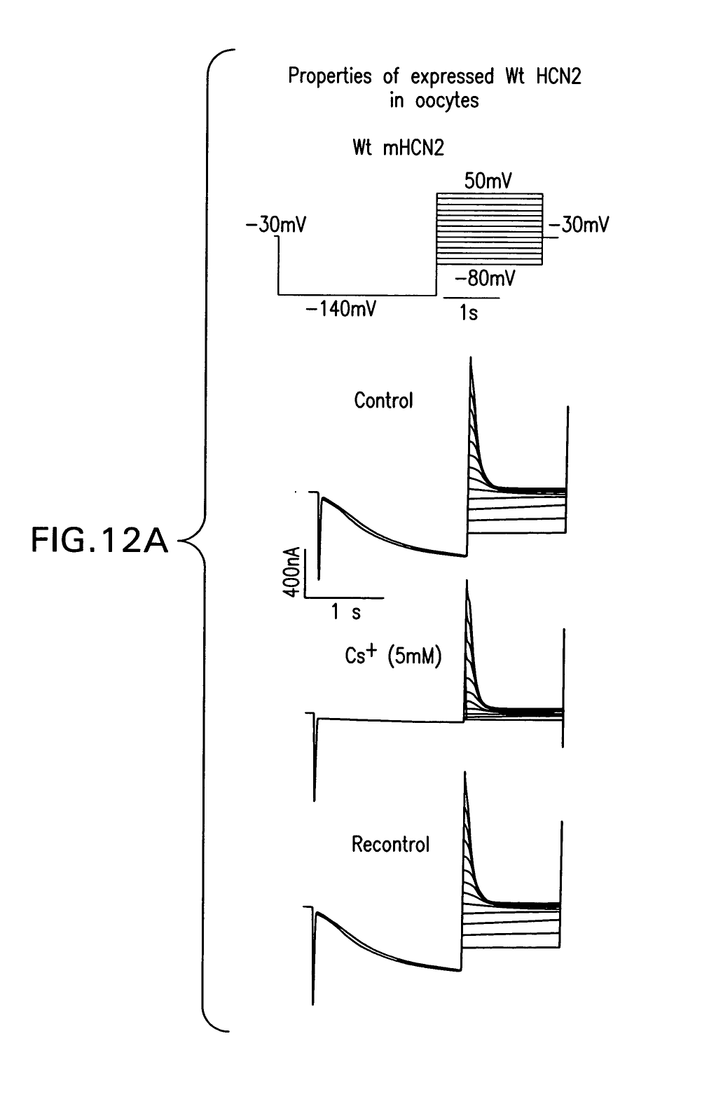

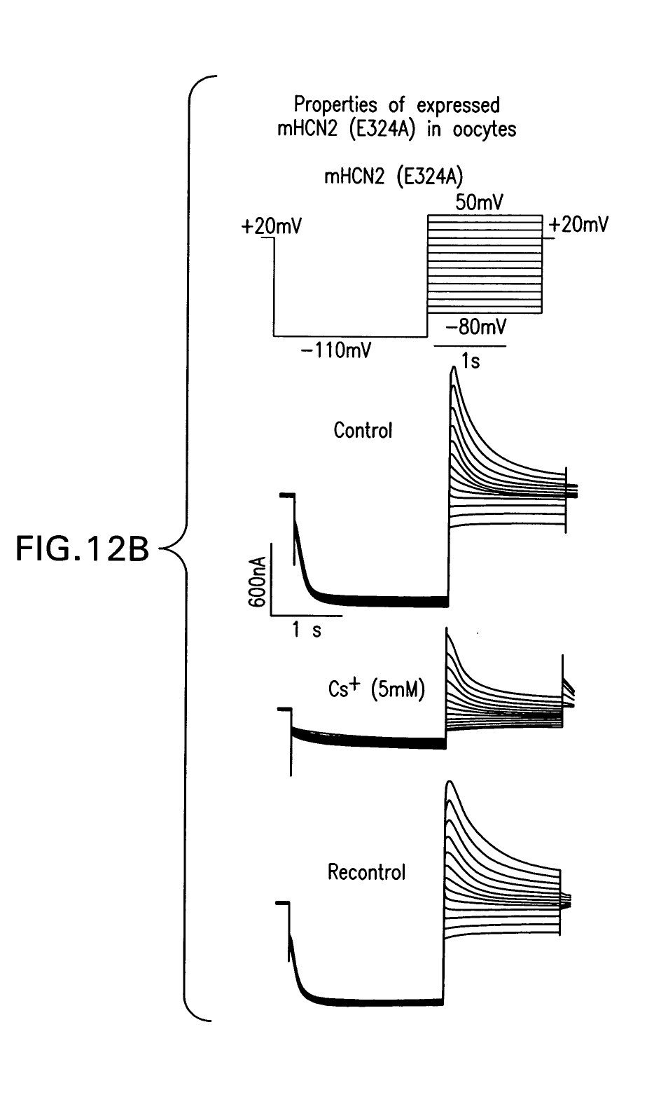

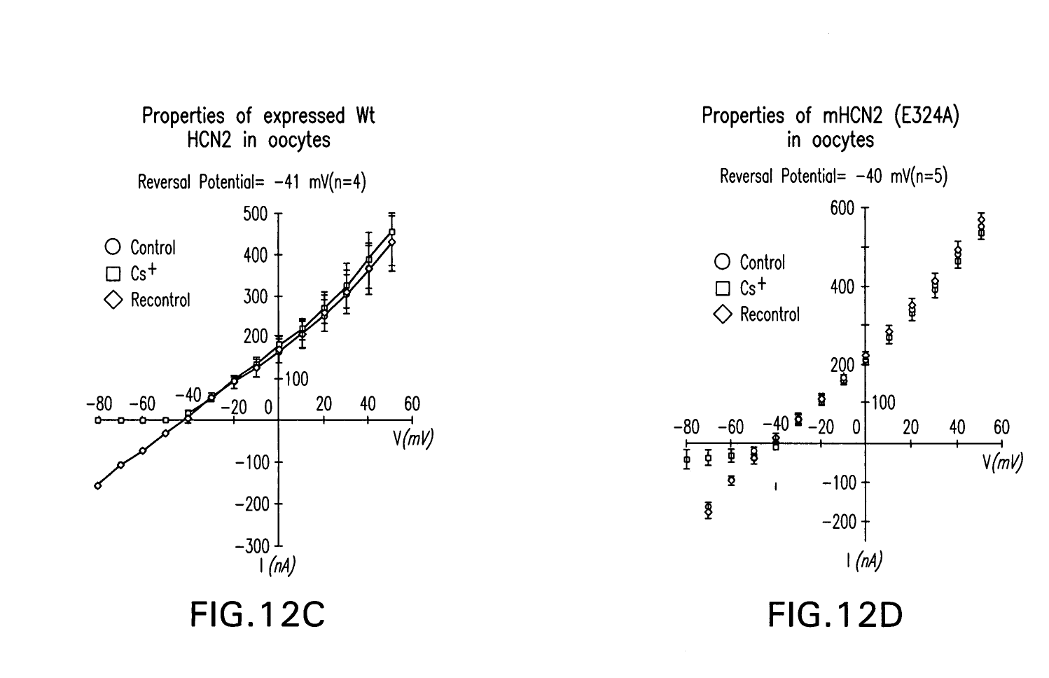

To further define the nature of the coupling, hMSCs were co-cultured with human HeLa cells stably transfected with Cx43, Cx40, and Cx45 (Elfgang et al., 1995) and it was found that hMSCs were able to couple to all these transfectants. FIG. 12A shows an example of junctional currents recorded between an hMSC and HeLaCx43 cell pairs that manifested symmetrically and asymmetrically voltage dependent currents in response to a series (from ±10 mV to ±110 mV) of symmetrical transjunctional voltage steps (Vⱼ). The quasi-symmetric record suggests that the dominant functional channel is homotypic Cx43 while the asymmetric record suggests the activity of another connexin in the hMSC (presumably Cx40 as shown by immunohistochemistry, see FIG. 10) that could be either a heterotypic or heteromeric form or both. These records are similar to those published for transfected cells: heterotypic and mixed (heteromeric) forms of Cx40 and Cx43 (Valiunas et al., 2000; 2001). Co-culture of hMSCs with HeLa cells transfected with Cx40 (FIG. 12B) also revealed symmetric and asymmetric voltage dependent junctional currents consistent with the co-expression of Cx43 and Cx40 in the hMSCs similar to the data for Cx43 HeLa-hMSC pairs. HeLa cells transfected with Cx45 coupled to hMSCs always produced asymmetric junctional currents with pronounced voltage gating when Cx45 (HeLa) side was negative (FIG. 12C). This is consistent with the dominant channel forms in the hMSC being Cx43 and Cx40 as both produce asymmetric currents when they form heterotypic channels with Cx45 (Valiunas et al., 2000; 2001). This does not exclude Cx45 as a functioning channel in hMSCs but it does indicate that Cx45 is a minor contributor to cell to cell coupling in hMSCs. The lack of visualized plaques in the immunostaining for Cx45 (FIG. 10) further supports this interpretation.

The summarized plots of g_{j,ss} versus Vⱼ from pairs between hMSC and transfected HeLa cells are shown in FIG. 12D. The left panel shows the results from hMSC-HeLaCx43 pairs. For symmetrical data (•, four preparations), Boltzmann fits (continuous lines) yielded the following parameters: V_{j,0}=−61/65 mV, g_{j,min}=0.24/0.33, g_{j,max}=0.99/0.99, z=2.4/3.8 for negative/positive Vⱼ. For asymmetrical data (∘, three preparations), the Boltzmann fit (dashed line) at negative Vⱼ values revealed the following parameter values: V_{j,0}=−70 mV, g_{j,min}=0.31, g_{j,max}=1.00, z=2.2. The middle panel shows data from hMSC-HeLaCx40 pairs including three symmetrical (•) and two asymmetrical (∘) g_{j,ss}-Vⱼ relationships. The continuous lines correspond to a Boltzmann fit to symmetrical data (V_{j,0}=−57/76 mV, g_{j,min}=0.22/0.29, g_{j,max}=1.1/1.0, z=1.4/2.3; negative/positive Vⱼ) and the dashed line is a fit to the asymmetrical data (V_{j,0}=−57/85 mV, g_{j,min}=0.22/0.65, g_{j,max}=1.1/1.0, z=1.3/2.2; negative/positive Vⱼ). The data from the six complete experiments from hMSC-HeLaCx45 cell pairs are shown on the right panel. The g_{j,ss} plot versus Vⱼ was strongly asymmetrical and the best fit of the data to the Boltzmann equation at positive Vⱼ values revealed following parameter values: V_{j,0}=31 mV, g_{j,min}=0.07, g_{j,max}=1.2, z=1.8.

FIG. 12E shows Lucifer Yellow transfer from an hMSC to an hMSC (upper panel), from a HeLaCx43 to an hMSC (middle panel), and from an hMSC to a HeLaCx43 (bottom panel). The junctional conductance of the cell pairs was simultaneously measured by methods described earlier (Valiunas et al., 2002) and revealed conductances of ˜13, ˜16 and ˜18 nS, respectively. The transfer of Lucifer Yellow was similar to that previously reported for homotypic Cx43 or co-expressed Cx43 and Cx40 in HeLa cells (Valiunas et al., 2002). Cell Tracker Green (Molecular Probes) was always used in one of the two populations of cells to allow heterologous pairs to be identified (Valiunas et al., 2000). Lucifer Yellow was always delivered to the cell containing cell tracker. The fluorescence intensity generated by the Cell Tracker Green was 10-15 times less than fluorescence intensity produced by the concentration of Lucifer Yellow delivered to the source cell.

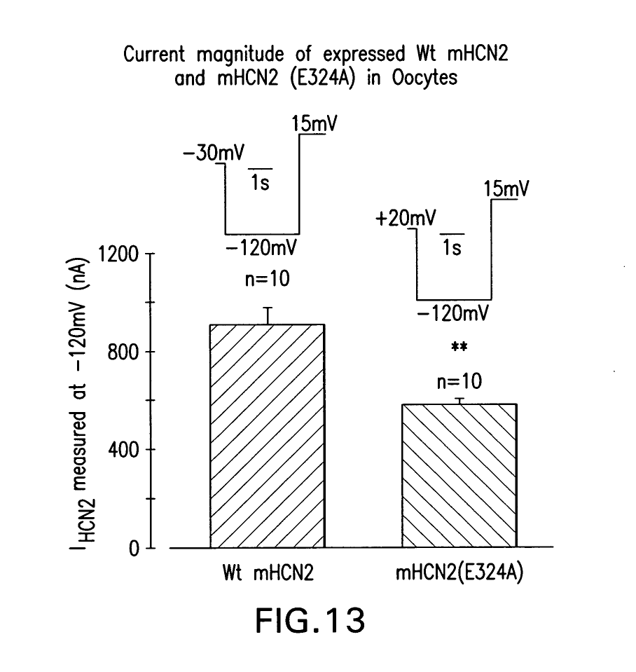

Human MSCs were also co-cultured with adult canine ventricular myocytes as shown in FIG. 13. Immunostaining for Cx43 was detected between the rod-shaped ventricular myocytes and hMSCs as shown in FIG. 13A. The hMSCs couple electrically with cardiac myocytes. Both macroscopic (FIG. 13B) and multichannel (FIG. 13C) records were obtained. Junctional currents in FIG. 13B are asymmetrical while those in FIG. 13C show unitary events of the size range typically resulting from the operation of homotypic Cx43 or heterotypic Cx43-Cx40 or homotypic Cx40 channels (Valiunas et al., 2000; 2001). Heteromeric forms are also possible whose conductances are the same or similar to homotypic or heterotypic forms.

The studies of cell pairs have demonstrated effective coupling of hMSC to other hMSC (13.8±2.4 nS, n=14), to HeLaCx43 (7.9±2.1 nS, n=7), to HeLaCx40 (4.6±2.6 nS, n=5), to HeLaCx45 (11±2.6 nS, n=5.), and to ventricular myocytes (1.5±1.3 nS, n=4).

Use of hMSCs as a Delivery Platform for Biological Pacemaking

Human MSCs are viewed as a favorable platform candidate for delivering biological pacemakers into the heart partly on the basis, suggested by Liechty et al. (2000), that they might be immunoprivileged and as such would hopefully not give rise to a rejection response. This is important because in the tradeoff between biological and electronic pacemakers, any need for immunosuppression using the former approach would be a detriment to cell therapy approaches and clinically undesirable.

Human MSCs are obtained readily commercially or from the bone marrow, and are identified by the presence of CD44 and CD29 surface markers, as well as by the absence of other markers that are specific for hematopoietic progenitor cells. Using a gene chip analysis, it was determined that the hMSCs do not carry message for HCN isoforms. Importantly, they also do have a significant message level for the gap junctional protein, connexin 43. The latter observation is critical because the theory behind platform therapy is that the hMSC would be loaded with the gene of interest, e.g., HCN2, and implanted into myocardium (Rosen et al., 2004). However, having a cell loaded with a signal would not work unless the cell formed functional connections with its neighbors. The philosophy underlying the use of hMSCs as a delivery platform is summarized in FIG. 1. In brief, in the normal sinus node, hyperpolarization of the membrane initiates inward (I_{f}) current which generates phase 4 depolarization and an automatic rhythm. The changes in membrane potential result in current flow via the low resistance gap junctions such that the action potential propagates from one cell to the next. Use of the hMSC as a platform involves loading it with the gene of interest, e.g., HCN2, preferably via electroporation, thereby avoiding any viral component of the process (Rosen et al., 2004; Rosen, 2005; Cohen et al., 2005; Potapova et al., 2004). The hMSC would have to be coupled effectively to the adjacent myocyte. If this occurred, then the high negative membrane potential of coupled myocytes would hyperpolarize the hMSC, opening the HCN channel and permitting inward current to flow. This current, in turn, would propagate though the low resistance gap junctions, depolarize a coupled myocyte and bring it to threshold potential, resulting in an action potential that would then propagate further in the conducting system. In other words, the hMSC and the myocyte each would have to carry an essential piece of machinery: the myocyte would bring the ionic components that generate an action potential, the hMSC would carry the pacemaker current, and—if gap junctions were present—the two separate structural entities would function as a single, seamless physiologic unit.

The key question then is whether gap junctions are formed between hMSCs and myocytes. The answer is affirmative, as the experimental data disclosed above show. FIG. 10 shows that connexins 43 and 40 are clearly demonstrable in hMSCs. In addition, hMSCs form functional gap junction channels with cell lines expressing Cx43, Cx40 or Cx45 as well as with canine ventricular cardiomyocytes (see also Valiunas et al., 2004, the entire contents of which are hereby incorporated by reference). Lucifer Yellow passage between an hMSC and another hMSC or a HeLaCx43 cell (see FIG. 12E) is yet another indicator of robust gap junction-mediated coupling. The transfer of Lucifer Yellow between hMSCs and HeLa cells transfected with Cx43 is similar to that of homotypic Cx43 or coexpressed Cx43 and Cx40. It excludes homotypic Cx40 as a dominating channel type as Cx40 is some 5 times less permeable to Lucifer Yellow than Cx43 (Valiunas et al., 2002). Moreover, injection of current into an hMSC in close proximity to a myocyte results in current flow to the myocyte (FIG. 13), further indicative of the establishment of functional gap junctions.

These data suggest that MSCs should readily integrate into electrical syncytia of many tissues, promoting repair or serving as the substrate for a therapeutic delivery system. In particular, the data support the possibility of using hMSCs as a therapeutic substrate for repair of cardiac tissue. Other syncytia such as vascular smooth muscle or endothelial cells should also be able to couple to the hMSCs because of the ubiquity of Cx43 and Cx40 (Wang et al., 2001a). Thus, they may also be amenable to hMSCs-based therapeutics. For example, hMSCs can be transfected to express ion channels which then can influence the surrounding synctial tissue. Alternatively, the hMSCs can be transfected to express genes that produce small therapeutic molecules capable of permeating gap junctions and influencing recipient cells. Further, for short term therapy, small molecules can be directly loaded into hMSCs for delivery to recipient cells. The success of such approaches is dependent on gap junction channels as the final conduit for delivery of the therapeutic agent to the recipient cells. The feasibility of the first approach has been demonstrated herein by delivering HCN2-transfected hMSCs to the canine heart where they generate a spontaneous rhythm.

Another question concerned the autonomic responsiveness of the hMSCs. As shown by Potapova et al. (2004), the addition of isoproterenol to hMSCs loaded with HCN2 resulted in a shift in activation such that increased current flowed at more positive potentials. The result, as would be expected for native HCN2, should be an increased pacemaker rate. Potapova et al. (2004) also investigated the response of I_{f} expressed by hMSCs to acetylcholine. Acetylcholine alone had no effect on current, but in the presence of isoproterenol antagonized the beta-adrenergic effect of the latter. This is entirely consistent with the physiologic phenomenon of accentuated antagonism.

Human MSCs loaded with HCN2 were also injected into the hearts of dogs in which vagal stimulation was used to terminate sinoatrial pacemaker function and/or atrioventricular conduction (Potapova et al., 2004). This resulted in spontaneous pacemaker function that was pace-mapped to the site of injection. Moreover, tissues removed from the site showed gap junctional formation between myocyte and hMSC elements. Finally, the stem cells stained positively for vimentin, indicating that they were mesenchymal, and positively for human CD44 antigen, indicating that they were hMSCs of human origin (Potapova et al. 2004).

In a preliminary study, Plotnikov et al. (2005b) followed the function of hMSC-based biological pacemaking through six weeks post-implantation and found that the rate generated is stable. Equally importantly, staining for immune globulin and for canine lymphocytes was used to determine if rejection of the hMSCs was occurring. Using 2-week and 6-week time points, there was no evidence for humoral or cellular rejection. This is consistent with the earlier work of Liechty et al (2000) suggesting that hMSCs may be immunoprivileged. If more detailed investigation demonstrates this to be the case, then it would abrogate any need for immunosuppression.

Overall, therefore, hMSCs appear to provide a very attractive platform for delivering pacemaker ion channels to the heart for several reasons: they can be obtained in relatively large numbers through standard clinical interventions; they are easily expanded in culture; preliminary evidence suggests they are capable of long-term transgene expression; and their administration can be autologous or via banked stores (as they are immunoprivileged). Whereas hMSCs might in theory be differentiated in vitro into cardiac-like cells capable of spontaneous activity, the genetic engineering approach described herein does not depend on differentiation along a specific lineage. Moreover, this ex vivo transfection method allows evaluation of DNA integration and engineering of the cell carriers with fail-safe death mechanisms. Accordingly, adult hMSCs are a preferred ion channel delivery platform to be employed in methods for treating subjects afflicted with cardiac rhythm disorders comprising the induction of biological pacemaker activity in the subject's heart, and in making kits for use in such methods.

It is important to emphasize the conceptual and practical differences between the design of (1) gene therapy, and (2) stem cell therapy as described herein. Whereas both have one endpoint in common—the delivery of a biological pacemaker—gene therapy uses specific HCN isoforms to engineer a cardiac myocyte into a pacemaker cell, whereas hMSC therapy uses stem cells as a platform to carry specific HCN and/or MiRP1 isoforms to a heart whose myocytes retain their original function. Gene therapy makes use of preexisting homeotypic cell-cell coupling among myocytes to facilitate propagation of the pacemaker impulses from those myocytes in which pacemaker current is overexpressed to those that retain their original function. In contrast, stem cells depend on heterotypic coupling of cells with somewhat dissimilar populations of connexins to deliver pacemaker current alone from a stem cell to a myocyte whose function is left unchanged. Importantly, and unlike sinus node cells, HCN2-transfected hMSCs are not excitable, because they lack the other currents necessary to generate an action potential. However, when transfected, these cells generate a depolarizing current, which spreads to coupled myocytes, driving myocytes to threshold. In effect, the myocyte acts like a trip wire whose hyperpolarization turns on pacemaker current in the stem cell and whose depolarization turns off the current. The data presented herein suggest that as long as the hMSCs contain the pacemaker gene and couple to cardiac myocytes via gap junctions, they will function as a cardiac pacemaker in an analogous manner to the normal primary pacemaker the sinoatrial node.

Mass of Biological Pacemaker Required for Normal Pacemaker Function

A biological pacemaker needs an optimal size (in terms of cell mass) and an optimal cell-to-cell coupling for long-term normal function. It was fortuitous in the early studies that the HCN constructs used, and the number of transfected hMSCs administered to the canine heart in situ, coupled to surrounding myocytes and functioned as well as they did to generate significant, easily measurable pacemaker activity. A mathematical model has subsequently been used to identify the appropriate hMSC numbers and coupling ratios needed to optimize function.

The mathematical model was used to reconstruct an in vivo stem cell injection using quantum dot nanoparticles (QD). Approximately 120,000 QD-containing hMSCs were injected into rat LV free wall (at z=4.9 mm), and the animal was terminated 1 h after injection. Transverse 10-μm sections were cut and visualized for QD fluorescence at 655 nm with phase contrast overlay to show tissue borders. QD were found within the delivered hMSCs and single QD⁺-cells were visualized in the myocardium at higher resolutions. QD⁺-regions from 230 serial 10-μm transverse sections were identified and used to reconstruct the 3D distribution of QD clusters in the heart. A biological pacemaker was then mathematically modeled taking into account the properties of I_{f} in a stem cell, the effects of cell geometry on the propagation of an action potential, the number of stem cells, the resting-voltage-induced reductions of I_{f}, and the requirements for propagation of an action potential. The radius of a hMSC was assumed to be 7 μm, which meant that the radius of a cluster of 10⁵ stem cells is 0.03 cm, and 0.07 cm for 10⁶ stem cells.

The model indicated that: 10⁵ or more stem cells would generate a muscle action potential; the characteristic input resistance of muscle saturates at about 0.03 cm; because of voltage-dependent reductions in I_{f}, current leaving the stem cell cluster saturates at about 0.03 cm and thus the pacemaker potential in muscle saturates at about 0.03 cm. It was concluded that self sustaining propagation of an action potential in muscle is essentially guaranteed if a shell of cells of radius of about 0.03 cm or larger reaches threshold. This implies that if 1,000,000 stem cells are injected, only 10% need to survive to create a biological pacemaker. These conclusions are consistent with the experimental results on the induction of pacemaker activity in heart tissue in situ disclosed herein.

### EXAMPLE 4

### Use of Chimeric HCN Channels for Biological Pacemaking

Chimeric HCN Channel Constructs

For constructing HCN chimeras, the HCN genes are first subcloned into expression vectors. For example, mammalian genes encoding HCN1-4 (Santoro et al., 1998; Ludwig et al., 1998; 1999; Ishii et al., 1999) are subcloned into vectors such as pGH19 (Santoro et al., 2000) and pGHE (Chen et al., 2001b). Deletion and chimeric mutants are then made by a PCR/subcloning strategy, and the sequences of the resulting mutant HCN constructs are verified by DNA sequencing.

HCN channels can be characterized as having three main portions, a hydrophilic, cytoplasmic N-terminal portion (region 1), a six-membered, S1-S6 core membrane-spanning (intramembrane) portion (region 2) comprising mainly hydrophobic amino acids, and a hydrophilic, cytoplasmic C-terminal portion (region 3). The boundaries of these portions can readily be determined by one of ordinary skill in the art based on the primary structure of the protein and the known hydrophilicity or hydrophobicity of the constituent amino acids. For example, in mHCN1, the C-terminal portion is D390-L910. The C-terminal portion of mHCN2 is D443-L863. Polynucleotide sequences encoding the entire N-terminal domain, the core transmembrane domain, or the C-terminal domain from any of HCN1, HCN2, HCN3 and HCN4, can be interchanged. The different chimeras so constructed are identified using the nomenclature HCNXYZ, where X, Y, or Z is a number (either 1, 2, 3 or 4) that refers to the identity of the N-terminal domain, core transmembrane domains, or C-terminal domain, respectively.

Thus, for example, in the mHCN112 chimera (see FIG. 3), the N-terminal and the intramembrane portions are from mHCN1 whereas the C-terminal amino acids D390-L910 of mHCN1 are substituted by the carboxy-terminal amino acids D443-L863 of mHCN2. Conversely, in mHCN221, the carboxy-terminal amino acids D443-L863 of mHCN2 are substituted by the carboxy-terminal amino acids D390-L910 of mHCN1. In mHCN211, the amino terminal amino acids M1-S128 of mHCN1 are substituted the amino terminal amino acids M1-S181 of mHCN2. Conversely, in mHCN122, amino acids M1-S181 of mHCN2 are substituted by M1-S128 of mHCN1. In mHCN121, the S1-S6 transmembrane domain amino acids D129-L389 of mHCN1 are substituted by the transmembrane domain amino acids D182-L442 of mHCN2. Conversely, in mHCN212 (FIG. 3), amino acids D182-L442 of HCN2 (i.e., the intramembrane portion) are substituted by D129-L389 of mHCN1 (see Wang et al., 2001b). For preparing human chimeric HCN channels, the same principles are applied mutatis mutandis, employing domains from human HCN channels. For example, hHCN112 has an amino terminal domain and an intramembrane domain from hHCN1, and a carboxy terminal domain from hHCN2.

Expression of these HCN chimeras is readily observable in Xenopus oocytes. For example, cRNA can be transcribed from NheI-linearized DNA (for HCN1 and mutants based on the HCN1 background) or SphI-linearized DNA (for HCN2 and mutants based on the HCN2 background) using a T7 RNA polymerase (Message Machine; Ambion, Austin, Tex.). 50 ng of cRNA is injected into Xenopus oocytes as described previously (Goulding et al., 1992).

Chimeric HCN Channels Enhances Biological Pacemaking

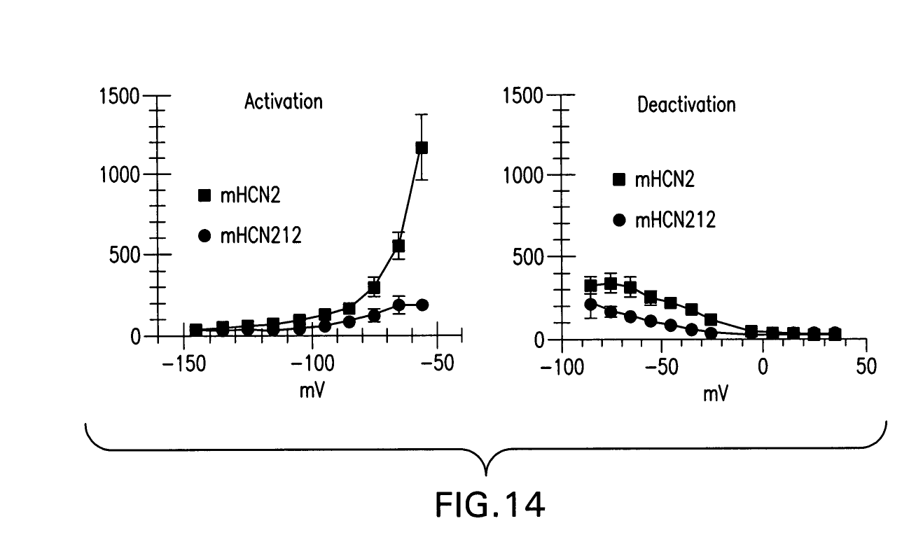

Experiments were performed to compare the gating kinetics of HCN2 and chimeric HCN212 channels when expressed in neonatal rat ventricular myocytes. FIG. 14 shows the results obtained using mHCN2 and a chimeric channel (mHCN212) created by substituting D182-L442 of murine HCN2 with D 129-L389 of murine HCN1. Analysis of the activation and deactivation kinetics reveals that mHCN212 exhibits faster kinetics at all voltages compared to mHCN2.

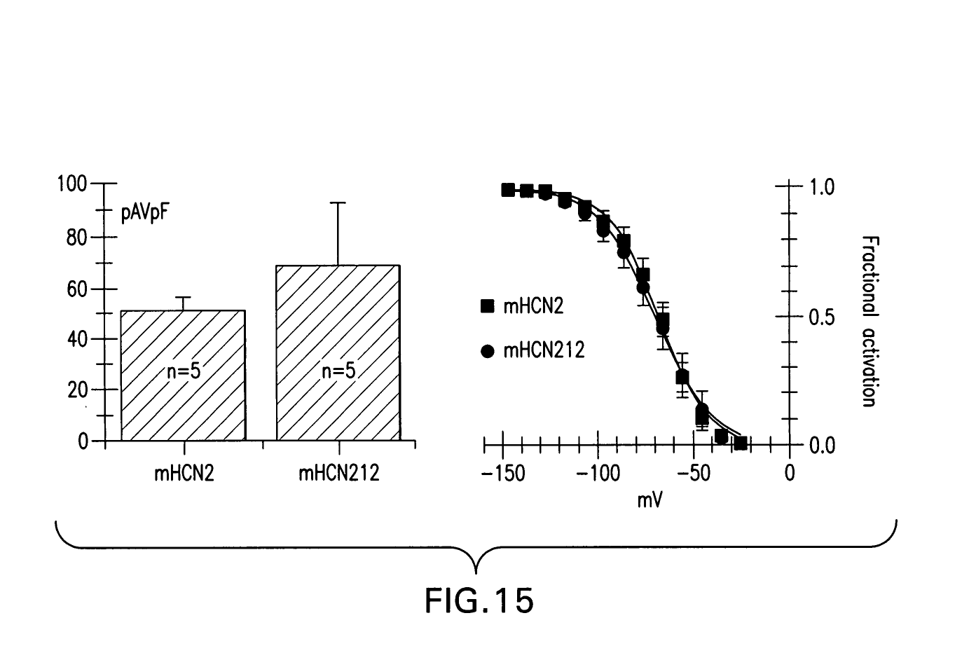

A comparison of expression efficiency of HCN2 and chimeric HCN212 channels in neonatal rat ventricular myocytes is shown in FIG. 15. The results indicate that the expression of the chimeric channel is at least as good as that of the wild-type channel. Moreover, analysis of the voltage dependence of activation indicates no difference in voltage dependence of HCN2 and HCN212 channels when expressed in myocytes.

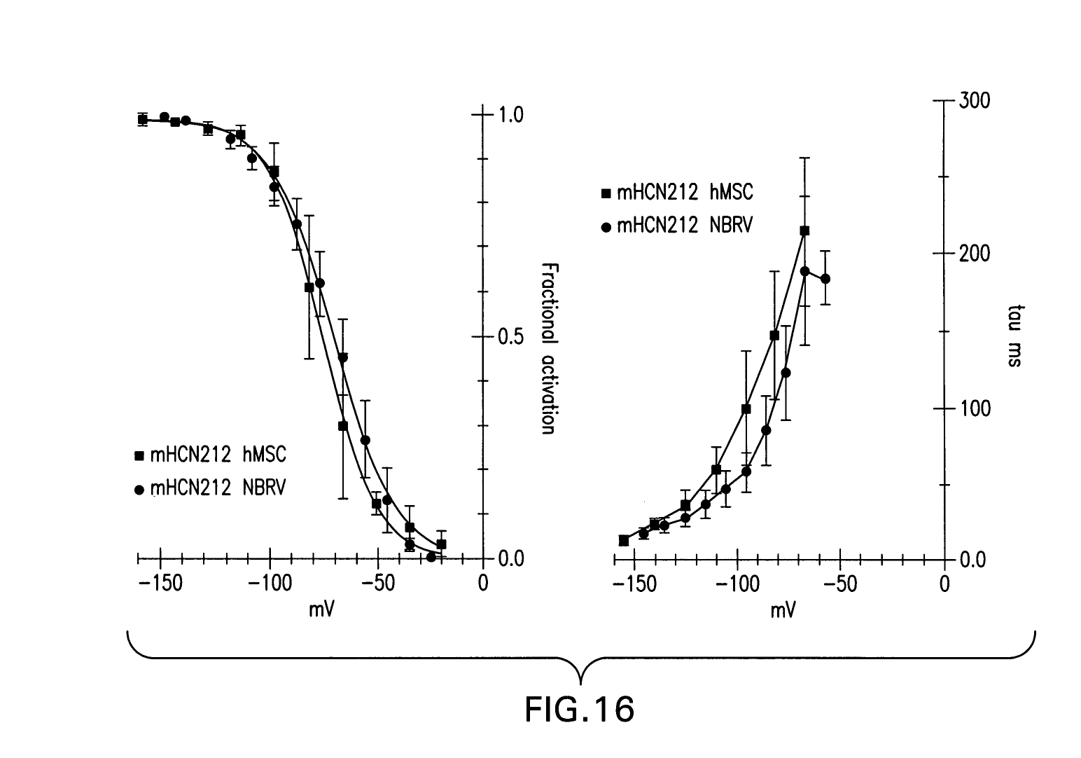

Murine HCN212 was expressed in neonatal rat ventricular myocytes and human adult mesenchymal stem cells and the expressed current subsequently studied in culture. There is no significant difference in the voltage dependence of activation or the kinetics of activation when the chimeric mHCN212 channel is expressed in the two different cell types (see FIG. 16).

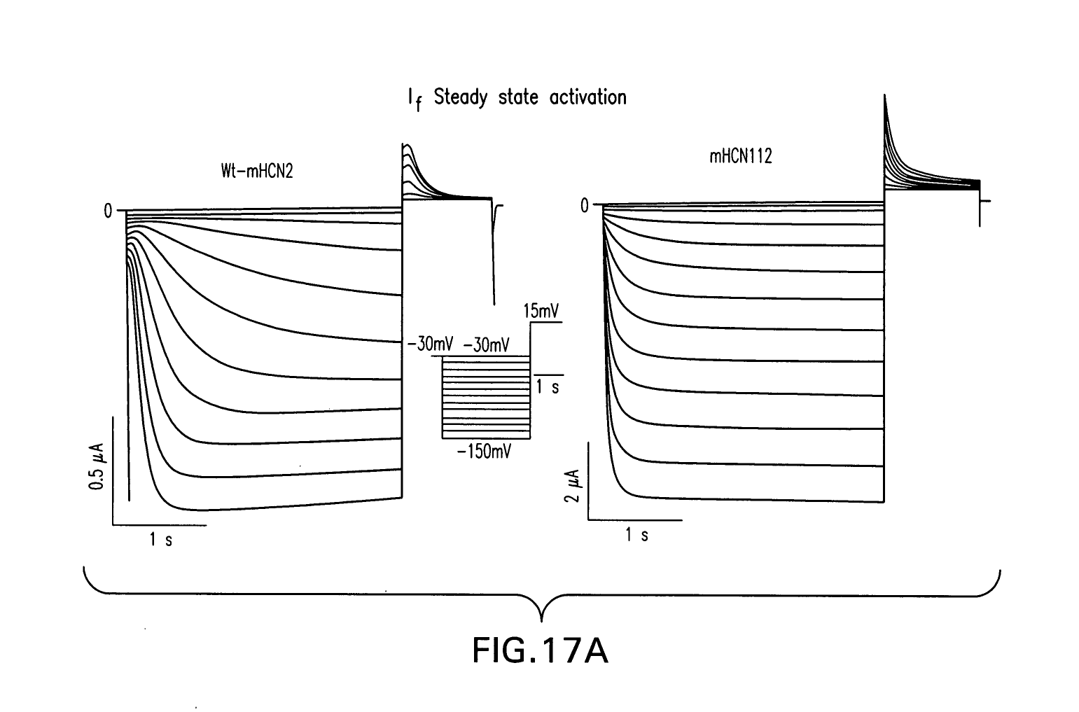

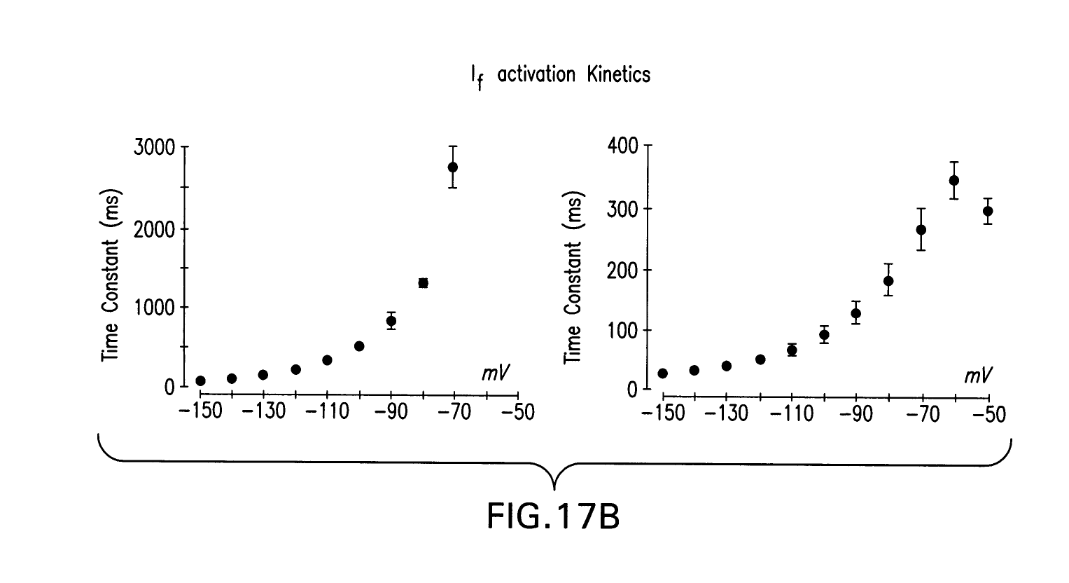

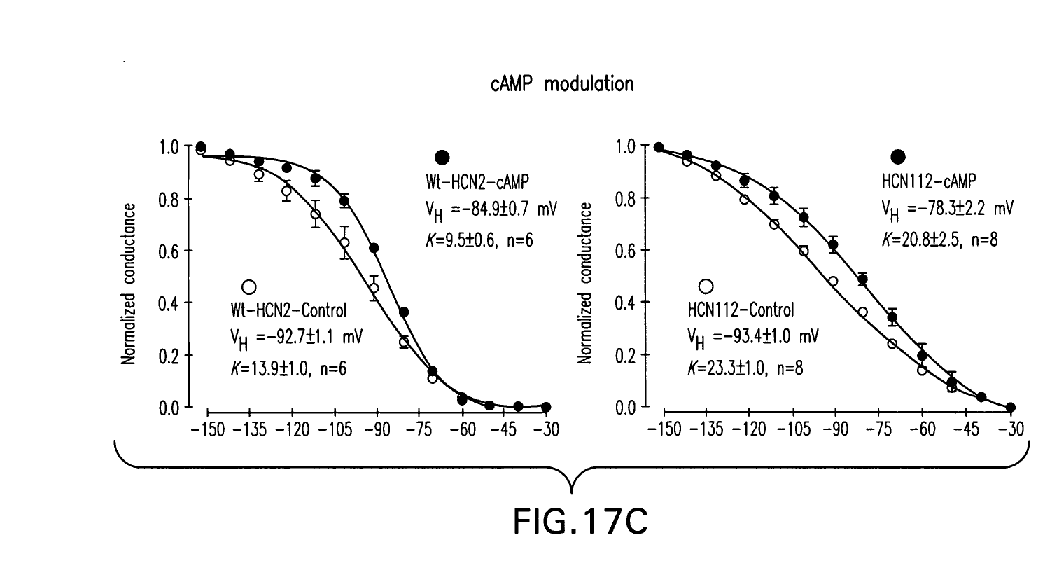

FIG. 17 shows the steady state activation curve, activation kinetics and cAMP modulation of wildtype mHCN2 and mHCN112 in oocytes. The data illustrate that the chimeric HCN112 channel achieves significantly faster kinetics than HCN2 while preserving a strong cAMP response.

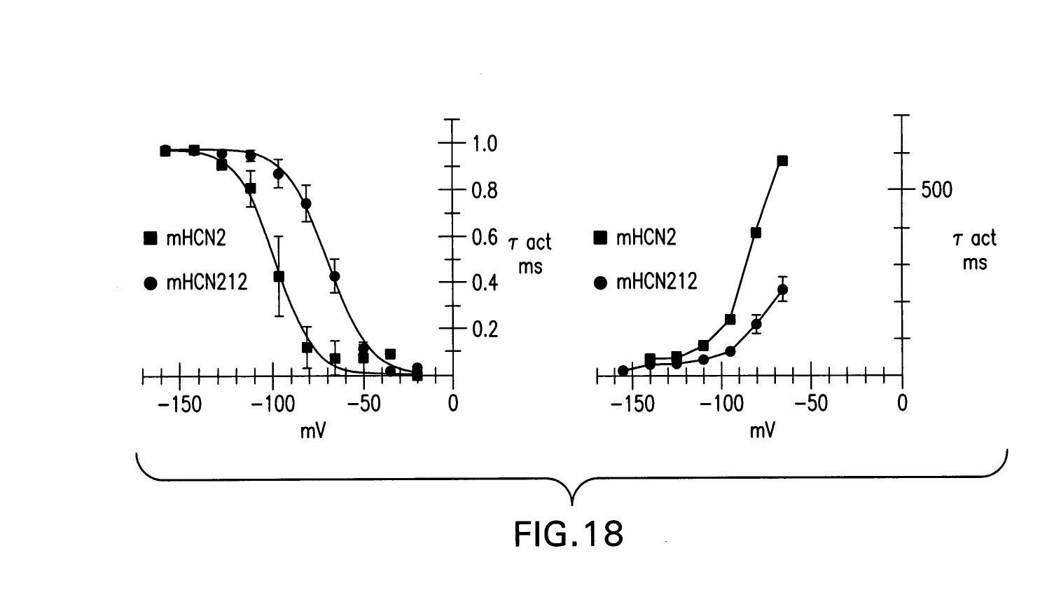

A comparison of the gating characteristics of mHCN2 and chimeric mHCN212 channels expressed in adult hMSCs (FIG. 18) shows that the voltage dependence of activation is shifted significantly positive, and the kinetics of activation at any measured voltage are significantly faster, for mHCN212 compared to HCN2.

These data suggest that the HCN212 chimera has significant advantages over the wild-type HCN2 channel in inducing pacemaker activity for therapeutic applications. Importantly, the positive shift and faster kinetics would be expected to result in more current at shorter times for any specific voltage, and in particular, for voltages in the diastolic potential range of cardiac cells (−50 to −90 mV).

Thus, manipulations can be employed to create chimeric HCN channels that have suppressed or enhanced activities compared to the native HCN channels from which they were derived, which allows selection of channels with different characteristics optimized for treating cardiac conditions. For example, the activation curves of the HCN channel current may be shifted to more positive or more negative potentials; the hyperpolarization gating may be enhanced or suppressed; the sensitivity of the channel to cyclic nucleotides may be increased or decreased; and differences in basal gating may be introduced. More particularly, the data provide evidence that a pacemaker channel with fast kinetics and good responsiveness to cAMP (and hence altered responsiveness to autonomic stimulation) can be obtained by, for example, selection of HCN1 components. Slower kinetics may also be obtained by, for example, selection of HCN4 components in the chimera. The creation of HCN chimeras exhibiting characteristics that are beneficial for treating heart disorders has not previously been reported.

### EXAMPLE 5

### Pacemaking by Tandem Biological and Electronic Pacemakers In Situ

Implantation of Tandem Biological and Electronic Pacemakers in Dogs

Experiments involving animals were performed using protocols approved by the Columbia University Institutional Animal Care and Use Committee and conform to the Guide for the Care and Use of Laboratory Animals (NIH Publication No. 85-23, revised 1996).

Adult mongrel dogs weighing 22-25 kg were anesthetized with propofol 6 mg/kg IV and inhalational isoflurane (1.5%-2.5%). Using a steerable catheter, saline (n=5), AdmHCN2 (n=6) or AdmE324A (n=4) were injected into the left bundle branch (LBB) as described previously (Plotnikov et al., 2004). In 2 additional dogs AdmE324A was injected into the LV septal myocardium as an internal control. Complete AV block was induced via radiofrequency ablation and each site of injection was paced via catheter electrode to distinguish electrocardiographically the origin of the idioventricular rhythm during the follow up period.

An electronic pacemaker (Discovery II, Flextend lead; Guidant, Indianapolis, Ind.) was implanted and set at VVI 45 bpm. ECG, 24 hour Holter monitoring, pacemaker log record check, and overdrive pacing at 80 bpm were performed daily for 14 days. To evaluate beta-adrenergic responsiveness, on day 14, epinephrine (1.0, 1.5 and 2.0 μg/kg/min for up to 10 min each) was infused to an endpoint of a 50% increase in idioventricular rate or ventricular arrhythmia (single ventricular premature beats having a morphology other than that of the dominant idioventricular rhythm or ventricular tachycardia), whichever occurred first. If none of the above responses was observed within 10 min after onset of the maximal dose of 2 μg/kg/min, the infusion was terminated.

Data are presented as means±SEM. In the in situ experiments, the 5 saline-injected dogs and the 2 injected into the myocardium (rather than the LBB) with AdmE324A showed no electrophysiologic differences and were combined into one control group for subsequent analysis. One-way ANOVA was used to evaluate the effect of an implanted construct on electrophysiological parameters. Subsequent analysis was performed using Bonferroni's test where equal variances were assumed and the Games-Howell test where variances were unequal. A two-way contingency table analysis was conducted to evaluate whether epinephrine had different effects across three groups. Data were analyzed using SPSS for Windows software (SPSS, Inc.). P<0.05 was considered to be significant.

Operation of Tandem Biological and Electronic Pacemakers In Situ

In a preliminary experiment, the possibility that injecting an adenovirus carrying the E324A mutant might provide an effective alternative to HCN2 was tested in vivo. It was found that E324A-infected dogs manifested basal rates that did not differ significantly from those of HCN2-infected animals, while their catecholamine-responsiveness was greater (Plotnikov et al., 2005a).

In the present experiments, adenoviral vectors carrying the HCN2 and E234A-HCN2 genes, respectively, were then used to generate pacemaking activity in vivo in tandem with implanted electronic pacemakers, and the performance of the tandem pacemakers was compared with that of an electronic pacemaker used alone. Six dogs received injections of an adenoviral vector incorporating the HCN2 gene in 0.6 ml of saline into the left bundle branch (LBB) via a steerable catheter. The HCN2 virus had been characterized in neonatal rat myocytes as follows: midpoint of activation=−69.3 mV (n=5); at −65 mV activation τ=639±72 ms (n=5); expressed current at −135 mV=53.5±8.3 pA/pF (n=10). Four dogs were injected with an adenoviral vector incorporating the mutant E324A gene in the LBB, and two additional dogs were injected into the LV septal myocardium as an internal control. As another control, five dogs received 0.6 ml of saline injected into the LBB.

Complete AV block was induced via radiofrequency ablation, and electronic pacemakers were implanted into the right ventricular apical endocardium and set a VV1 45 bpm. ECG and 24-h monitoring were performed daily for 14 days. Beta-adrenergic responsiveness was also evaluated as described above.

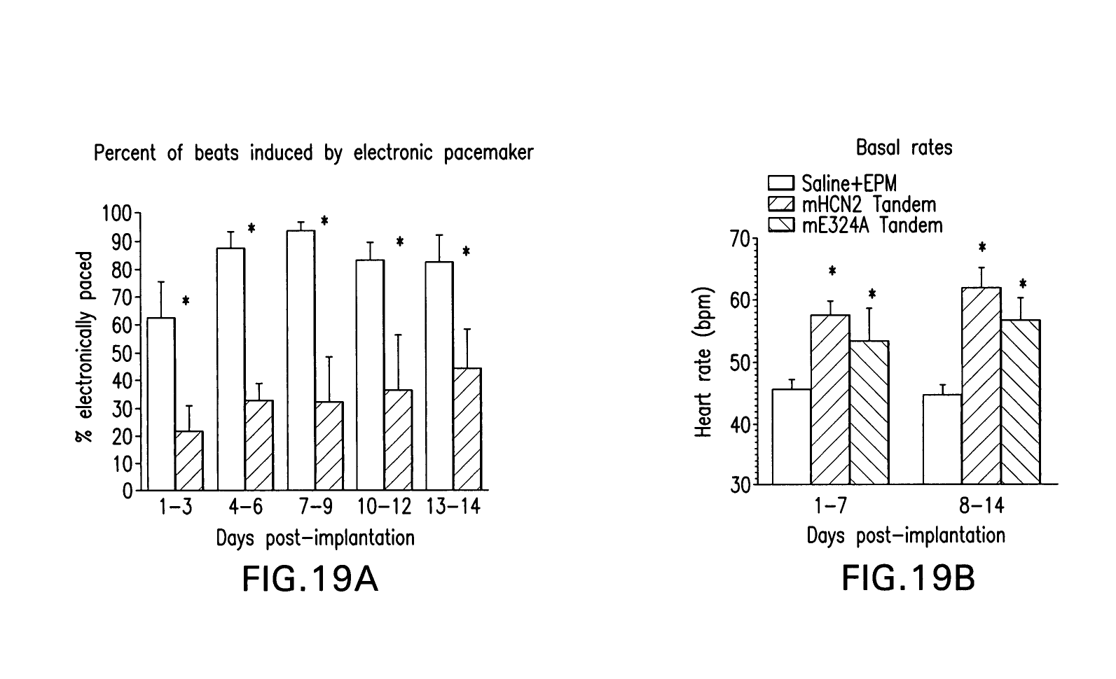

The electronic pacemaker triggered 83±5% of all beats in controls, contrasting (P<0.05) with 26±6% in the mHCN2 and 36±7% in the mE324A groups (for the latter two, P>0.05). A temporal analysis of the electronically paced beats for the tandem HCN2-electronic versus the electronic-only pacemaker is shown in FIG. 19A. It is noteworthy that a significantly lower number of beats was initiated electronically in the HCN2 group throughout the study period. Results for E324A (not shown) did not differ significantly from HCN2.

Escape time was evaluated daily by performing three 30-s periods of ventricular overdrive pacing at 80 bpm followed by an abrupt cessation of pacing. The average time between the final electronically paced beat and the first intrinsic beat was then determined. Escape times ranged from 1-5 s across all three groups and incorporated a wide variability, such that no significant differences were seen. Hence no advantage accrued to any group with regard to escape intervals. There was a different result with regard to basal heart rates throughout the 14-day period, however. As shown in FIG. 19B, average heart rate in saline controls was that determined by the rate of the electronic pacemaker (45 bpm). This was significantly slower throughout the study than that of mHCN2 or mE324A-injected dogs, which groups did not differ from one another.

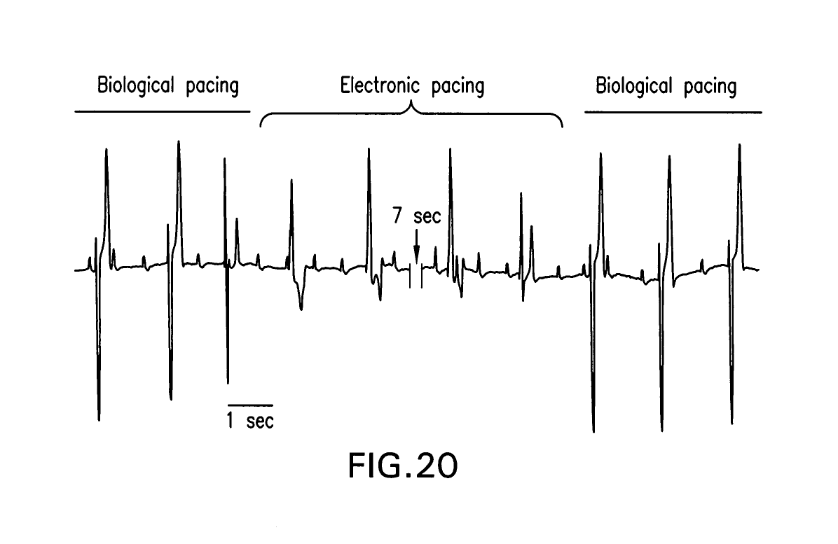

An example of the interrelationship between the biological and the electronic components of the tandem pacemaker is shown in FIG. 20. It is evident that as the biological component slows, the electronic takes over, and that as the biological component speeds in rate, the electronic ceases to fire.

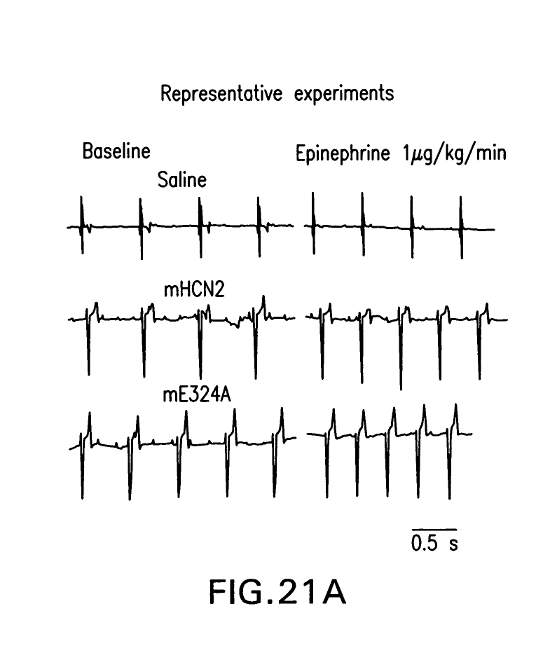

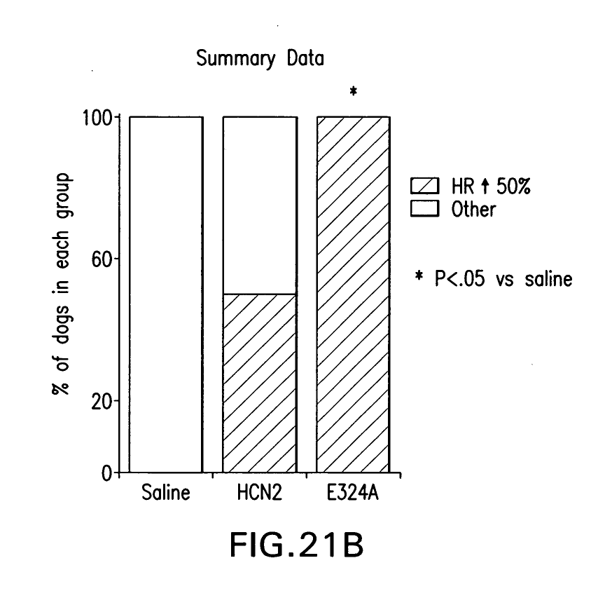

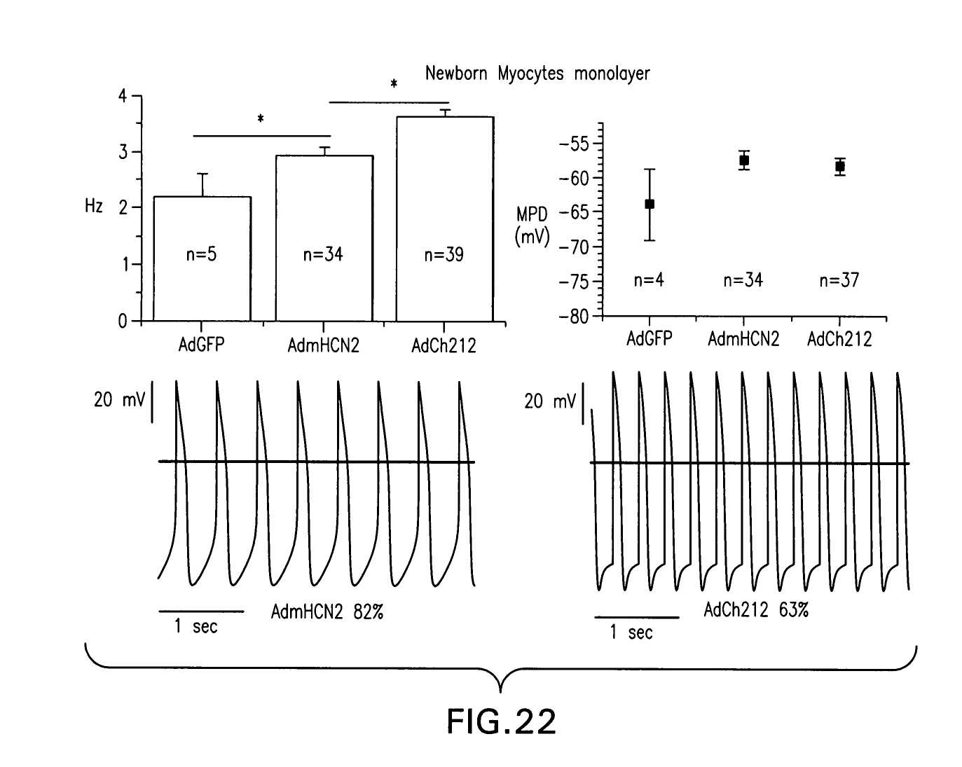

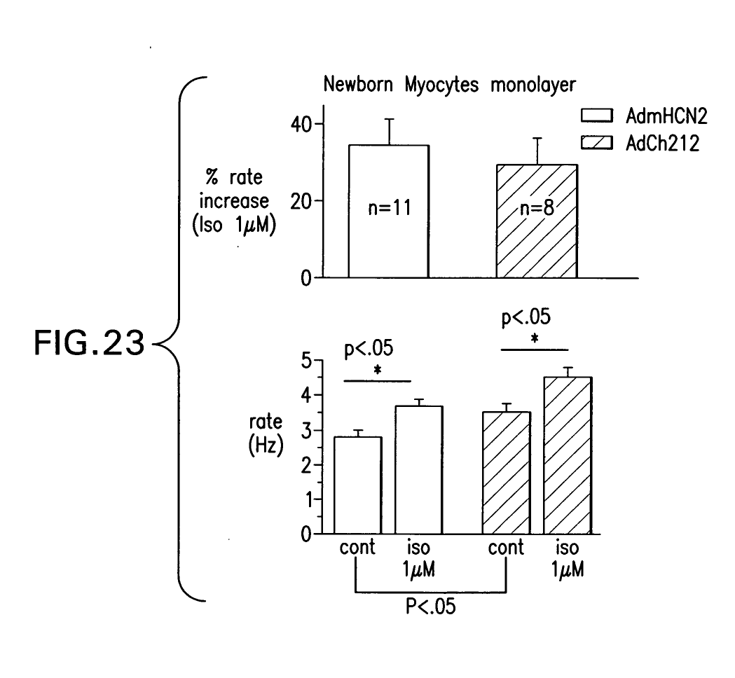

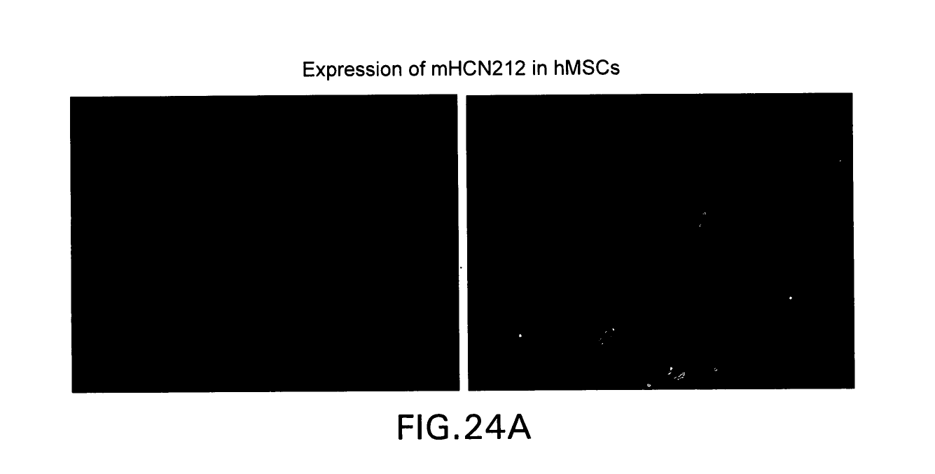

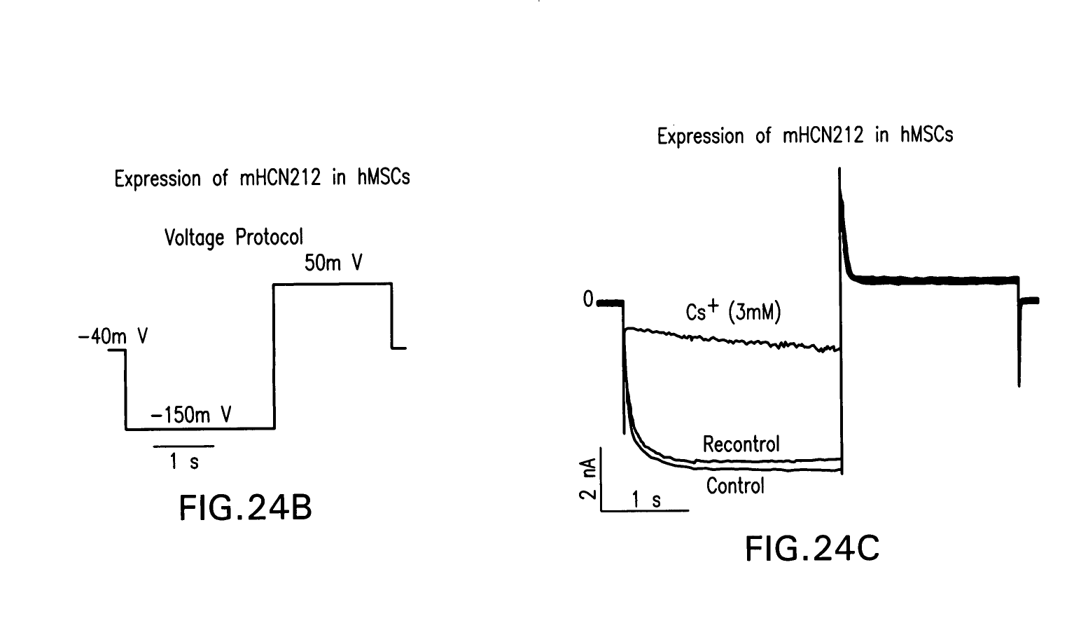

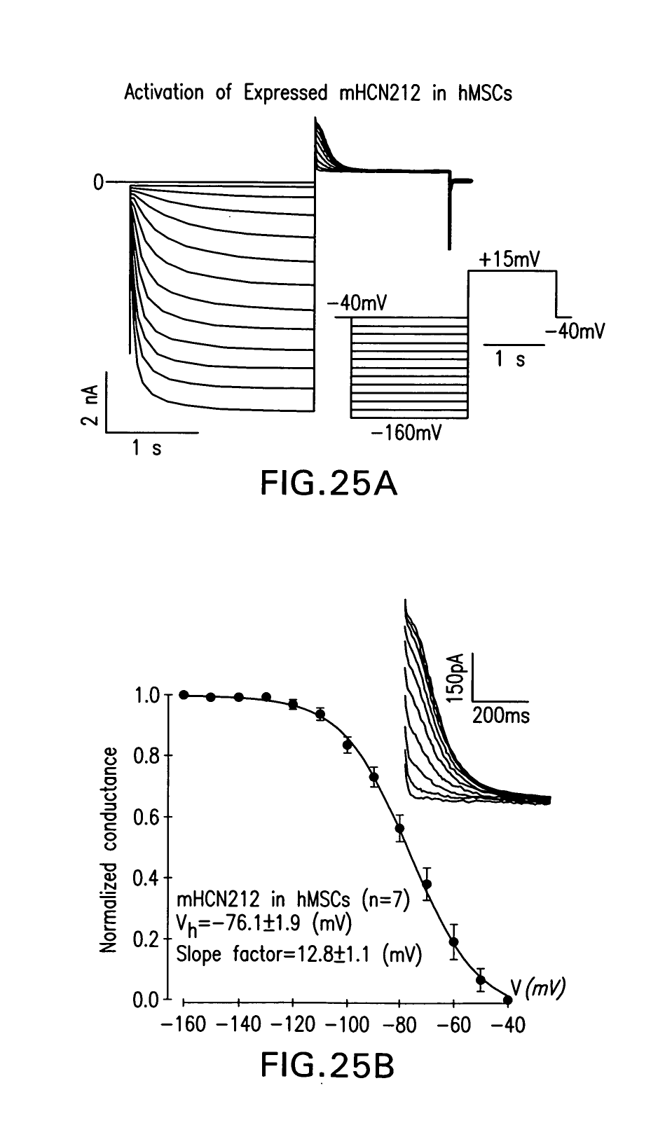

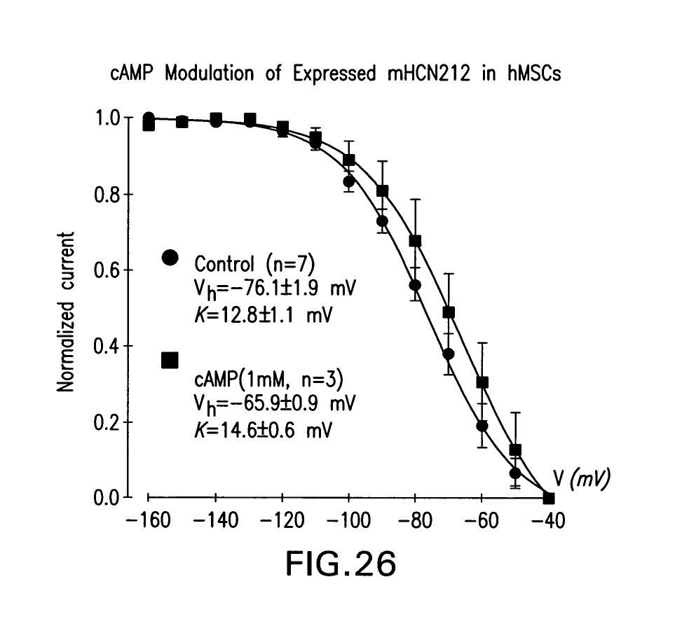

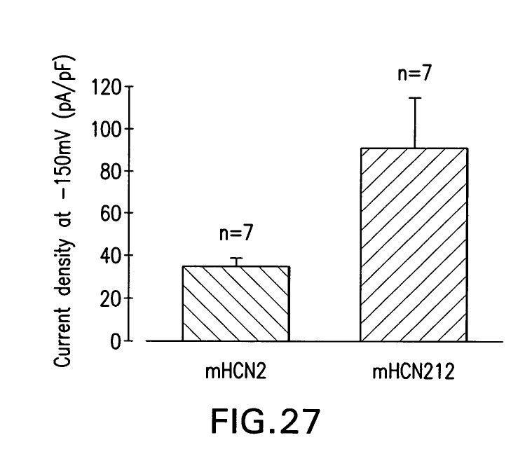

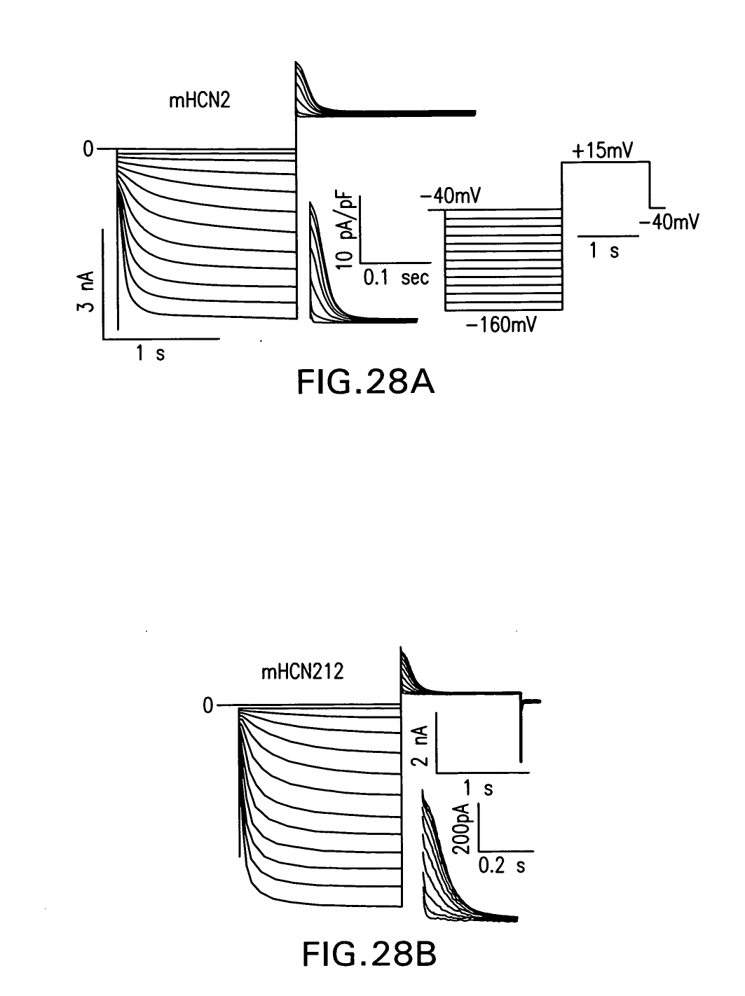

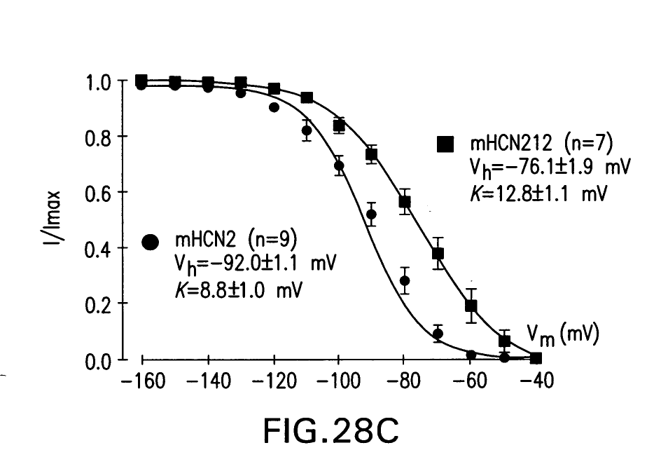

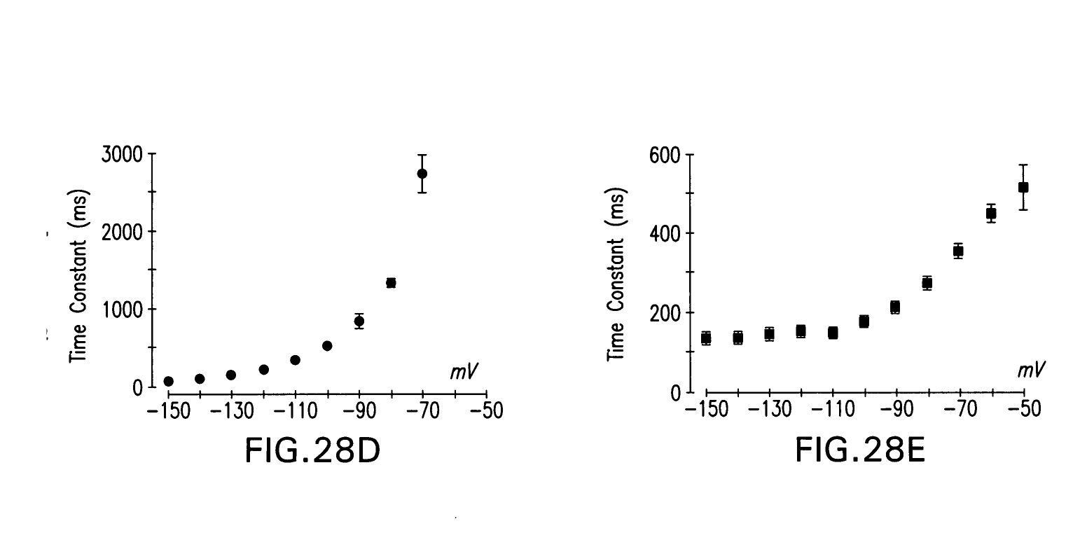

FIG. 21 demonstrates the response to epinephrine in terminal experiments. Panel A shows representative ECGs for all three groups prior to and during infusion of epinephrine, 1 μg/kg/min Control rates were 42, 44 and 52 bpm for the saline, mHCN2 and mE324A groups, respectively. With epinephrine, rates increased to 44, 60 and 81 bpm. Panel B summarizes the rate changes occurring at all doses of epinephrine. As can be seen, in the saline group all dogs showed less than a 50% increase in rate and/or ventricular premature depolarizations throughout the range of epinephrine concentrations administered. One-half of the mHCN2 group generated a 50% or more increase in heart rate, of which 33% required the highest dose of epinephrine to achieve this increase. The remainder had less than a 50% increase in heart rate or the occurrence of ventricular premature depolarizations. Finally, the mE324A group manifested greater than a 50% increase in heart rate at the lowest dose of epinephrine given. Hence there was far greater epinephrine sensitivity in the mE324A group than in either of the others.

Tandem Therapy as an Alternative to Either Electronic or Biological Pacemaking

The experimental data presented above demonstrate, inter alia, that biological pacemakers based on expression of mHCN2 and mE234A genes operate seamlessly in tandem with electronic pacemakers to prevent heart rate from falling below a selected minimum beating rate (FIG. 19); there is conservation of total number of electronic beats delivered (FIG. 20); and there is provision of a higher, more physiologic and catecholamine-responsive heart rate than is the case with an electronic pacemaker alone (FIG. 21). Although an adenoviral vector was used to introduce the pacemaker genes into canine hearts, data presented herein also indicate that hMSCs can provide an effective platform for delivery of ion channel currents into the heart. Factors favoring the use of hMSCs include their demonstrated ability to form gap junctions with a variety of cell types, including cardiomyocytes (FIGS. 10-13); their ability to generate in heart tissue pacemaker activity that appears to be stable, at least over a 6-week period (Plotnikov et al., 2005b); and evidence of no humoral or cellular rejection after six weeks (Plotnikov et al., 2005b), which if confirmed over the longer term, would abrogate any need for immunosuppression in hMSC-mediated therapy. Data were also provided indicating that HCN channel domains can be recombined to produce chimeric HCN channels that exhibit desirable gating characteristics for use in treating cardiac conditions.

The data provided herein confirm the feasibility of engineering a biologic pacemaker to meet the demands placed on modern day electronic pacemakers, specifically to provide a physiologic basal heart rate and a means to elevate heart rate during times of increased demand. mHCN2, mE324A and chimeric HCN channels provide biologic pacemakers with different characteristics; yet they demonstrate the principle that biologic pacemakers, like their electronic counterparts, can be tuned for basal heart rate and catecholamine responsiveness.

The strengths and weaknesses of electronic pacemakers have been previously considered (Rosen et al., 2004; Rosen, 2005; Cohen et al., 2005): clearly they are the state of the art as life-saving devices for treating a number of cardiac arrhythmias and are being used increasingly for cardiac failure. These advantages more than outweigh their disadvantages (see Background). Because electronic pacemakers represent a highly successful form of medical palliation, they will not easily be replaced, but the fact that they are not completely physiologic does make them a target for improvement and ultimately replacement. However, the therapy that replaces them should be more long-lasting, have less potential for inflicting damage, and be more physiologic. It is with this in mind that biological pacemakers are being developed. It has been suggested that biological pacemakers should have the potential to (1) create a lifelong, stable physiologic rhythm without need of replacement; (2) compete effectively with electronic pacemakers in satisfying the demand for a safe baseline rhythm, coupled with autonomic responsiveness to facilitate responsiveness to the demands of exercise and emotion; (3) be implanted at sites adjusted from one patient to another such that propagation through an optimal pathway of activation occurs and efficiency of contraction is optimized; (4) confer no risk of inflammation, neoplasia or rejection; (5) have no arrhythmogenic potential. In other words, they should represent not palliation, but cure (Rosen et al., 2004; Rosen, 2005).

There are two reasons to consider the use of tandem therapy as opposed to therapy based on biological or electronic pacemakers alone: one associated with clinical trials, and the other associated with more widespread clinical use. After the appropriate safety and efficacy preclinical testing is completed, a study of tandem pacemaking in patients in complete heart block and atrial fibrillation would be a reasonable starting point for a combined phase 1/phase 2 trial. Such a population has need of pacemaker therapy and is not a candidate for AV sequential electronic pacing. The state of the art therapy for such patients—a demand form of electronic ventricular pacing—would be indicated and a biological implant could be made as well. Moreover, the electronic component set at a sufficiently low rate would ensure a “safety net” in case the biological component failed. However, even if phase 1 and phase 2 trials provide evidence of safety and efficacy of the biological pacemaker there is a need to understand how long a biological pacemaker will last. And in the first generation of patients to receive them, this should likely be a lifelong question, during which there must be continued electronic backup.

With respect to broader clinical application of the tandem pacemaker concept there are several issues to consider. First, the system is redundant by design and would have two completely unrelated failure modes. Two independent implant sites and independent energy sources would provide a safety mechanism in the event of a loss of capture (e.g., due to myocardial infarction). Second, the electronic pacemaker would provide not only a baseline safety net, but an ongoing log of all heartbeats for review by clinicians, thus providing insight into a patient's evolving physiology and the performance of their tandem pacemaker system. Third, since the biologic pacemaker will be designed to perform the majority of cardiac pacing, the longevity of the electronic pacemaker could be dramatically improved. Alternatively longevity could be maintained while the electronic pacemaker could be further reduced in size. Finally, the biological component of a tandem system would provide true autonomic responsiveness, a goal that has eluded more than 40 years of electronic pacemaker research and development.

### EXAMPLE 6

### Biological-Electronic Biventricular Pacemakers

Up to about 50% of patients with advanced cardiac failure exhibit interventricular conduction delay (ventricular dyssynchrony) that may worsen LV systolic dysfunction through asynchronous ventricular contraction. Furthermore, prolonged QRS duration in these patients causes abnormal septal wall motion, reduced cardiac contractility, decreased diastolic filling time and extended mitral regurgitation. These abnormalities have been reported to be associated with increased morbidity and mortality. Biventricular pacing (cardiac resynchronization therapy; CRT) has been shown to be successful in coordinating the contraction of the ventricles and improving the hemodynamic status of the patient, thereby enhancing quality of life and reducing the risk of death of patients (see, e.g., Abraham and Hayes, 2003; Cleland et al., 2005).

To date, use of biventricular pacing to treat cardiac failure involves placing two leads in the ventricles in positions to optimize contraction. However, a problem that arises with this arrangement is that the leads cannot always be placed at sites where contraction can be optimized. The leads are inserted via the coronary veins (through catheterization of the coronary sinus) and distribution is limited to the sites of venous circulation. In contrast, a biological pacemaker can be implanted at any site in the ventricle via an endocardial approach, via the cardiac veins, or via thoracoscopy. Once it is located at an appropriate site in the ventricle to optimize contraction, the biological pacemaker may be used in a biventricular pacing mode in tandem with an electronic pacemaker. For an optimal biventricular pacing effect, the biological pacemaker is preferably implanted on the lateral free wall with a bias towards the apex rather than base. The electronic unit is programmed to sense and fire at a certain interval after the biological lead fires to provide biventricular function. In addition, the electronic pacemaker is also programmed to fire in an escape mode in the event the biological unit fires late.

In one embodiment, as an adjunct to the tandem system, a second electronic unit is placed in a coronary vein to function as a backup biventricular unit, and programmed to fire with the primary electronic unit in the event the biological component does not function. In another embodiment, nanoparticles are inserted in the stem cells, enabling the stem cells function as a capacitor to charge up and then fire in response to a signal emitted by the electronic unit. In yet another embodiment, nanoparticle-containing stem cells are used in tandem with an electronic pacemaker (that is, an electronic unit with a stem cell-nanoparticle unit working as a slave to it) to constitute a biventricular pacemaker. All of the above components, together with packaging material and a label providing instructions for use, may be combined in a kit for using the biological pacemaker in tandem with the electronic pacemaker in biventricular pacing mode to treat a subject afflicted with advanced cardiac failure and ventricular dyssynchrony.

In the development of a biological pacemaker, it seems likely that the use of an electronic pacemaker in tandem with a biologic cure will provide an essential bridge to the future of biologic therapeutics. While the bridge may lead to a future of pure biologic therapies, it may itself be an interesting destination providing greater benefit to patients and clinicians. The use of tandem biological and electronic pacemakers for biventricular pacing may be a particularly attractive destination.

### REFERENCES

U.S. Pat. No. 5,318,597, issued Jun. 7, 1994 to Hauck J A et al.
U.S. Pat. No. 5,376,106, issued Dec. 27, 1994 to Stahmann J E et al.
U.S. Pat. No. 5,983,138, issued Nov. 9, 1999 to Kramer A P.
U.S. Pat. No. 6,110,161, issued Aug. 29, 2000 to Mathiesen I et al.
U.S. Pat. No. 6,783,979, issued Aug. 31, 2004 to Rosen M R et al.
U.S. Pat. No. 6,849,611, issued Feb. 1, 2005 to Rosen M R et al.
U.S. Ser. No. 10/745,943, filed Dec. 24, 2003 by Rosen M R et al.
U.S. Ser. No. 10/342,506, filed Jan. 15, 2003 by Rosen M R et al., published Jul. 15, 2004 as U.S. Application Publication No. 2004/0137621 A1.
U.S. Provisional Application No. 60/532,363, filed Dec. 24, 2003 by Brink P R et al.
U.S. Provisional Application No. 60/701,312, filed Jul. 21, 2005 by Brink P R et al.
U.S. Provisional Application No. 60/704,210, filed Jul. 29, 2005 by Brink P R et al.
U.S. Provisional Application No. 60/715,934, filed Sep. 9, 2005 by Rosen M R et al.
PCT International Publication No. WO 2005/062857, published Jul. 14, 2005.
Abraham W T, Hayes D L (2003) Cardiac resynchronization therapy for heart failure. Circulation 108: 2596-2603.
Altomare C, Bucchi A, Camatini E, Baruscotti M, Viscomi C, Moroni A, DiFrancesco D (2001) Integrated allosteric model of voltage gating of HCN channels. J Gen Physiol 117: 519-532.
Amado R G, Chen I S (1999) Lentiviral Vectors—the promise of gene therapy within reach? Science 285: 674-676.
Biel M, Schneider A, Wahl C (2002) Cardiac HCN channels: Structure, function, and modulation. Trends Cardiovasc Med 12: 202-216.
Chen J, Mitcheson J S, Tristani-Firouzi M, Lin M, & Sanguinetti M C (2001a) The S4-S5 linker couples voltage sensing and activation of pacemaker channels. Proc Natl Acad Sci USA 98: 11277-11282.
Chen S, Wang J, Siegelbaum S A (2001b) Properties of hyperpolarization-activated pacemaker current defined by coassembly of HCN1 and HCN2 subunits and basal modulation by cyclic nucleotide. J Gen Physiol 117: 491-504.
Cho H C, Kashiwakura Y, Azene E, Marban E (2005) Conversion of non-excitable cells to self-contained biological pacemakers. Circulation (Abstract) 112: II-307.
Clapham D E (1998) Not so funny anymore: pacing channels are cloned. Neuron 21: 5-7.
Cleland J G, Daubert J C, Erdmann E, Freemantle N, Gras D, Kappenberger L, Tavazzi L, Cardiac Resynchronization-Heart Failure (CARE-HF) Study Investigators (2005) The effect of cardiac resynchronization on morbidity and mortality in heart failure. N Engl J Med 352:1539-1549.
Cohen I S, Brink, P R, Robinson R B, Rosen M R (2005) The why, what, how and when of biological pacemakers. Nat Clin Pract Cardiovasc Med 2: 374-375.
Decher N, Chen J, Sanguinetti M C (2004) Voltage-dependent gating of hyperpolarization-activated, cyclic nucleotide-gated pacemaker channels: molecular coupling between the S4-S5 and C-linkers. J Biol Chem 279: 13859-13865.
DiFrancesco D (1993) Pacemaker mechanisms in cardiac tissue. Annu Rev Physiol 55: 455-472.
Edelberg J M, Aird W C, Rosenberg R D (1998) Enhancement of murine cardiac chronotropy by the molecular transfer of the human β-adrenergic receptor cDNA. J Clin Invest 101: 337-343.
Edelberg J M, Huang D T, Josephson M E, Rosenberg R D (2001) Molecular enhancement of porcine cardiac chronotropy. Heart 86: 559-562.
Elfgang C, Eckert R, Lichtenberg-Frate H, Butterweck A, Traub O, Klein R A, Hulser D F, Willecke K. (1995) Specific permeability and selective formation of gap junction channels in connexin-transfected HeLa cells. J Cell Biol 129: 805-817.
Ferrer T, Rupp J, Piper D R, Tristani-Firouzi M (2006) The S4-S5 linker directly couples voltage sensor movement to the activation gate in the human ether-á-go-go-related gene (hERG) K⁺ channel. J Biol Chem 281:12858-12864.
Freudenberger R S, Wilson A C, Lawrence-Nelson J, Hare J M, Kostis J B; Myocardial Infarction Data Acquisition System Study Group (MIDAS 9) (2005) Permanent pacing is a risk factor for the development of heart failure. Am J Cardiol 95: 671-674.
Goulding E H, Ngai J, Kramer R H, Colicos S, Axel R, Siegelbaum S A, Chess A. (1992) Molecular cloning and single-channel properties of the cyclic nucleotide-gated channel from catfish olfactory neurons. Neuron 8: 45-58.
Harrison R L, Byrne B J, Tung 1 (1998) Electroporation-mediated gene transfer in cardiac tissue. FEBS Lett 435: 1-5.
Hayes D L (2000) Pacemaker timing cycles and electrocardiography, Chapter 6 of Cardiac Pacing and Defibrillation, pp. 201-223, Mayo Foundation.
He T C, Zhou S, da Costa L T, Yu J, Kinzler K W, Vogelstein B (1998) Simplified system for generating recombinant adenoviruses. Proc Natl Acad Sci USA 95: 2509-2514.
Ishii T M, Takano M, Xie L H, Noma A, Ohmori H (1999) Molecular characterization of the hyperpolarization-activated cation channel in rabbit heart sinoatrial node. J Biol Chem 274: 12835-12839.
Kehat, I, Khimovich L, Caspi O, Gepstein A, Shofti R, Arbel G, Huber I, Satin J, Iskovitz-Eldor J, Gepstein L (2004) Electromechanical integration of cardiomyocytes derived from human embryonic stem cells. Nature Biotech 22: 1282-1289.
Kuznetsov V Pak E, Robinson R B, Steinberg S F (1995) β-2-adrenergic receptor actions in neonatal and adult rat ventricular myocytes. Circ Res 76: 40-52.
Laing J G and Beyer E C (1995) The gap junction protein connexin43 is degraded via the ubiquitin proteasome pathway. J Biol Chem 270: 26399-26403.
Larsson H P (2002) The Search Is on for the Voltage Sensor-to-gate Coupling. J Gen Physiol 120: 475-481.
Liechty K W, MacKenzie T C, Shaaban A F, Radu A, Moseley A M, Deans R, Marshak D R, Flake A W (2000) Human mesenchymal stem cells engraft and demonstrate site-specific differentiation after in utero transplantation in sheep. Nat Med 6: 1282-1286.
Lu Y, Mahaut-Smith M P, Huang C L, Vandenberg J I (2003) Mutant MiRP1 subunits modulate HERG K+ channel gating: a mechanism for pro-arrhythmia in long QT syndrome type 6. J Physiol 551: 253-62.
Ludwig A, Zong X, Jeglitsch M, Hofmann F, Biel M (1998) A family of hyperpolarization-activated mammalian cation channels. Nature 393: 587-591.
Ludwig A, Zong X, Stieber J, Hullin R, Hofmann F, Biel M (1999) Two pacemaker channels from human heart with profoundly different activation kinetics. EMBO J. 18: 2323-2329.
Macri V, Accili EA (2004) Structural elements of instantaneous and slow gating in hyperpolarization-activated cyclic nucleotide-gated channels. J Biol Chem 279: 16832-16846.
Miake J, Marban E, Nuss H B (2003) Functional role of inward rectifier current in heart probed by Kir2.1 overexpression and dominant-negative-suppression. J Clin Invest 111: 1529-1536.
Miake J, Marban E, Nuss H B (2002) Gene therapy: biological pacemaker created by gene transfer. Gene therapy: biological pacemaker created by gene transfer. Nature 419: 132-133.
Moses H W, Moulton K P, Miller B D and Schneider J A (2000) Types of pacemakers and hemodynamics of pacing, Chapter 5 of A Practical Guide to Cardiac Pacing-Fifth Edition, pp. 78-84, Cippincott Williams & Wilkins, Philadelphia.
Mitcheson J S, Chen J, Sanguinetti M C (2000) Trapping of a methanesulfonanilide by closure of the HERG potassium channel activation gate. J Gen Physiol 115: 229-40.
Monteggia L M, Eisch A J, Tang M D, Kaczmarek L K, Nestler E J (2000) Cloning and localization of the hyperpolarization-activated cyclic nucleotide-gated channel family in rat brain. Brain Res Mol Brain Res 81: 129-139.
Ng P, Parks R J, Cummings D T, Evelegh C M, Graham F L (2000) An enhanced system for construction of adenoviral vectors by the two-plasmid rescue method. Hum Gene Ther 11: 693-699.
Orlic D, Kajstura J, Chimenti S, Jakoniuk I, Anderson S M, Li B, Pickel J, McKay R, Nadal-Ginard B, Bodine D M, Leri A, Anversa P (2001) Bone marrow cells regenerate infarcted myocardium. Nature 410: 701-705.
Pape H C (1996) Queer current and pacemaker: the hyperpolarization-activated cation current in neurons. Annu Rev Physiol 58: 299-327.
Perin E C, Geng Y J, Willerson J T (2003) Adult stem cell therapy in perspective. Circulation 107: 935-938.
Pfeifer A, Verma I M (2001) Gene therapy: promises and problems. Annu Rev Genomics Hum Genet @: 177-211.
Piper D R, Hinz W A, Tallurri C K, Sanguinetti M C, Tristani-Firouzi M (2005) Regional specificity of human ether-a-go-go-related gene channel activation and inactivation gating. J Biol Chem 2005 Feb. 25; 280(8):7206-17.
Pittenger M F, Mackay A M, Beck S C, Jaiswal R K, Douglas R, Mosca J D, Moorman M A, Simonetti D W, Craig S & Marshak D R (1999) Multilineage potential of adult human mesenchymal stem cells. Science 284: 143-147.
Plotnikov A N, Shlapakova I N, Kryukova Y, Bucchi A, Pan Z, Danilo P Jr, Brink P R, Cohen I S, Robinson R B, Rosen M R (2005a) Comparison of mHCN2 and mHCN2-E324A genes as biological pacemakers. Circulation 112: II-126 (Abstract).
Plotnikov A N, Shlapakova I N, Szabolcs M J, Danilo P, Jr., Lu Z, Potapova I, Lorell B H, Brink P R, Robinson R B, Cohen I S, Rosen M R (2005b) Adult human mesenchymal stem cells carrying HCN2 gene perform biological pacemaker function with no overt rejection for 6 weeks in canine heart. Circulation 112: II-221 (Abstract).
Plotnikov A N, Sosunov E A, Qu J, Shlapakova I N, Anyukhovsky E P, Liu L, Janse M J, Brink P R, Cohen I S, Robinson R B, Danilo P Jr, Rosen M R (2004) A biological pacemaker implanted in the canine left bundle branch provides ventricular escape rhythms having physiologically acceptable rates. Circulation 109: 506-512.
Potapova I, Plotnikov A, Lu Z, Danilo P Jr, Valiunas V, Qu J, Doronin S, Zuckerman J, Shlapakova I N, Gao J, Pan Z, Herron A J, Robinson R B, Brink P R, Rosen M R, Cohen I S (2004) Human mesenchynal stem cells as a gene delivery system to create cardiac pacemakers. Circ Res 94: 952-959.
Protas L, Robinson R B (1999) Neuropeptide Y contributes to innervation-dependent increase in I(Ca, L) via ventricular Y2 receptors. Am J Physiol 277: H940-946.
Robinson R B, Siegelbaum S A (2003) Hyperpolarization-activated cation currents: from molecules to physiological function. Annu Rev Physiol 65: 453-480.
Qu J, Barbuti A, Protas L, Santoro B, Cohen I S, Robinson R B (2001) HCN2 overexpression in newborn and adult ventricular myocytes: distinct effects on gating and excitability. Circ Res 89: E8-14.
Qu J, Kryukova Y, Potapova I A, Doronin S V, Larsen M, Krishnamurthy G, Cohen I S, Robinson R B (2004) MiRP1 modulates HCN2 channel expression and gating in cardiac myocytes. J Biol Chem 279: 43497-43502.
Qu J, Plotnikov A N, Danilo P Jr, Shlapakova I, Cohen I S, Robinson R B, Rosen M R (2003) Expression and function of a biological pacemaker in canine heart. Circulation 107: 1106-1109.
Rosen M R, Brink, P R, Cohen I S, Robinson R B (2004) Genes, stem cells and biological pacemakers. Cardiovasc Res 64: 12-23.
Rosen M (2005) Biological pacemaking: In our lifetime? Heart Rhythm 2: 418-428.
Sambrook J, Fritsch E F and Maniatis T (1989) Molecular Cloning: A Laboratory Manual, 2^{nd} ed., Cold Spring Harbor Laboratory Press, New York.
Santoro B, Chen S, Luthi A, Pavlidis P, Shumyatsky G P, Tibbs G R, Siegelbaum S A (2000) Molecular and functional heterogeneity of hyperpolarization-activated pacemaker channels in the mouse CNS. J Neurosci 20: 5264-5275.
Santoro B, Liu D T, Yao H, Bartsch D, Kandel E R, Siegelbaum S A, Tibbs G R (1998) Identification of a gene encoding a hyperpolarization-activated pacemaker channel of brain. Cell 93(5): 717-729.
Santoro B, Tibbs G R (1999) The HCN Gene Family: Molecular Basis of the Hyperpolarization-Activated Pacemaker Channels. Ann NY Acad Sci 868: 741-764.
Stieber J, Stockl G, Herrmann S, Hassfurth B, Hofmann F (2005) Functional expression of the human HCN3 channel. J Biol Chem 280: 34635-34643.
Strauer B E, Brehm M, Zeus T, Kostering M, Hernandez A, Sorg R V, Kogler G, Wernet P (2002) Repair of infarcted myocardium by autologous intracoronary mononuclear bone marrow cell transplantation in humans. Circulation 106; 1913-1918.
Trono D (2002) Lentiviral Vectors. Springer-Verlag: New York/Berlin/Heidelberg.
Tsang S Y, Lesso H, Li R A (2004) Dissecting the structural and functional roles of the S3-S4 linker of pacemaker (hyperpolarization-activated cyclic nucleotide-modulated) channels by systematic length alterations. J Biol Chem 279: 43752-43759.
Valiunas V, Beyer E C, Brink P R (2002) Cardiac gap junction channels show quantitative differences in selectivity. Circ Res 91: 104-111.
Valiunas V, Bukauskas F F, Weingart R (1997) Conductances and selective permeability of connexin43 gap junction channels examined in neonatal rat heart cells. Circ Res 80: 708-719.
Valiunas V, Doronin S, Valiuniene L, Potapova I, Zuckerman J, Walcott B, Robinson R B, Rosen M R, Brink P R, Cohen I S (2004) Human mesenchymal stem cells make cardiac connexins and form functional gap junctions. J Physiol 555: 617-26.
Valiunas V, Gemel J, Brink P R, Beyer E C (2001) Gap junction channels formed by coexpressed connexin40 and connexin43. Am J Physiol Heart Circ Physiol 281: H1675-H1689.
Valiunas V, Weingart R, Brink P R (2000) Formation of heterotypic gap junction channels by connexins 40 and 43. Circ Res 86: E42-E49.
van den Bos C, Mosca J D, Winkles J, Kerrigan L, Burgess W H & Marshak D R (1997) Human mesenchymal stem cells respond to fibroblast growth factors. Hum Cell 10: 45-50.
Vemana S, Pandey S, Larsson H P (2004) S4 movement in a mammalian HCN channel. J Gen Physiol 123: 21-32.
Wang H Z, Day N, Valcic M, Hsieh K, Serels S, Brink P R, Christ G J (2001a) Intercellular communication in cultured human vascular smooth muscle cells. Am J Physiol Cell Physiol 281: C75-C88.
Wang J, Chen S, Siegelbaum S A (2001b) Regulation of hyperpolarization-activated HCN channel gating and cAMP modulation due to interactions of COOH terminus and core transmembrane regions. J Gen Physiol 118: 237-250.
Wang Y, Bai Y, Price C, Boros P, Qin L, Bielinska A U, Kukowska-Latallo J F, Baker J R Jr, Bromberg J S (2001c) Combination of electroporation and DNA/dendrimer complexes enhances gene transfer into murine cardiac transplants. Am J Transplant 1: 334-338.
Xue T, Cho H C, Akar F G, Tsang S Y, Jones S P, Marban E, Tomaselli G F, Li R A (2005) Functional integration of electrically active cardiac derivatives from genetically engineered human embryonic stem cells with quiescent recipient ventricular cardiomyocytes. Circulation 111: 11-20.
Yu H, Gao J, Wang H, Wymore R, Steinberg S, McKinnon D, Rosen M R & Cohen I S (2000) Effects of the renin-angiotensin system on the current I(to) in epicardial and endocardial ventricular myocytes from the canine heart. Circ Res 86: 1062-1068.
Yu H, Lu Z, Pan Z, Cohen IS (2004) Tyrosine kinase inhibition differentially regulates heterologously expressed HCN channels. Pflugers Archiv. 447: 392-400.
Zhou Y Y, Wang S Q, Zhu W Z, Chruscinski A, Kobilka B K, Ziman B, Wang S, Lakatta E G, Cheng H & Xiao R P (2000) Culture and adenoviral infection of adult mouse cardiac myocytes: methods for cellular genetic physiology. Am J Physiol Heart Circ Physiol 279: H429-H436.

What is claimed is:

1. A tandem pacemaker system comprising (1) an electronic pacemaker, and (2) a biological pacemaker, wherein the biological pacemaker comprises an implantable cell that functionally expresses a hyperpolarization-activated, cyclic nucleotide-gated (HCN) ion channel, and wherein the expressed HCN channel generates an effective pacemaker current when the cell is implanted into a subjects heart.

2. The tandem pacemaker system of claim 1, wherein the cell is capable of gap junction mediated communication with cardiomyocytes.

3. The tandem pacemaker system of claim 2 wherein the cell is selected from the group consisting of a stem cell, a cardiomyocyte, a fibroblast or skeletal muscle cell engineered to express cardiac connexins, and an endothelial cell.

4. The tandem pacemaker system of claim 2 wherein the stem cell is an embryonic or adult stem cell.

5. The tandem pacemaker system of claim 4 wherein the stem cell is a human adult mesenchymal stem cell.

6. The tandem pacemaker system of claim 5 wherein the biological pacemaker comprises at least about 200,000 human adult mesenchymal stem cells.

7. The tandem pacemaker system of claim 5 wherein the biological pacemaker comprises at least about 700,000 human adult mesenchymal stem cells.

8. The tandem pacemaker system of claim 1 wherein the HCN channel is HCN1, HCN2, HCN3 or HCN4.

9. The tandem pacemaker system of claim 5 wherein the HCN channel is a human HCN1, HCN2, HCN3 or HCN4.

10. The tandem pacemaker system of claim 1 wherein the HCN channel has at least about 75% sequence identity with mHCN1 (SEQ ID NO: ______), mHCN2 (SEQ ID NO:______), mHCN3 (SEQ ID NO:______), or mHCN4 (SEQ ID NO:______).

11. The tandem pacemaker system of claim 9 wherein the HCN channel is a hHCN2 having SEQ ID NO: ______.

12. The tandem pacemaker system of claim 1 wherein the HCN channel is at least about 75% homologous to SEQ ID NO: ______.

13. The tandem pacemaker system of claim 1 wherein the cell further functionally expresses a MiRP1 beta subunit.

14. A tandem pacemaker system comprising (1) an electronic pacemaker, and (2) a biological pacemaker, wherein the biological pacemaker comprises an implantable cell that functionally expresses a mutant HCN ion channel, wherein the expressed mutant HCN channel generates an effective pacemaker current when implanted into a subject's heart.

15. The tandem pacemaker system of claim 14, wherein the cell functionally expresses a mutant HCN1, HCN2, HCN3, or HCN4.

16. The tandem pacemaker system of claim 15, wherein the cell functionally expresses a mutant human HCN1, HCN2, HCN3, or HCN4.

17. The tandem pacemaker system of claim 16, wherein the cell functionally expresses a mutant human HCN2.

18. The tandem pacemaker system of claim 14 wherein the mutant HCN channel provides an improved characteristic, as compared to a wild-type HCN channel, selected from the group consisting of faster kinetics, more positive activation, increased levels of expression, increased stability, enhanced cAMP responsiveness, and enhanced neurohumoral response.

19. The tandem pacemaker system of claim 14, wherein the mutant HCN channel contains a mutation in a region of the channel selected from the group consisting of the S4 voltage sensor, the S4-S5 linker, S5, S6 and S5-S6 linker, the C-linker, and the CNBD regions.

20. The tandem pacemaker system of claim 14 wherein the mutant HCN channel is derived from HCN2 and comprises E324A-HCN2, Y331A-HCN2, R339A-HCN2, or Y331A,E324A-HCN2.

21. The pacemaker system of claim 20, wherein the mutant HCN channel comprises E324A-HCN2.

22. The tandem pacemaker system of claim 14, wherein the cell is selected from the group consisting of a stem cell, a cardiomyocyte, a fibroblast or skeletal muscle cell engineered to express cardiac connexins, and an endothelial cell.

23. The tandem pacemaker system of claim 22 wherein the stem cell is an embryonic or adult stem cell and wherein said stem cell is substantially incapable of differentiation.

24. The tandem pacemaker system of claim 23 wherein the stern cell is a human adult mesenchymal stem cell.

25. The tandem pacemaker system of claim 24 wherein the biological pacemaker comprises at least about 200,000 human adult mesenchymal stem cells.

26. The tandem pacemaker system of claim 25 wherein the biological pacemaker comprises at least about 700,000 human adult mesenchymal stem cells.

27. A tandem pacemaker system comprising (1) an electronic pacemaker, and (2) a bypass bridge comprising a strip of gap junction-coupled cells having a first end and a second end, both ends capable of being attached to two selected sites in a heart, so as to allow the transmission of an electrical signal across the tract between the two sites in the heart.

28. The tandem pacemaker system of claim 27, wherein the first end of the bypass bridge is capable of being attached to the atrium and the second end capable of being attached to the ventricle, so as to allow transmission of an electrical signal from the atrium to travel across the tract to excite the ventricle.

29. The tandem pacemaker system of claim 27, wherein the cells are stem cells, cardiomyocytes, fibroblasts or skeletal muscle cells engineered to express cardiac connexins, or endothelial cells.

30. The tandem pacemaker system of claim 29 wherein the stem cell is an embryonic or adult stem cell.

31. The tandem pacemaker system of claim 30 wherein the stem cell is a human adult mesenchymal stem cell.

32. The tandem pacemaker system of claim 27, wherein the cells of the bypass bridge functionally express at least one protein selected from the group consisting of: a cardiac connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel.

33. The tandem pacemaker system of claim 27, wherein the cardiac connexin is selected from the group consisting of Cx43, Cx40, and Cx45.

34. The tandem pacemaker system of claim 27 further comprising a biological pacemaker comprising comprises an implantable cell that functionally expresses a
(a) an HCN ion channel, or
(b) a mutant HCN channel
wherein the expressed HCN, chimeric HCN or mutant HCN channel generates an effective pacemaker current when said cell is implanted into a subject's heart.

35. The tandem system of claim 34 wherein the implantable cell is a human adult mesenchymal stem cell.

36. The tandem system of claim 35 wherein the HCN channel is HCN2.

37. The tandem system of claim 35 wherein the biological pacemaker comprises at least about 200,000 human adult mesenchymal stem cells.

38. The tandem pacemaker system of claim 37 wherein the biological pacemaker comprises at least about 700,000 human adult mesenchymal stem cells.

39. A tandem pacemaker system comprising (1) an electronic pacemaker, and (2) a vector comprising a nucleic acid encoding an HCN channel or a mutant HCN channel, wherein said vector is administered to a cell in the heart of a subject and wherein said HCN channel or mutant HCN channel is expressed in the cells in the heart to generate an effective pacemaker current.

40. The tandem pacemaker system of claim 39 wherein the HCN channel is HCN2.

41. A method of treating a subject afflicted with a cardiac rhythm disorder, which method comprises administering the tandem pacemaker system of any of claims 1 or 14 to the subject, wherein the biological pacemaker of the system is provided to the subjects heart to generate an effective biological pacemaker current and further providing the electronic pacemaker to the subject's heart to work in tandem with the biological pacemaker to treat the cardiac rhythm disorder.

42. The method of claim 41 wherein the electronic pacemaker is provided before the biological pacemaker.

43. The method of claim 41 wherein the electronic pacemaker is provided simultaneously with the biological pacemaker.

44. The method of claim 41 wherein the electronic pacemaker is provided after the biological pacemaker.

45. The method of claim 41 wherein the biological pacemaker is provided to the Bachman's bundle, sinoatrial node, atrioventricular junctional region, His branch, left or right bundle branch, Purkinke fibers, right or left atrial muscle or ventricular muscle of the subject's heart.

46. The method of claim 41 wherein the biological pacemaker enhances beta-adrenergic responsiveness of the heart, decreases outward potassium current I_{K1}, and/or increases inward current I_{f}.

47. The method of claim 41 wherein the cardiac rhythm disorder is a sinus node dysfunction, sinus bradycardia, marginal pacemaker activity, sick sinus syndrome, tachyarrhythmia, sinus node reentry tachycardia, atrial tachycardia from an ectopic focus, atrial flutter, atrial fibrillation, bradyarrhythmia, or cardiac failure and wherein the biological pacemaker is administered to the left or right atrial muscle, sinoatrial node, or atrioventricular junctional region of the subject's heart.

48. The method of claim 41 wherein the electronic pacemaker is programmed to sense the subject's heart beating rate and to produce a pacemaker signal when the heart beating rate falls below a selected heart beating rate.

49. The method of claim 48 wherein the selected beating rate is a selected proportion of the beating rate experienced by the heart in a reference time interval.

50. The method of claim 49 wherein the reference time interval is an immediately preceding time period of selected duration.

51. The method of claim 50, wherein battery life of the electronic pacemaker is preserved.

52. A method of treating a cardiac rhythm disorder, wherein the disorder is a conduction block, complete atrioventricular block, incomplete atrioventricular block, bundle branch block, cardiac failure, or a bradyarrhythmia, the method comprising administering the pacemaker system of claim 27 to the subject's heart such that the bypass tract spans the region exhibiting defective conductance, wherein transmission by the bypass tract of an electronic pacemaker current induced by the electronic pacemaker is effective to treat the subject, and wherein the electronic pacemaker is provided either prior to, simultaneously with or after the bypass tract is provided.

53. A method of treating a cardiac rhythm disorder in a subject, wherein the disorder is a conduction block, complete atrioventricular block, incomplete atrioventricular block, bundle branch block, cardiac failure, or a bradyarrhythmia, the method comprising administering the pacemaker system of claim 1 or 14 to a region of the subject's heart to compensate for the conduction block.

54. The method of claim 52, wherein the electronic pacemaker is further programmed to sense the subject's heart beating rate and to produce a pacemaker signal when the heart beating rate falls below a selected heart beating rate.

55. A method of treating a subject afflicted with a sinus node dysfunction, sinus bradycardia, marginal pacemaker activity, sick sinus syndrome, cardiac failure, tachyarrhythmia, sinus node reentry tachycardia, atrial tachycardia from an ectopic focus, atrial flutter, atrial fibrillation, or a bradyarrhythmia and a conduction block disorder, which method comprises administering the tandem pacemaker system of claim 34, wherein the electronic pacemaker is provided either prior to, simultaneously with, or after the biological pacemaker is provided, and wherein the biological pacemaker is administered to the subject to generate an effective biological pacemaker current in the subject's heart, and wherein the bypass tract spans the region exhibiting defective conductance, wherein transmission by the bypass tract of an electronic pacemaker and/or biological pacemaker current is effective to treat the subject.

56. The method of claim 55, wherein the electronic pacemaker is further programmed to sense the subject's heart beating rate and to produce a pacemaker signal when the heart beating rate falls below a selected heart beating rate.

57. A method of monitoring cardiac signals in a subject the method comprising providing a tandem pacemaker system of claims 1 or 14 to a site in the subject's heart, wherein the electronic pacemaker is provided either prior to, simultaneously with, or after the biological pacemaker is provided, and monitoring the subject's heart rate with the electronic pacemaker.

58. The method of claim 57, wherein the electronic pacemaker is further programmed to sense the subject's heart beating rate and to produce a pacemaker signal when the heart beating rate falls below a selected heart beating rate.

59. A method of enhancing cardiac pacing function of an electronic pacemaker, the method comprising providing the tandem electronic pacemaker system of claims 1 or 14, and selectively stimulating the heart with the electronic pacemaker wherein the electronic pacemaker is programmed to sense the subject's heart beating rate and to produce a pacemaker signal when the heart beating rate falls below a selected heart beating rate.

60. A method of treating a subject afflicted with ventricular dyssynchrony comprising (a) selecting a site in a first ventricle of the subject's heart, (b) administering a biological pacemaker of claim 1 or 14 to the selected site so as to initiate pacemaker activity and stimulate contraction of the first ventricle, and (c) pacing a second ventricle of the heart with a first electronic pacemaker which is programmed to detect a signal from the biological pacemaker and to produce a pacemaker signal at a reference time interval after the biological pacemaker signal is detected, thereby providing biventricular pacemaker function to treat the subject.

61. The pacemaker system of claim 60 wherein the electronic pacemaker is further programmable to produce a pacemaker signal when it fails to detect a signal from the biological pacemaker after a time period of specified duration.

62. The pacemaker system of claim 60 further comprising a second electronic pacemaker to be administered to a coronary vein, wherein the second electronic pacemaker is programmable to detect a signal from the biological pacemaker and to produce a pacemaker signal in tandem with the first electronic pacemaker if said second electronic pacemaker fails to detect a signal from the biological pacemaker after a time period of specified duration, the first and second electronic pacemakers thereby providing biventricular function.

63. A tandem pacemaker system for treating a subject afflicted with ventricular dyssynchrony comprising (1) a biological pacemaker of claim 1 or 14 to be administered to a first ventricle of the subject's heart, and (2) an electronic pacemaker to be administered to a second ventricle of the subject's heart, wherein the electronic pacemaker is programmable to detect a signal from the biological pacemaker and to produce a electronic pacemaker signal at a reference time interval after the biological pacemaker signal is detected, so as to thereby provide biventricular pacemaker function, and wherein the electronic pacemaker is provided either prior or simultaneously with the biological pacemaker

64. The pacemaker system of claim 63 wherein the electronic pacemaker is further programmable to produce a pacemaker signal when it fails to detect a signal from the biological pacemaker after a time period of specified duration.

65. The pacemaker system of claim 63 further comprising a second electronic pacemaker to be administered to a coronary vein, wherein the second electronic pacemaker is programmable to detect a signal from the biological pacemaker and to produce a pacemaker signal in tandem with the first electronic pacemaker if said second electronic pacemaker fails to detect a signal from the biological pacemaker after a time period of specified duration, the first and second electronic pacemakers thereby providing biventricular function.
